(19) United States
(12) Patent Application Publication
(10) Pub. No.: US 2020/0063160 A1
(43) Pub. Date: Feb. 27, 2020

(54) METHOD FOR THE TREATMENT OF MUCOPOLYSACCHARIDOSIS TYPE II

(71) Applicant: Sangamo Therapeutics, Inc., Richmond, CA (US)

(72) Inventors: Dale Ando, Richmond, CA (US); Cheryl Wong Po Foo, Richmond, CA (US); Sagar A. Vaidya, Richmond, CA (US); Shelley Q. Wang, Richmond, CA (US)

(21) Appl. No.: 16/534,280

(22) Filed: Aug. 7, 2019

Related U.S. Application Data
(60) Provisional application No. 62/715,690, filed on Aug. 7, 2018.
(60) Provisional application No. 62/725,803, filed on Aug. 31, 2018.
(60) Provisional application No. 62/726,745, filed on Sep. 4, 2018.
(60) Provisional application No. 62/727,465, filed on Sep. 5, 2018.
(60) Provisional application No. 62/802,104, filed on Feb. 6, 2019.
(60) Provisional application No. 62/802,558, filed on Feb. 7, 2019.

(51) Int. Cl. C12N 15/86; C12N 9/22; A61P 43/00

(52) U.S. Cl. CPC C12N 15/86; C12N 9/22; A61K 45/06; C12N 2750/14143; A61P 43/00

(57) ABSTRACT

Described herein are methods and compositions for treating MPSII (Hunter) disease.

### CROSS-REFERENCE TO RELATED APPLICATIONS

The present application claims the benefit of U.S. Provisional Application No. 62/715,690, filed Aug. 7, 2018; U.S. Provisional Application No. 62/725,803, filed Aug. 31, 2018; U.S. Provisional Application No. 62/726,745, filed Sep. 4, 2018; U.S. Provisional Application No. 62/727,465, filed Sep. 5, 2018; U.S. Provisional Application No. 62/802,104, filed Feb. 6, 2019; and U.S. Provisional Application No. 62/802,558, filed Feb. 7, 2019, the disclosures of which are hereby incorporated by reference in their entireties.

### TECHNICAL FIELD

The present invention concerns methods for treating mucopolysaccharidosis type II (MPS II), also known as Hunter syndrome, and gene therapy.

### BACKGROUND

Lysosomal storage diseases (LSDs) are a group of rare metabolic monogenic diseases characterized by the lack of functional individual lysosomal proteins normally involved in the breakdown of cellular waste products, including lipids, mucopolysaccharides such as glycosoaminoglycans or GAGs. Mucopolysaccharidosis II (MPS II), also referred to as Hunter syndrome, is an X-linked, recessive, lysosomal storage disorder found predominantly in males (Burton & Giugliani (2012) Eur J Pediatr. (2012) April; 171(4):631-9). The disease is characterized by the accumulation of GAGs in the urine, plasma and tissues and causes multi-systemic, progressive disease (Muenzer (2014) Mol Gen Metabol 111:63-72). The life expectancy of untreated subjects with severe Hunter syndrome is into the mid teenage years with death due to neurologic deterioration and/or cardiorespiratory failure (Sato et al. (2013) Pediatr Cardiol. 34(8): 2077-2079).

The only currently approved therapy for MPS II is enzyme replacement therapy (ERT). Intravenous (IV) ERT with recombinant iduronate 2-sulfatase (IDS) protein (idursulfase; Elaprase®, Shire) has been US FDA approved since 2006 for administration once every week in a dose of 0.5 mg per kg of body weight and has been shown to improve walking capacity in MPS II subjects 5 years and older. Limitations to ERT include the need for life-long treatment, development of neutralizing antibodies, inability of the enzyme to cross the blood brain barrier, and the inconvenience of weekly intravenous infusions. Together, these limitations underscore the urgent need to develop a broader array of curative therapies for MPS II.

### SUMMARY

Disclosed herein are compositions and methods for treating and/or preventing Hunter syndrome (MPS II) in a subject. The present disclosure provides methods and compositions for genome editing and/or gene transfer. The disclosure provides methods of treating a subject with MPS II comprising administering one or more polynucleotides to the subject wherein the subject is treated. Methods of treatment provided herein include methods that reduce, delay, and/or eliminate additional treatment procedures and/or the onset, progression or severity of one or more symptoms associated with MPS II. In some embodiments, the methods of treatment provided herein include methods that reduce, stabilize or eliminate GAGs in the urine of a treated subject. In some embodiments, the methods reduce, stabilize or eliminate urinary GAG levels in a subject, including before, during and after additional treatment procedures. In some embodiments, the methods of treatment provided herein increase or stabilize the concentration of IDS in the plasma. In some embodiments, the methods of treatment provided herein result in a reduction, stabilization or elimination of urinary GAG levels while increasing or stabilizing the concentration of IDS in the plasma. In some embodiments, the methods of treatment provided herein result in a reduction, stabilization or elimination of urinary GAG levels wherein the concentration of IDS in the plasma is below the level of detection. In some embodiments, the total AAV dose includes two vectors comprising ZFN encoding sequences, and 1 vector comprising the IDS donor sequence in a fixed ratio of 1:1:8.

In some embodiments, additional treatment procedures that are reduced, delayed, and/or eliminated include enzyme replacement therapy (ERT) and/or bone marrow transplant and/or supportive surgical procedures for orthopedic, cardiac and/or upper airway obstruction. In some embodiments, the symptoms associated with MPS II whose onset, progression or severity are reduced, delayed or eliminated, include a decline in functional abilities, neurologic deterioration, joint stiffness, becoming wheelchair dependent, progression of disability, the requirement for forced air positive ventilation (requirement for a ventilator) and a shortened life span.

An objective and rationale for the compositions and methods provided herein is to use for example, in vivo genome editing to abrogate or decrease the need for enzyme replacement therapy. Methods of treatment provided herein employ an effective dose of engineered zinc finger nucleases (ZFNs) including to site-specifically integrate a corrective copy of the enzyme human iduronate-2-sulfatase (hIDS) transgene into the genome of a subject's own hepatocytes in vivo. In some embodiments, integration of the hIDS transgene is targeted to intron 1 of the albumin locus, resulting in stable, liver-specific expression and secretion of iduronate-2-sulfatase, measurable in the blood. In some embodiments, placement of the hIDS transgene under the control of the highly expressed endogenous albumin locus provides permanent, liver-specific expression of a subject with MPS II subject.

Disclosed herein are compositions and methods for treating a subject with MPS II comprising three polynucleotides: two polynucleotides encode partner halves (also referred to as a “paired ZFN” or “left and right ZFNs”) of a zinc finger nuclease and a third polynucleotide comprising a sequence encoding a functional iduronate-2-sulfatase (IDS) enzyme. In some embodiments, the zinc finger nuclease binds and cleaves the human albumin gene. Optionally, the nuclease-encoding polynucleotides further comprise sequences encoding small peptides (including but not limited to peptide tags and nuclear localization sequences), and/or comprise mutations in one or more of the DNA binding domain regions (e.g., the backbone of a zinc finger protein or TALE) and/or one or more mutations in a FokI nuclease cleavage domain or cleavage half domain. When these polynucleotide components are used individually or in any combination (e.g., peptide sequence such as FLAG, NLS, WPRE and/or poly A signal in any combination), the methods and compositions of the invention provide surprising and unexpected increases in expression of artificial nucleases with increased efficiency (e.g., 2, 3, 4, 5, 6, 10, 20 or more fold cleavage as compared to nucleases without the sequences/modifications described herein) and/or targeting specificity. In further embodiments, the polynucleotides encoding the zinc finger nuclease may comprise SB-47171 (SB-A6P-ZLEFT) or SB-47898 (SB-A6P-ZRIGHT) as disclosed herein. In further embodiments, the polynucleotides encoding the zinc finger nuclease may comprise SB-71557 (SB-A6P-ZL2) or SB-71728 (SB-A6P-ZR2). The composition may further comprise a polynucleotide comprising any donor nucleotide that encodes an iduronate-2-sulfatase (IDS) enzyme. In some embodiments, the donor nucleotide may comprise SB-IDS (SB-A6P-HNT) as disclosed herein. In some embodiments, the three polynucleotides are delivered to the subject with MPS II who is lacking an IDS gene such that a functional IDS protein is expressed in the subject. In some embodiments, the exogenous IDS gene is delivered to a cell in the subject together with the albumin-specific ZFN partner halves, such that the IDS gene is integrated (inserted) into the albumin gene. In further embodiments, the IDS gene expresses the IDS protein such that the subject with MPS II is treated. In some embodiments, the concentration of GAGs in the urine (e.g. urinary GAG levels) in the subject is reduced, stabilized or eliminated following administration of the composition and/or treatment according to the methods provided herein. In any of the methods described herein, IDS levels are increased to levels that treat the MPS II disease (e.g., symptoms) in the subject (e.g., normal range of IDS levels). The IDS levels (which provide the therapeutic benefits for example by reducing total GAG, reducing dermatan sulfate levels, reducing heparan sulfate levels, wherein the reductions are in the tissues and/or urine etc.) in the subject (including in any tissue or organ such as liver, plasma, urine, leukocytes, CNS, etc.) can be maintained for days, weeks, months, years or more following administration of the compositions described herein, including but not limited to 1 to 365 days (or any value therebetween such as 10, 30, 50, 90, 100, 150, 200, etc.).

In some embodiments, the composition comprises an effective dose of engineered zinc finger nucleases (ZFNs) to site-specifically integrate a corrective copy of a human enzyme iduronate-2-sulfatase (hIDS) transgene into the albumin locus of the subject's own hepatocytes in vivo. In some embodiments, the polynucleotides of the composition are carried on (delivered via) one or more AAV particles. In other embodiments, the AAV particles are AAV2/6 particles. The combination of the three AAV2/6 components, including the IDS donor AAV, Left ZFN AAV and Right ZFN AAV, is collectively a composition of the invention. Compositions and methods for treating a subject with MPS II are effective to provide hIDS which is active (functional) and able to degrade mucopolysaccharides glycosaminoglycans or GAGs in vivo in the subject such that the concentration of GAGs in the urine (e.g. urinary GAG level) is reduced, stabilized or eliminated following treatment and/or provide a measurable increase in the amount of active IDS in the plasma. Methods for insertion of a transgene sequence into the albumin locus are provided herein wherein the transgene encodes an hIDS protein (e.g., a functional full length or truncated IDS protein) that is expressed (e.g. is detectable in body fluids and tissues), the IDS protein is expressed and secreted or released from a hepatocyte comprising the transgene such that the expressed IDS protein is able to affect or be taken up by other cells that do not harbor the transgene (also referred to as a bystander effect or cross correction) and/or the IDS is active such that urine GAGs (e.g. total GAGs, DS-GAG and/or HS-GAG) is stabilized or decreased from baseline.

In some embodiments, provided herein are methods of treatment that reduce, delay, and/or eliminate additional treatment procedures as compared with a subject that has not been treated with the methods and compositions as disclosed herein, for example wherein an effective amount of hIDS transgene and zinc finger nucleases (ZFN) is administered to the subject, wherein the subject has a reduced, delayed, and/or eliminated need for additional treatment procedures after treatment. In some embodiments, the additional treatment procedures can include a bone marrow transplant, enzyme replacement therapy and/or surgical procedures for supportive treatment of cardiac, airway or orthopedic conditions associated with MPS II.

In some embodiments, the hIDS transgene (e.g. SEQ ID NO:15) useful in the compositions and methods described herein is delivered (e.g. to the hepatocyte) via AAV2/6 delivery, and an hIDS delivery vector further comprises homology arms (e.g. SEQ ID NO:13 and SEQ ID NO:16) flanking the hIDS transgene for example, with specificity for the regions flanking the ZFN cut site in the albumin locus. In some embodiments, the left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site. In some embodiments, the arms of homology are used to help facilitate targeted integration of the hIDS transgene at the albumin intron 1 locus via homology directed repair. In some embodiments, the size of the homology arms are chosen to avoid repetitive sequences and splicing elements in the albumin locus that can inhibit targeted integration or transgene expression. In some embodiments, the polyA sequences are derived from the bovine growth hormone gene. In some embodiments, the hIDS transgene donor further comprises a stop codon at the 3′ end, for example, to prevent further transcription of the endogenous albumin sequences into which the IDS transgene is inserted. In some embodiments, the rAAV2/6 donor vector containing the human IDS transgene (e.g. SB-IDS donor) is a promoterless construct that comprises a partial IDS cDNA comprising parts of exon 1 plus exons 2-9 (SEQ ID NO:15). In some embodiments, the splice acceptor site (e.g. SA, SEQ ID NO:14) derived from hF9 exon 2 is present, for example, to allow efficient splicing of the hIDS transcript into the mature mRNA from the albumin locus, and is effective with both types of the donor integration mechanisms (e.g. NHEJ or HDR). In some embodiments the donor comprises a sequence designated SB-IDS AAV (e.g. Table 3; SEQ ID NO:17).

In some embodiments, the ZFN useful in the compositions and methods disclosed herein (e.g., a ZFN in which the members of the ZFN pair (left and right) ZFNs are delivered on two separate vectors) include AAV vectors designated SB-47171 AAV and SB-47898 AAV as shown in Tables 1 and 2 and the sequences following these Tables, respectively. In further embodiments, the polynucleotides encoding the zinc finger nuclease may comprise SB-71557 (SB-A6P-ZL2) or SB-71728 (SB-A6P-ZR2). In some embodiments, the ZFNs in the albumin-specific pair are delivered (e.g. to the hepatocytes) via AAV2/6 delivery, for example, wherein one AAV comprises the left ZFN (e.g. SBS-47171; SEQ ID NO:9) and another comprises the right ZFN (e.g. SBS-47898; SEQ ID NO:12). In further embodiments, the polynucleotides encoding the zinc finger nuclease may comprise SB-71557 (SB-A6P-ZL2, SEQ ID NO:30) or SB-71728 (SB-A6P-ZR2, SEQ ID NO:31). In some embodiments, ZFN expression is under control of a liver-specific enhancer and promoter, for example, comprised of the human ApoE enhancer and human α1-anti-trypsin (hAAT) promoter (Miao C H et al. (2000) Mol. Ther. 1(6):522-532 (200)). In some embodiments, the liver specific promoter comprises one or more ApoE enhancer sequences (e.g., 1, 2, 3 and/or 4; see Okuyama et al. (1996) Hum Gen Ther 7(5):637-45). In some embodiments, the promoter is linked to an intron. In some embodiments, the intron is an HGG-IGG chimeric intron comprising the 5′ donor site from the first intron of the human β-globin gene and the branch and 3′ acceptor site from the intron of an immunoglobulin gene heavy chain variable region. In some embodiments, the ApoE/hAAT promoter (e.g. SEQ ID NO:2) is specifically and highly active in hepatocytes, the intended target tissue, but is inactive in non-liver cell and tissue types; this prevents ZFN expression and activity in non-target tissues. In some embodiments, the transthyretin minimal promoter is used (see U.S. Patent Publication No. 2017/0119906). In some embodiments, the composition comprises SB-47171 AAV (Table 1 and sequence following Table 1); SB-47898 (Table 2 and sequence following Table 2); and SB-IDS AAV (Table 3 and sequence following Table 3). In further embodiments, the composition comprises SB-71557 AAV (Table 4 and sequence following (e.g. SEQ ID NO:30)); SB-71728 AAV (Table 5 and sequence following (e.g. SEQ ID NO:31)); and SB-IDS AAV (Table 3 and sequence following Table 3 (e.g. SEQ ID NO:17)).

Optionally, the nuclease-encoding polynucleotides further comprise sequences encoding small peptides (including but not limited to peptide tags and nuclear localization sequences), and/or comprise mutations in one or more of the DNA binding domain regions (e.g., the backbone of a zinc finger protein or TALE) and/or one or more mutations in a FokI nuclease cleavage domain or cleavage half domain. When these polynucleotide components are used individually or in any combination (e.g., peptide sequence such as FLAG, NLS, WPRE and/or poly A signal in any combination), the methods and compositions of the invention provide surprising and unexpected increases in expression of artificial nucleases with increased efficiency (e.g., 2, 3, 4, 5, 6, 10, 20 or more fold cleavage as compared to nucleases without the sequences/modifications described herein) and/or targeting specificity. In some embodiments, the nuclease is encoded by an mRNA and the mRNA optionally comprises elements for increasing transcriptional and translational efficiency. In some embodiments, the elements comprise untranslated sequences such as natural or artificial 5′ and/or 3′ UTR sequences. In some aspects, a 5′ UTR sequence is included in an expression cassette, while in others, a 3′ UTR sequence is used. In some embodiments, an mRNA encoding an artificial nuclease comprises both a 5′ UTR and a 3′ UTR. In one embodiment, the 5′ UTR is a Xenopus β-globin UTR (see Krieg and Melton (1994) Nuc Acid Res 12(18):7057). In some embodiments, the DNA sequence encoding the Xenopus β-globin UTR is 5′ TGCTTGTTCTTTTTGCAGAAGCTCAGAATAAACGCTCAACTTTGGCAGAT (SEQ ID NO:18). In some embodiments, the mRNA encoding the nuclease comprises a 3′ WPRE sequence (see U.S. Patent Publication No. 2016/0326548). In some embodiments, the WPRE element is a mutated in the ‘X’ region to prevent expression of Protein X (see U.S. Pat. No. 7,419,829). In some embodiments, the mutated WPRE element comprises mutations described in Zanta-Boussif et al. (2009) Gene Ther 16(5):605-619. In some embodiments, the WPRE is a WPRE3 variant (Choi et al. (2014) Mol Brain 7:17). In some embodiments, the 3′ UTR comprises a poly A signal sequence. The poly A signal may be 3′ or 5′ to the WPRE sequence when these elements are used in combination. In some embodiments, the poly A signal sequence is the bovine Growth Hormone signal sequence (see Woychik et al. (1984) Proc Natl Acad Sci 81(13):3944-8).

The methods and compositions of the invention can also include mutations to one or more amino acids within the DNA binding domain outside the residues that recognize the nucleotides of the target sequence (e.g., one or more mutations to the ‘ZFP backbone’ (outside the DNA recognition helix region)) that can interact non-specifically with phosphates on the DNA backbone. Thus, in some embodiments, the methods and compositions disclosed herein includes mutations of cationic amino acid residues in the ZFP backbone that are not required for nucleotide target specificity. In some embodiments, these mutations in the ZFP backbone comprise mutating a cationic amino acid residue to a neutral or anionic amino acid residue. In some embodiments, these mutations in the ZFP backbone comprise mutating a polar amino acid residue to a neutral or non-polar amino acid residue. In some embodiments, mutations at made at position (−5), (−9) and/or position (−14) relative to the DNA binding helix. In some embodiments, a zinc finger may comprise one or more mutations at (−5), (−9) and/or (−14). In some embodiments, one or more zinc fingers in a multi-finger zinc finger protein may comprise mutations in (−5), (−9) and/or (−14). In some embodiments, the amino acids at (−5), (−9) and/or (−14) (e.g. an arginine (R) or lysine (K)) are mutated to an alanine (A), leucine (L), Ser (S), Asp (N), Glu (E), Tyr (Y) and/or glutamine (Q). See, e.g., U.S. Publication No. 20180087072.

In some aspects, the methods and compositions of the invention include the use of sequences encoding exogenous peptide sequences fused to eukaryotic transgene sequences. In some embodiments, exogenous peptides are fused to protein sequences post-translationally, and in other embodiments, the sequences encoding the exogenous peptides are linked in frame (3′ and/or 5′) to sequences encoding the artificial nuclease (e.g., a fusion protein). The exogenous peptides may encode sequences useful for purification or labeling, e.g. affinity purification or immunohistochemistry. Examples of such peptides are polyhistidine tags (“His tag”, Hochuli et al. (1988) Bio/Technol 6(11):1321-5) or cationic peptide tags such as Flag tags (Hopp et al. (1988) Bio/Technol 6(10):1204-10). One or more (1, 2, 3, 4, 5 or more) of these peptide tag sequences can be used in any combinations. In some embodiments, the sequence encoding an exogenous Flag peptide comprising the sequence N-term DYKDDDK (SEQ ID NO:19) is fused in frame at the C-terminus or N-terminus of a sequence encoding an artificial nuclease. In preferred embodiments, a sequence encoding 3 FLAG sequences (3× FLAG peptide) is used (see U.S. Pat. No. 6,379,903), wherein the amino acid sequence is N-term DYKDHDG-DYKDHDI-DYKDDDDK (SEQ ID NO:20). Inclusion of one or more of such peptides sequences (e.g., 3× FLAG) can increase nuclease (cleavage) activity by 2, 3, 4, 5, 6, 7, 8, 9, 10, 11 or more fold) as compared to nucleases without the peptide sequences.

In some aspects, the mRNA encoding an artificial nuclease comprises a nuclear localization peptide sequence (NLS). In some embodiments, the NLS comprises the sequence PKKKRKV (SEQ ID NO:21) from the SV40 virus large T gene (see Kalderon et al. (1984) Nature 311(5981):33-8) while in others, the NLS comprises the sequence PAAKRVKLD (SEQ ID NO:22) from the c-myc protein (see Dang and Lee (1988) Mol Cell Biol 8(10):4048-54). In some embodiments, the NLS comprises the sequence EGAPPAKRAR (SEQ ID NO:23) from the hepatitis delta virus (see Alves et al. (2008) Virology 370:12-21) or VSRKRPRP (SEQ ID NO:24) from the polyoma T protein (Richardson et al. (1986) Cell 44(1):77-85). In other embodiments, the NLS comprises the sequence KRPAATKKAGQAKKKKLD (SEQ ID NO:25), derived from the nucleoplasmin carboxy tail (see Dingwall (1988) J Cell Biol 107:841-849 and Robbins et al. (1991) Cell 64(3):615-23), while in some embodiments, the NLS comprises the sequence NQS SNFGPMKGGNFGGRSSGPYGGGGQYFAKPRNQGGY (SEQ ID NO:26) first described by Siomi and Dreyfuss (Siomi and Dreyfus (1995) J Cell Biol 129(3):551-560). In further embodiments, the NLS comprises the sequence PKTRRRPRRSQRKRPPT (SEQ ID NO:27) from the Rex protein in HTLV-1 (Siomi et al. (1988) Cell 55(2):197-209). Inclusion of one or more of NLS sequences as described herein can increase nuclease (cleavage) activity by 2, 3, 4, 5, 6, 7, 8, 9, 10, 11 or more fold) as compared to nucleases without the peptide sequences.

In some embodiments, the need for additional therapeutic procedures, such as bone marrow transplant, ERT therapy and/or supportive surgical procedures, in the subject is delayed, reduced or eliminated in the subject after treatment. In some embodiments, the delayed, reduced or eliminated need for additional therapeutic procedures is measured by a change in IDS activity and/or level in the plasma and/or leukocytes and/or tissues (including for example, blood, liver tissue and CSF). In some embodiments, the activity and/or level of IDS in the plasma and/or in leukocytes and/or blood, liver tissue and CSF is increased, stays the same or is below the level of detection following treatment. Methods to detect IDS in the plasma and/or subject leukocytes are known in the art. See for example Chuang et al. (2018) Orphanet J Rare Dis. 13:84. In some embodiments, the delayed, reduced or eliminated need for additional therapeutic procedures in the subject is measured, for example, by a change in total GAG, DS GAG (GAG comprising dermatan sulfate), and HS GAG (GAG comprising heparan sulfate) levels (for example, expressed as a ratio to creatinine) measured in the treated subject's urine. In some embodiments, the delayed, reduced or eliminated need for additional therapeutic procedures is measured, for example, by a change from baseline in forced vital capacity measured by a pulmonary function test. In some embodiments, the delayed, reduced or eliminated need for additional therapeutic procedures is measured by a change from base line, for example, in distance walked as measured by a 6-minute walk test of the subject. In some embodiments, the delayed, reduced or eliminated need for additional therapeutic procedures in the subject is measured, for example, by a change from baseline in joint range of motion (JROM). In some embodiments, the delayed, reduced or eliminated need for additional therapeutic procedures in the subject is measured, for example, by a change from baseline in spleen and/or liver volume, for example as measured by MRI (before and after treatment). In some embodiments, the delayed, reduced or eliminated need for additional therapeutic procedures is measured, for example, by a change from baseline in neurocognitive abilities as measured, for example, by WASI-II (Wechsler Abbreviated Scale of Intelligence, Second Edition (Shapiro et al. (2015) Mol Genet Metab 116(1-2):61-68), WPPSI-IV (Wechsler Preschool and Primary Scale of Intelligence), or BSID-III (Bayley Scales of Infant Development), and by VABS-II (Vineland Adaptive Behavior Scales). In some embodiments, the delayed, reduced or eliminated need for additional therapeutic procedures is measured, for example, by a change from baseline in total GAG, DS GAG, and HS GAG levels measured, for example, in blood, liver tissue and CSF.

In some embodiments, the subject has received ERT at baseline or has received ERT in the past, while in other embodiments, the subject has not received ERT.

In some embodiments, the methods and compositions disclosed herein comprise dosing of a composition of the invention, for example, via a peripheral vein catheter. In some embodiments, the composition is added to a normal saline (NS) or phosphate buffered saline (PBS) diluent, wherein the diluent may further comprise, for example, human serum albumin. In some embodiments, the subject receives a total AAV dose, for example, of 5e12 vg/kg comprising 5e11 vg/kg of each ZFN AAV2/6 comprising either a left ZFN or a right ZFN (e.g., SB-47171 AAV and SB-47898 AAV or SB-71557 AAV and SB-71728 AAV), and 4e12 vg/kg of a hIDS donor AAV (e.g., SB-IDS AAV). In some embodiments, the subject receives a total AAV dose, for example, of 1e13 vg/kg comprising 1e12 vg/kg of each ZFN AAV2/6 comprising, for example, either a left ZFN or a right ZFN, and 8e12 vg/kg of the hIDS donor AAV. In further embodiments, the subject receives a total AAV dose, for example, of 5e13 vg/kg comprising 5e12 vg/kg of each ZFN AAV comprising, for example, either a left ZFN or a right ZFN, and 4e13 of the hIDS donor AAV. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. The components may be administered separately, or, preferably a composition comprising all components (paired ZFNs on the same or different vectors and IDS donor), for example, a composition which comprises SB-47171 AAV (Table 1), SB-47898 AAV (Table 2) and SB-IDS AAV (Table 3). In some embodiments, the composition comprises SB-71557 AAV (Table 4, SEQ ID NO:30), SB-71728 AAV (Table 5, SEQ ID NO:31) and SB-IDS AAV (Table 3, SEQ ID NO: 17).

In some embodiments, the subject has delayed, reduced or eliminated need, for example, for additional therapeutic procedures after receiving a total dose of 5e12 vg/kg of the composition, of 1e13 vg/kg of the composition, of 5e13 vg/kg of the composition, of 1e14 vg/kg of the composition, of 5e14 vg/kg of the composition and/or 1e15 vg/kg of the composition. In some embodiments, the subject has delayed, reduced or eliminated need, for example, for additional therapeutic procedures after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In another aspect, disclosed herein is a method of reducing, delaying or eliminating the symptoms in a subject with MPS II as compared with a subject that has not been treated with the methods and compositions of the invention, the method comprising, for example, administering to the subject an effective amount of hIDS transgene and zinc finger nucleases (ZFN) wherein the subject has reduced, delayed or eliminated symptoms after treatment. In some embodiments, organomegaly, hyperactivity, aggressiveness, neurologic deterioration, joint stiffness, skeletal deformities, heart valve thickening, hearing loss, hernias, and/or upper respiratory infections are reduced, delayed or eliminated by the compositions and methods disclosed herein. In some embodiments, the hIDS transgene (e.g. SEQ ID NO:15) is delivered (e.g. to the hepatocyte) via AAV2/6 delivery, and the hIDS delivery vector (e.g.as shown in SB-IDS AAV, Table 3, e.g. SEQ ID NO:17), which further comprises homology arms (e.g. SEQ ID NO:13 and SEQ ID NO:16) flanking the hIDS transgene with specificity for the regions flanking the ZFN cut site, for example, in the albumin locus. The left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site of the ZFNs useful in the methods and compositions disclosed herein. In some embodiments, the arms of homology are used to help facilitate targeted integration, for example, of the hIDS transgene at the albumin intron 1 locus (e.g. via homology directed repair). In some embodiments, the size of the homology arms are chosen to avoid repetitive sequences and splicing elements, for example, in the albumin locus that can inhibit targeted integration or transgene expression. In some embodiments, the polyA sequences are derived from the bovine growth hormone gene. In some embodiments, the hIDS transgene donor further comprises a stop codon at the 3′ end, for example, to prevent further transcription of the albumin sequences into which the IDS transgene is inserted. In some embodiments, the rAAV2/6 donor vector containing the human IDS transgene (e.g. SB-IDS donor) is a promoterless construct that comprises a partial IDS cDNA comprising parts of exon 1 plus exons 2-9 (SEQ ID NO:15). The splice acceptor site (SA, SEQ ID NO:14) derived from hF9 exon 2 is present to allow efficient splicing of the hIDS transcript into the mature mRNA from the albumin locus, and is effective with both types of the donor integration mechanisms (NHEJ or HDR).

In some embodiments, the ZFNs in the albumin-specific pair are similarly delivered to the hepatocytes via AAV2/6 delivery wherein one AAV comprises the left ZFN (SBS-47171; SEQ ID NO:9) and another comprises the right ZFN (SBS-47898; SEQ ID NO:12). In some embodiments, the ZFNs in the albumin-specific pair are delivered to the hepatocytes via AAV2/6 delivery wherein one AAV comprises the left ZFN (SBS-71557; SEQ ID NO:30) and another comprises the right ZFN (SBS-71728; SEQ ID NO:31). In some embodiments, the ZFN comprises two separate polynucleotides (carried on AAV vectors): SB-47171 AAV (e.g. Table 1, SEQ ID NO:9) and SB-47898 (e.g. Table 2, SEQ ID NO:12). In some embodiments, the ZFN comprises two separate polynucleotides (carried on AAV vectors): SB-71557 AAV (e.g. Table 4, SEQ ID NO:30) and SB-71728 (e.g. Table 5, SEQ ID NO:31). In some embodiments, ZFN expression is under control of a liver-specific enhancer and promoter, comprised of, for example, the human ApoE enhancer and human α1-anti-trypsin (hAAT) promoter (Miao C H et al. (2000) Mol. Ther. 1(6):522-532 (200)). In some embodiments, the ApoE/hAAT promoter (e.g. SEQ ID NO:2) is specifically and highly active in hepatocytes, the intended target tissue in some embodiments, but is inactive in non-liver cell and tissue types; this prevents ZFN expression and activity in non-target tissues. In some embodiments, ZFN expression is under the minimal transthyretin promoter. In some embodiments, the expression cassette comprising a ZFN comprises one or more FLAG tags, a nuclear localization sequence (NLS), a WPRE sequence, an alternate poly A sequence, a 5′ UTR or a 3′ UTR as described above. In some embodiments, the composition comprises SB-47171 AAV (e.g. Table 1, SEQ ID NO:9); SB-47898 (e.g. Table 2, SEQ ID NO:12); and SB-IDS AAV (e.g. Table 3, SEQ ID NO:17). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, reduced, delayed or eliminated MPS II symptoms in the subject after treatment is measured by a change in IDS activity or level in the plasma by comparing activity or level before and after treatment. In some embodiments, the activity and/or level of IDS in the plasma increases, stays the same, or is below the level of detection. In some embodiments, reduced, delayed or eliminated MPS II symptoms in the subject after treatment is measured, for example, by a change in total GAG, DS GAG (e.g. GAG comprising dermatan sulfate), and HS GAG (e.g. GAG comprising heparan sulfate) levels (expressed as a ratio to creatinine) measured in the treated subject's urine. In some embodiments, reduced, delayed or eliminated MPS II symptoms in the subject after treatment is measured, for example, by a change from baseline or a stabilization in forced vital capacity measured by a pulmonary function test. In some embodiments, reduced, delayed or eliminated MPS II symptoms in the subject after treatment is measured, for example, by a change or stabilization from base line in distance walked as measured by the subject performing a 6-minute walk test before and after treatment to determine the change from base line due to treatment. In some embodiments, reduced, delayed or eliminated MPS II symptoms in the subject after treatment is measured, for example, by a change from baseline or a stabilization in joint range of motion (JROM). In some embodiments, reduced, delayed or eliminated MPS II symptoms in the subject after treatment is measured, for example, by a change from baseline or a stabilization in spleen and/or liver volume as measured by MRI. In some embodiments, reduced, delayed or eliminated MPS II symptoms in the subject after treatment is measured, for example, by a change from baseline or stabilization in neurocognitive abilities as measured by WASI-II (Wechsler Abbreviated Scale of Intelligence, Second Edition (Shapiro et al., ibid)), WPPSI-IV (Wechsler Preschool and Primary Scale of Intelligence), or BSID-III (Bayley Scales of Infant Development), and by VABS-II (Vineland Adaptive Behavior Scales). In some embodiments, reduced, delayed or eliminated MPS II symptoms in the subject after treatment is measured, for example, by a change from baseline or stabilization in total GAG, DS GAG, and HS GAG levels measured in liver tissue and CSF.

In some embodiments, the subject has received ERT at baseline or has received ERT in the past, while in other embodiments, the subject has not received ERT.

In some embodiments, the methods and compositions disclosed herein comprise dosing of the composition, for example, via a peripheral vein catheter. In some embodiments, the composition is added to a normal saline (NS) or phosphate buffered saline (PBS) diluent, wherein the diluent may further comprise, for example, human serum albumin. In some embodiments, the subject receives a total AAV dose, for example, of 5e12 vg/kg comprising 5e11 vg/kg of each ZFN AAV2/6 comprising either a left ZFN or a right ZFN, and 4e12 vg/kg of the hIDS donor AAV. In other embodiments, the subject receives a total AAV dose, for example, of 1e13 vg/kg comprising 1e12 vg/kg of each ZFN AAV2/6 comprising either a left ZFN or a right ZFN, and 8e12 vg/kg of the hIDS donor AAV. In further embodiments, the subject receives a total AAV dose, for example, of 5e13 vg/kg comprising 5e12 vg/kg of each ZFN AAV comprising either a left ZFN or a right ZFN, and 4e13 of the hIDS donor AAV. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. The method and compositions disclosed herein may be administered separately, or, preferably a composition comprising all components (e.g. paired ZFNs on the same or different vectors and IDS donor), for example a composition which comprises SB-47171 AAV (e.g. Table 1), SB-47898 AAV (e.g. Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the reduced, delayed or eliminated MPS II symptoms exhibited in the subject after use of the methods and compositions disclosed herein with a composition of the invention is seen when the subject receives a total dose, for example, of 5e12 vg/kg, of 1e13 vg/kg, of 5e13 vg/kg, of 1e14 vg/kg, of 5e14 vg/kg and/or 1e15 vg/kg. In some embodiments, the subject has reduced, delayed, or eliminated MPS II symptoms after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In some embodiments, methods and compositions as disclosed herein of delaying the need for ERT initiation in a subject with MPS II as compared with a subject that has not been treated with the methods and compositions of the invention as disclosed herein, the methods comprising administering to the subject, for example, an effective amount of hIDS transgene and zinc finger nucleases (ZFN) useful in the invention, wherein the need for ERT in the subject is delayed after treatment. The hIDS transgene (e.g. SEQ ID NO:15) is delivered (e.g. to the hepatocyte) via AAV2/6 delivery, and the hIDS delivery vector further comprises, for example, homology arms (e.g. SEQ ID NO:13 and SEQ ID NO:16) flanking the hIDS transgene with specificity, for example, for the regions flanking the ZFN cut site in the albumin locus. The left arm of homology (LA) contains about 280 nucleotides (e.g. SEQ ID NO:13), for example, of identical sequence upstream of the albumin intron 1 cleavage site, and the right arm of homology (RA) contains, for example, about 100 nucleotides (e.g. SEQ ID NO:16) of identical sequence downstream of the cleavage site. In some embodiments, the arms of homology are used, for example, to help facilitate targeted integration of the hIDS transgene at the albumin intron 1 locus via homology directed repair. In some embodiments, the size of the homology arms are chosen, for example, to avoid repetitive sequences and splicing elements in the albumin locus that can inhibit targeted integration or transgene expression. The polyA sequences are derived from the bovine growth hormone gene. In some embodiments, the hIDS transgene donor further comprises, for example, a stop codon at the 3′ end to prevent further transcription of the albumin sequences into which the IDS transgene is inserted. In some embodiments, the rAAV2/6 donor vector containing the human IDS transgene (e.g. SB-IDS donor) is a promoterless construct that comprises a partial IDS cDNA comprising parts of exon 1 plus exons 2-9 (e.g. SEQ ID NO:15). In some embodiments, the splice acceptor site (e.g. SA, SEQ ID NO:14), for example, derived from hF9 exon 2, is present to allow efficient splicing of the hIDS transcript into the mature mRNA from the albumin locus, and is effective with both types of the donor integration mechanisms (e.g. NHEJ or HDR).

In some embodiments the ZFNs useful for the compositions and methods disclosed herein are similarly delivered (e.g. to the hepatocytes) via AAV2/6 delivery. In some embodiments, the ZFN is albumin-specific, for example, and the halves (left and right components) of the albumin-specific ZFNs are carried by separate AAV vectors. In some embodiments, one AAV comprises the left ZFN (e.g. SBS-47171; SEQ ID NO:9) and another comprises the right ZFN (e.g. SBS-47898; SEQ ID NO:12). In some embodiments, one AAV comprises the left ZFN (e.g. SB-71557, Table 4, SEQ ID NO:30); and another comprises the right ZFN (e.g. SB-71728 Table 5, SEQ ID NO:31). In some embodiments, expression of the ZFNs useful in the methods and compositions disclosed herein is under control of a liver-specific enhancer and promoter, for example, comprised of the human ApoE enhancer and human α1-anti-trypsin (hAAT) promoter (Miao C H et al. (2000) Mol. Ther. 1(6): 522-532 (200)). In some embodiments, the ApoE/hAAT promoter (e.g. SEQ ID NO:2) is specifically and highly active (e.g. in hepatocytes and/or the intended target tissue), but is inactive in non-liver cell and tissue types; this prevents ZFN expression and activity in non-target tissues. In some embodiments, the AAV vectors comprise SB-47171 AAV (e.g. Table 1) and SB-47898 (e.g. Table 2). In some embodiments, the composition administered comprises SB-47171 AAV (e.g. Table 1, SEQ ID NO:9); SB-47898 (e.g. Table 2, SEQ ID NO:12); and SB-IDS AAV (e.g. Table 3, SEQ ID NO:17). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the delayed or reduced need for ERT is measured, for example, in the subject after treatment. In some embodiments, the delayed need for ERT is measured, for example, by a change in IDS activity or level in the plasma. In some embodiments, the activity and/or level of IDS in the plasma, CSF, liver and/or leukocytes is increased, stays the same, or is below the level of detection. In some embodiments, the delayed need for ERT is measured, for example, by a change or stabilization in total GAG, DS GAG (e.g. GAG comprising dermatan sulfate), and HS GAG (e.g. GAG comprising heparan sulfate) levels (for example, expressed as a ratio to creatinine) measured in the treated subject's urine (e.g. urine GAG level). In some embodiments, the delayed need for ERT is measured, for example, by a change from baseline or stabilization in forced vital capacity measured by a pulmonary function test. In some embodiments, the delayed need for ERT is measured, for example, by a change from base line or stabilization in distance walked as measured by a 6-minute walk test. In some embodiments, the delayed need for ERT is measured, for example, by a change from baseline or stabilization in joint range of motion (JROM). In some embodiments, the delayed need for ERT is measured, for example, by a change from baseline or stabilization in spleen and/or liver volume as measured by MM. In some embodiments, the delayed need for ERT is measured, for example, by a change from baseline or stabilization in neurocognitive abilities as measured by WASI-II (Wechsler Abbreviated Scale of Intelligence, Second Edition (Shapiro et al., ibid)), WPPSI-IV (Wechsler Preschool and Primary Scale of Intelligence), or BSID-III (Bayley Scales of Infant Development), and by VABS-II (Vineland Adaptive Behavior Scales). In some embodiments, the delayed need for ERT is measured, for example, by a change from baseline or stabilization in total GAG, DS GAG, and HS GAG levels in liver tissue and CSF.

In some embodiments, the subject has received ERT at baseline or has received ERT in the past, while in other embodiments, the subject has not received ERT.

In some embodiments, the treatment comprises dosing of the composition, for example, via a peripheral vein catheter. In some embodiments, the composition is added to a normal saline (NS) or phosphate buffered saline (PBS) diluent. In some embodiments, the subject receives a total AAV dose, for example, of 5e12 vg/kg comprising 5e11 vg/kg of each ZFN AAV2/6 comprising either a left ZFN or a right ZFN (e.g., SB-47171 AAV and SB-47898 AAV), and 4e12 vg/kg of the hIDS donor AAV (e.g., SB-IDS AAV). In some embodiments, the subject receives a total AAV dose, for example, of 1e13 vg/kg comprising 1e12 vg/kg of each ZFN AAV2/6 comprising either a left ZFN or a right ZFN (e.g., SB-47171 AAV or SB-71557 AAV and SB-47898 AAV or SB 71728 AAV), and 8e12 vg/kg of the hIDS donor AAV (e.g., SB-IDS AAV). In some embodiments, the subject receives a total AAV dose, for example, of 5e13 vg/kg comprising 5e12 vg/kg of each ZFN AAV comprising either a left ZFN or a right ZFN (e.g., SB-47171 AAV or SB-71557 and SB-47898 AAV or SB 71728 AAV), and 4e13 of the hIDS donor AAV (e.g., SB-IDS AAV). In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. In some embodiments, the components may be administered separately, or, preferably as a composition comprising all components (for example, paired ZFNs on the same or different vectors and IDS donor), for example a composition which comprises SB-47171 AAV or SB-71557 (e.g. Table 1 or Table 4), SB-47898 AAV or SB-71728 (e.g. Table 2 or Table 5) and SB-IDS AAV (e.g. Table 3).

In some embodiments, the delayed need for ERT is measured for the subject, for example, after treatment with a composition with a total dose of 5e12 vg/kg, of 1e13 vg/kg, of 5e13 vg/kg, of 1e14 vg/kg, of 5e14 vg/kg and/or 1e15 vg/kg. In some embodiments, the delayed need for ERT is measured after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In another aspect, disclosed herein is a method for removing (withdrawing) ERT in a subject with MPS II, the method comprising, for example, (a) administering to a subject receiving ERT an effective amount of an hIDS transgene and zinc finger nucleases (ZFN) as described herein; and (b) withdrawing ERT from the subject after step (a). The ERT may be withdrawn at any time after administration, including, hours (0-48), days (1-7 days), weeks (1-4 weeks), months (1-12) or years (1-10 years) after administration of the transgene and ZFN(s). In certain embodiments, ERT is withdrawn completely while in other embodiments, ERT may be withdrawn for any period of time, including for example, a longer period of time as compared to a subject that has not been administered the transgene and ZFN(s). In some embodiments, the methods may further comprise assessing the ability to withdraw ERT in a subject by, for example, measuring one or more symptoms associated with MPS II, for example by assessing changes in organomegaly, hyperactivity, aggressiveness, neurologic deterioration, joint stiffness, skeletal deformities, heart valve thickening, hearing loss, hernias, and/or upper respiratory infections in the subject following administration of the transgene and ZFN(s), wherein if the measurements demonstrate that one or more of these (MPS II) symptoms are reduced, delayed or eliminated by the compositions and methods disclosed herein such that ERT is no longer needed. In some embodiments, the method comprises a hIDS transgene (e.g. SEQ ID NO:15) that is delivered (e.g. to the hepatocyte) via AAV2/6 delivery, and the hIDS delivery vector (e.g. as shown in SB-IDS AAV, Table 3, e.g. SEQ ID NO:17), which further comprises homology arms (e.g. SEQ ID NO:13 and SEQ ID NO:16) flanking the hIDS transgene with specificity for the regions flanking the ZFN cut site, for example, in the albumin locus. The left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site of the ZFNs useful in the methods and compositions disclosed herein. In some embodiments, the arms of homology are used to help facilitate targeted integration, for example, of the hIDS transgene at the albumin intron 1 locus (e.g. via homology directed repair). In some embodiments, the size of the homology arms are chosen to avoid repetitive sequences and splicing elements, for example, in the albumin locus that can inhibit targeted integration or transgene expression. In some embodiments, the polyA sequences are derived from the bovine growth hormone gene. In some embodiments, the hIDS transgene donor further comprises a stop codon at the 3′ end, for example, to prevent further transcription of the albumin sequences into which the IDS transgene is inserted. In some embodiments, the rAAV2/6 donor vector containing the human IDS transgene (e.g. SB-IDS donor) is a promoterless construct that comprises a partial IDS cDNA comprising parts of exon 1 plus exons 2-9 (SEQ ID NO:15). The splice acceptor site (SA, SEQ ID NO:14) derived from hF9 exon 2 is present to allow efficient splicing of the hIDS transcript into the mature mRNA from the albumin locus, and is effective with both types of the donor integration mechanisms (NHEJ or HDR).

In some embodiments, the ZFNs in the albumin-specific pair are similarly delivered to the hepatocytes via AAV2/6 delivery wherein one AAV comprises the left ZFN (SBS-47171 or SB-71557; SEQ ID NO:9 or SEQ ID NO:30, respectively) and another comprises the right ZFN (SBS-47898 or SB-71728; SEQ ID NO:12 or SEQ ID NO:31, respectively). In some embodiments, the ZFN comprises two separate polynucleotides (carried on AAV vectors): SB-47171 AAV or SB-71557 (e.g. Table 1 or Table 4, SEQ ID NO:9 or SEQ ID NO:30, respectively) and SB-47898 or SB-71728 (e.g. Table 2 or Table 5, SEQ ID NO:12 or SEQ ID NO:31, respectively). In some embodiments, ZFN expression is under control of a liver-specific enhancer and promoter, comprised of, for example, the human ApoE enhancer and human α1-anti-trypsin (hAAT) promoter (Miao C H et al. (2000) Mol. Ther. 1(6):522-532 (200)). In some embodiments, the ApoE/hAAT promoter (e.g. SEQ ID NO:2) is specifically and highly active in hepatocytes, the intended target tissue in some embodiments, but is inactive in non-liver cell and tissue types; this prevents ZFN expression and activity in non-target tissues. In some embodiments, the composition comprises SB-47171 AAV (e.g. Table 1, SEQ ID NO:9); SB-47898 (e.g. Table 2, SEQ ID NO:12); and SB-IDS AAV (e.g. Table 3, SEQ ID NO:17). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, withdrawal of ERT in a subject with MPS II after treatment with the methods and compositions disclosed herein is assessed by one or more of the following before and after treatment: measuring a change or stabilization in IDS activity or level in the plasma, CSF, liver or in leukocytes as between before and after treatment, in which increased IDS activity after treatment is indicative that ERT can be delayed or withdrawn ; measuring a change or stabilization in total GAG, DS GAG (e.g. GAG comprising dermatan sulfate), and/or HS GAG (e.g. GAG comprising heparan sulfate) levels (expressed as a ratio to creatinine) in the treated subject's urine as between before and after treatment, wherein a reduction or stabilization in levels of total GAG, DS GAG and/or HS GAG after treatment is indicative that ERT can be withdrawn or delayed; measuring a change from baseline or stabilization in forced vital capacity measured by a pulmonary function test as between before and after treatment, wherein an increase or stabilization in the forced vital capacity after treatment is indicative that ERT can be withdrawn or delayed; measuring a change from base line or stabilization in distance walked as measured by the subject performing a 6 minute walk test before and after treatment to determine the change from base line due to treatment, wherein an increase or stabilization in the distance walked by the subject after treatment is indicative that ERT can be withdrawn or delayed; measuring a change from baseline or stabilization in joint range of motion (JROM) as between before and after treatment, wherein an increase or stabilization in the range of motion after treatment is indicative that ERT can be withdrawn; measuring a change from baseline or stabilization in spleen and/or liver volume as measured by MRI as between before and after treatment, wherein a decrease or stabilization in the spleen and/or liver volume after treatment is indicative that ERT can be withdrawn or delayed; measuring a change from baseline or stabilization (before treatment) in neurocognitive abilities as measured by WASI-II (Wechsler Abbreviated Scale of Intelligence, Second Edition (Shapiro et al., ibid)), WPPSI-IV (Wechsler Preschool and Primary Scale of Intelligence), or BSID-III (Bayley Scales of Infant Development), and by VABS-II (Vineland Adaptive Behavior Scales), wherein improvement or stabilization in neurocognitive abilities as between baseline (before) and after treatment are indicative that ERT can be withdrawn or delayed; and/or measuring a change from baseline in total GAG, DS GAG, and/or HS GAG levels measured in liver tissue and CSF before and after treatment, wherein a reduction or stabilization in total GAG, DS GAG and/or HS GAG levels after treatment are indicative that ERT can be withdrawn or delayed. ERT may thus be withdrawn or delayed in which a positive change or a stabilization is seen in one or more of these assessments after treatment (as compared to before treatment (baseline)). In some embodiments, the subject has received ERT at baseline or has received ERT in the past.

In some embodiments, the methods and compositions disclosed herein comprise dosing of the composition, for example, via a peripheral vein catheter. In some embodiments, the composition is added to a normal saline (NS) or phosphate buffered saline (PBS) diluent, wherein the diluent may further comprise, for example, human serum albumin. In some embodiments, the subject receives a total AAV dose, for example, of 5e12 vg/kg comprising 5e11 vg/kg of each ZFN AAV2/6 comprising either a left ZFN or a right ZFN, and 4e12 vg/kg of the hIDS donor AAV. In other embodiments, the subject receives a total AAV dose, for example, of 1e13 vg/kg comprising 1e12 vg/kg of each ZFN AAV2/6 comprising either a left ZFN or a right ZFN, and 8e12 vg/kg of the hIDS donor AAV. In further embodiments, the subject receives a total AAV dose, for example, of 5e13 vg/kg comprising 5e12 vg/kg of each ZFN AAV comprising either a left ZFN or a right ZFN, and 4e13 of the hIDS donor AAV. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. The method and compositions disclosed herein may be administered separately, or, preferably a composition comprising all components (e.g. paired ZFNs on the same or different vectors and IDS donor), for example a composition which comprises SB-47171 AAV (e.g. Table 1), SB-47898 AAV (e.g. Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the ability to withdraw ERT in the subject after use of the methods and compositions disclosed herein with a composition of the invention is seen when the subject receives a total dose, for example, of 5e12 vg/kg, of 1e13 vg/kg, of 5e13 vg/kg, of 1e14 vg/kg, of 5e14 vg/kg and/or 1e15 vg/kg. In some embodiments, the ability to withdraw ERT in the subject after use of the methods and compositions disclosed herein is seen after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In some embodiment, provided herein is a method of delaying, reducing or preventing the need for a bone marrow transplant in a subject with MPS II as compared with a subject that has not been treated with the methods and compositions of the invention as disclosed herein, the method comprising administering to the subject an effective amount of hIDS transgene and zinc finger nucleases (ZFN) wherein the subject has a delayed, reduced or prevented need, for example, for a bone marrow transplant after treatment with the methods and compositions disclosed herein. In some embodiments, the hIDS transgene (e.g. SEQ ID NO:15) is delivered (e.g. to the hepatocyte) via AAV2/6 delivery, and the hIDS delivery vector further comprises homology arms (e.g. SEQ ID NO:13 and SEQ ID NO:16) flanking the hIDS transgene with specificity for the regions flanking the ZFN cut site in the albumin locus. The left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site. In some embodiments, the arms of homology are used, for example, to help facilitate targeted integration of the hIDS transgene at the albumin intron 1 locus (e.g. via homology directed repair). In some embodiments, the size of the homology arms are chosen, for example, to avoid repetitive sequences and splicing elements in the albumin locus that can inhibit targeted integration or transgene expression. In some embodiment, the polyA sequences are derived from the bovine growth hormone gene. In some embodiments, the hIDS transgene donor further comprises, for example, a stop codon at the 3′ end to prevent further transcription of the albumin sequences into which the IDS transgene is inserted. In some embodiments, the rAAV2/6 donor vector containing the human IDS transgene (e.g. SB-IDS donor) is a promoterless construct that comprises a partial IDS cDNA comprising parts of exon 1 plus exons 2-9 (e.g. SEQ ID NO:15). In some embodiments, the splice acceptor site (e.g. SA, SEQ ID NO:14) is derived, for example, from hF9 exon 2 to allow efficient splicing of the hIDS transcript, for example, into the mature mRNA from the albumin locus, and is effective with both types of the donor integration mechanisms (e.g. NHEJ or HDR). In some embodiments, the donor is the donor designated SB-IDS AAV (e.g. Table 3, SEQ ID NO:17).

In some embodiments, the ZFNs useful in the methods and compositions disclosed herein delivered to the subject are an albumin-specific pair (e.g. delivered to the hepatocytes) via AAV2/6 delivery wherein one AAV comprises the left ZFN (e.g. SBS-47171 or SBS-71557; SEQ ID NO:9 or SEQ ID NO:30, respectively) and another comprises the right ZFN (e.g. SBS-47898 or SBS-71728; SEQ ID NO:12 or SEQ ID NO:31, respectively). In some embodiments, ZFN expression is under control, for example, of a liver-specific enhancer and promoter, comprised of the human ApoE enhancer and human α1-anti-trypsin (hAAT) promoter (Miao C H et al. (2000) Mol. Ther. 1(6): 522-532 (200)). In some embodiments, ZFN expression is under the minimal transthyretin promoter. In some embodiments, the expression cassette comprising a ZFN comprises one or more FLAG tags, a nuclear localization sequence (NLS), a WPRE sequence, an alternate poly A sequence, a 5′ UTR or a 3′ UTR as described above. In some embodiments, the ApoE/hAAT promoter (e.g. SEQ ID NO:2) is specifically and highly active (e.g. in hepatocytes, the intended target tissue), but is inactive in non-liver cell and tissue types; this prevents ZFN expression and activity in non-target tissues. In some embodiments, the ZFN pair useful in the methods and compositions disclosed herein is delivered using two separate AAV vectors, namely SB-47171 AAV or SB-71557 (e.g. Table 1, SEQ ID NO:9 or Table 4, SEQ ID NO:30, respectively) and SB-47898 AAV or SB-71728 (e.g. Table 2, SEQ ID NO:12 or Table 5, SEQ ID NO:31, respectively). In some embodiments, any of the methods and compositions described herein may use a three component AAV system (2 AAVs for each component of a paired ZFN and 1 AAV carrying the donor), for example a composition which comprises SB-47171 AAV (e.g. Table 1), SB-47898 AAV (e.g. Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the delayed, reduced or prevented need for a bone marrow transplant is measured in the subject after treatment using the methods and compositions disclosed herein. In some embodiments, the delayed, reduced or prevented need for a bone marrow transplant is measured by a change in IDS activity or level in the plasma. In some embodiments, the activity and/or level of IDS in the plasma, CSF, liver or in the leukocytes increases, stays the same, or is below the level of detection. In some embodiments, the delayed, reduced or prevented need for a bone marrow transplant is measured by a change in IDS activity or level in the subject's leukocytes. In some embodiments, the delayed, reduced or prevented need for a bone marrow transplant is measured by a change or stabilization in total GAG, DS GAG (e.g. GAG comprising dermatan sulfate), and HS GAG (e.g. GAG comprising heparan sulfate) levels (for example, expressed as a ratio to creatinine) measured in the treated subject's urine (e.g. urine GAG levels). In some embodiments, the delayed, reduced or prevented need for a bone marrow transplant is measured, for example, by a change from baseline or stabilization in forced vital capacity measured by a pulmonary function test. In some embodiments, the delayed or reduced need for a bone marrow transplant is measured, for example, by a change from base line or stabilization in distance walked as measured by a 6-minute walk test. In some embodiments, the delayed or reduced need for a bone marrow transplant is measured, for example, by a change from baseline or stabilization in joint range of motion (JROM). In some embodiments, the need for a bone marrow transplant is decreased by a change from baseline or stabilization in spleen and/or liver volume as measured, for example, by MRI. In some embodiments, the reduced, delayed or prevented need for a bone marrow transplant is measured, for example, by a change from baseline or stabilization in neurocognitive abilities as measured by WASI-II (Wechsler Abbreviated Scale of Intelligence, Second Edition (Shapiro et al., ibid)), WPPSI-IV (Wechsler Preschool and Primary Scale of Intelligence), or BSID-III (Bayley Scales of Infant Development), and by VABS-II (Vineland Adaptive Behavior Scales). In some embodiments, the reduced or delayed need for ERT is measured, for example, by a change from baseline or stabilization in total GAG, DS GAG, and HS GAG levels measured in liver tissue and CSF.

In some embodiments, the subject has received ERT at baseline, while in other embodiments, the subject has not received ERT.

In some embodiments, the methods and compositions disclosed herein comprises dosing of a composition (e.g. via a peripheral vein catheter). In some embodiments, the composition is added to a normal saline (NS) or phosphate buffered saline (PBS) diluent, wherein the diluent further comprises, for example, human serum albumin. In some embodiments, the subject receives a total AAV dose, for example, of 5e12 vg/kg comprising 5e11 vg/kg of each ZFN AAV2/6 comprising either a left ZFN or a right ZFN, and 4e12 vg/kg of the hIDS donor AAV as disclosed herein. In some embodiments, the subject receives a total AAV dose, for example, of 1e13 vg/kg comprising 1e12 vg/kg of each ZFN AAV2/6 comprising either a left ZFN or a right ZFN, and 8e12 vg/kg of the hIDS donor AAV as disclosed herein. In some embodiments, the subject receives a total AAV dose, for example, of 5e13 vg/kg comprising 5e12 vg/kg of each ZFN AAV comprising either a left ZFN or a right ZFN, and 4e13 of the hIDS donor AAV as disclosed herein. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. In some embodiments, the components may be administered separately, or, preferably a composition comprising all components (paired ZFNs on the same or different vectors and IDS donor), for example a composition which comprises SB-47171 AAV (e.g. Table 1), SB-47898 AAV (e.g. Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the reduced, delayed or prevented need for a bone marrow transplant is measured for the subject after treatment with the methods and compositions disclosed herein, comprising a total dose of, for example, 5e12 vg/kg, of 1e13 vg/kg, of 5e13 vg/kg, of 1e14 vg/kg, of 5 vg/kg and/or 1e15 vg/kg. In some embodiments, reduced, delayed or prevented need for a bond marrow transplant is measured for the subject after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In some embodiments, provided herein is a method of reducing, stabilizing or eliminating urine GAGs (e.g. urine GAG levels) by treatment with the methods and compositions disclosed herein as compared with a subject that has not been treated, the method comprising, for example, administering to the subject an effective amount of nuclease(s) and donor(s) as described herein (e.g., a three-component composition comprising an hIDS transgene and zinc finger nucleases (ZFN)), wherein the subject has reduced, stabilized or eliminated urine GAGs (e.g. urine GAG levels) after treatment. In some embodiments, the activity or level of IDS in the plasma is below the level of detection. In some embodiments, the hIDS transgene (e.g. SEQ ID NO:15) is delivered (e.g. to the hepatocyte) via AAV2/6 delivery, and the hIDS delivery vector further comprises, for example, homology arms (e.g. SEQ ID NO:13 and SEQ ID NO:16) flanking the hIDS transgene with specificity for the regions flanking the ZFN cut site in the albumin locus. In some embodiments, the left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site. In some embodiments, the arms of homology are used to help facilitate targeted integration, for example, of the hIDS transgene at the albumin intron 1 locus via homology directed repair. In some embodiments, the size of the homology arms are chosen, for example, to avoid repetitive sequences and splicing elements in the albumin locus that can inhibit targeted integration or transgene expression. In some embodiments, the polyA sequences are derived from the bovine growth hormone gene. In some embodiments, the hIDS transgene donor further comprises, for example, a stop codon at the 3′ end to prevent further transcription of the albumin sequences into which the IDS transgene is inserted. In some embodiments, the rAAV2/6 donor vector comprising the human IDS transgene (e.g. SB-IDS donor) is a promoterless construct that comprises a partial IDS cDNA comprising parts of exon 1 plus exons 2-9 (e.g. SEQ ID NO:15). In some embodiments, the splice acceptor site (e.g. SA, SEQ ID NO:14), for example, derived from hF9 exon 2 is present to allow efficient splicing of the hIDS transcript into the mature mRNA from the albumin locus, and is effective with both types of the donor integration mechanisms (e.g. NHEJ or HDR). In some embodiments, the donor is the donor designated SB-IDS AAV (e.g. Table 3, SEQ ID NO:17).

In some embodiments, the amount of total urine GAGs are stabilized or reduced in a subject by the methods and compositions disclosed herein as compared to the amount of total urine GAGs in the subject prior to treatment or as compared to total urine GAGs in a patient that has not been treated. In some embodiments, the total urine GAGs are reduced 5%, 10%, 15%, 20%, 25%, 30%, 35%, 40%, 45%, 50%, 55%, 60%, 65%, 70%, 75%, 80%, 85%, 90%, 95% or 100%, or any value there between. In some embodiments, the amount of urine dermatan sulfate GAGs are stabilized or reduced in a subject by the methods and compositions disclosed herein as compared to the amount of urine dermatan sulfate GAGs in the subject prior to treatment or as compared to urine dermatan sulfate GAGs in a patient that has not been treated. In some embodiments, the urine dermatan sulfate GAGs are reduced 5%, 10%, 15%, 20%, 25%, 30%, 35%, 40%, 45%, 50%, 55%, 60%, 65%, 70%, 75%, 80%, 85%, 90%, 95% or 100%, or any value there between. In some embodiments, the amount of urine heparan sulfate GAGs are stabilized or reduced in a subject by the methods and compositions disclosed herein as compared to the amount of urine heparan sulfate GAGs in the subject prior to treatment or as compared to urine heparan sulfate GAGs in a patient that has not been treated. In some embodiments, the urine heparan sulfate GAGs are reduced 5%, 10%, 15%, 20%, 25%, 30%, 35%, 40%, 45%, 50%, 55%, 60%, 65%, 70%, 75%, 80%, 85%, 90%, 95% or 100%, or any value there between. In some embodiments, GAG levels are used as a biochemical marker to assess treatment effect once a patient has withdrawn from ERT following treatment with the compositions disclosed herein. GAG measurements are most useful when used in conjunction with an assessment of other clinical parameters for the patient.

In some embodiments, the ZFNs useful in the methods and compositions disclosed herein in the albumin-specific pair are similarly delivered (e.g. to the hepatocytes) via AAV2/6 delivery wherein one AAV comprises the left ZFN (e.g. SBS-47171 or SB-71557; SEQ ID NO:9 or SEQ ID NO:30, respectively) and another comprises the right ZFN (e.g. SBS-47898 or SB-71728; SEQ ID NO:12 or SEQ ID NO:31, respectively). In some embodiments, ZFN expression is under control, for example, by a liver-specific enhancer and promoter, comprised of the human ApoE enhancer and human α1-anti-trypsin (hAAT) promoter (Miao C H et al. (2000) Mol. Ther. 1(6):522-532 (200)). In some embodiments, ZFN expression is under the minimal transthyretin promoter. In some embodiments, the expression cassette comprising a ZFN comprises one or more FLAG tags (e.g. a N-terminal peptide), a nuclear localization sequence (NLS), a WPRE sequence, an alternate poly A sequence, a 5′ UTR and/or a 3′ UTR as described above. In some embodiments, the ApoE/hAAT promoter (e.g. SEQ ID NO:2) is specifically and highly active (e.g. in hepatocytes, the intended target tissue), but is inactive in non-liver cell and tissue types; this prevents ZFN expression and activity in non-target tissues. In some embodiments, ZFN expression is under the minimal transthyretin promoter. In some embodiments, the expression cassette comprising a ZFN comprises one or more FLAG tags, a nuclear localization sequence (NLS), a WPRE sequence, an alternate poly A sequence, a 5′ UTR and/or a 3′ UTR as described above. In some embodiments, the ZFNs and IDS donor are delivered, for example, using a composition comprising all three components: two AAV vectors for each component of a paired ZFN and 1 AAV carrying the donor (e.g., a composition which comprises SB-47171 AAV or SB-71557 AAV (e.g. Table 1 or Table 4), SB-47898 AAV or SB-71728 AAV (e.g. Table 2 or Table 5) and SB-IDS AAV (e.g. Table 3)).

In some embodiments, reduced, stabilized or eliminated urine GAGs (e.g. urine GAG levels) is measured in the subject's urine after treatment with the methods and compositions disclosed herein. In some embodiments, reduced, stabilized or eliminated GAGs in the urine (for example urine GAG levels, heparan sulfate GAGs, and/or dermatan sulfate GAGs) is measured by any method known in the art. Exemplary methods to measure urine GAGs include the Dimethyl Methylene Blue (DMB) assay (see e.g. de Jong et al. (1989) Clin Chem 35/7:1472-1479); a method dependent on serine proteases and a labeled substrate for the serine protease, an inhibitor of the serine protease, and a urine sample suspected of comprising one or more glycosaminoglycans (see e.g. U .S. Patent Publication No. 2013/0189718); a multiplex assay (Langereis et al. (2015) PLoS One 10(9):e0138622) based on enzymatic digestion the of heparan sulfate (HS), dermatan sulfate (DS) and keratan sulfate (KS) found in the urine, followed by quantification by LC-MS/MS; and an assay that can be used to determine the concentration of specific types of GAGs that utilizes a RapidFire (RF, Agilent) high-throughput mass spectrometry system (see Tomatsu et al. (2014) J Anal Bioanal Tech. Mar. 1; 2014 (Suppl 2):006).

In some embodiments, the subject has received ERT at baseline or has received ERT in the past, while in other embodiments, the subject has not received ERT.

In some embodiments, the treatment using the methods and compositions as disclosed herein of the subject comprises dosing of a composition of the invention, for example, via a peripheral vein catheter. In some embodiments, the composition is added to a normal saline (NS) or phosphate buffered saline (PBS) diluent, wherein the diluent further comprises, for example, human serum albumin. In some embodiments, the subject receives a total AAV dose, for example, of 5e12 vg/kg comprising 5e11 vg/kg of each ZFN AAV2/6 comprising either a left ZFN or a right ZFN, and 4e12 vg/kg of the hIDS donor AAV as disclosed herein. In some embodiments, the subject receives a total AAV dose, for example, of 1e13 vg/kg comprising 1e12 vg/kg of each ZFN AAV2/6 comprising either a left ZFN or a right ZFN, and 8e12 vg/kg of the hIDS donor AAV as disclosed herein. In some embodiments, the subject receives a total AAV dose, for example, of 5e13 vg/kg comprising 5e12 vg/kg of each ZFN AAV comprising either a left ZFN or a right ZFN, and 4e13 of the hIDS donor AAV as disclosed herein. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. In some embodiments, the components may be administered separately, or, preferably a composition comprising all components (paired ZFNs on the same or different vectors and IDS donor), for example a composition which comprises SB-47171 AAV (e.g. Table 1), SB-47898 AAV (e.g., Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the reduced, stabilized or eliminated urine GAGs is measured for the subject, for example after a treatment with a composition of the invention at a total dose of 5e12 vg/kg, of 1e13 vg/kg, of 5e13 vg/kg, of 1e14 vg/kg, of 5e14 vg/kg and/or 1e15 vg/kg. In some embodiments, the reduced, stabilized or eliminated urine GAGs is measured for the subject after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In some embodiments, provided herein is a method of improving, delaying a decline or maintaining the functional ability in a subject with MPS II by treating the subject with a standard dosing regimen, for example, of ERT in combination with treatment with a composition of the invention as disclosed herein, as compared with a subject that has not been treated, the method comprising administering to the subject an effective amount of hIDS transgene and zinc finger nucleases (ZFN) and with a standard ERT dose, wherein the subject has, for example, an improvement in functional ability, a delay in decline or maintenance of functional ability after treatment. In some embodiments, the hIDS transgene (e.g. SEQ ID NO:15) is delivered to the hepatocyte via AAV2/6 delivery, and the hIDS delivery vector further comprises homology arms (e.g. SEQ ID NO:13 and SEQ ID NO:16) flanking the hIDS transgene with specificity for the regions flanking the ZFN cut site in the albumin locus. In some embodiments, the left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site. In some embodiments, the arms of homology are used, for example, to help facilitate targeted integration of the hIDS transgene at the albumin intron 1 locus via homology directed repair. In some embodiments, the size of the homology arms are chosen, for example, to avoid repetitive sequences and splicing elements in the albumin locus that can inhibit targeted integration or transgene expression. In some embodiments, the polyA sequences are derived from the bovine growth hormone gene. In some embodiments, the hIDS transgene donor further comprises a stop codon, for example, at the 3′ end to prevent further transcription of the albumin sequences into which the IDS transgene is inserted. In some embodiments, the rAAV2/6 donor vector containing the human IDS transgene (e.g. SB-IDS donor) is a promoterless construct, for example, that comprises a partial IDS cDNA comprising parts of exon 1 plus exons 2-9 (e.g. SEQ ID NO:15). In some embodiments, the splice acceptor site (e.g. SA, SEQ ID NO:14) derived, for example, from hF9 exon 2 is present to allow efficient splicing of hIDS transgene into the mature mRNA from the albumin locus, and is effective with both types of the donor integration mechanisms (e.g. NHEJ or HDR). In some embodiments, the donor is the donor designated SB-IDS AAV (e.g. Table 3, SEQ ID NO:17).

In some embodiments, the ZFNs in the albumin-specific pair are similarly delivered (e.g. to the hepatocytes) via AAV2/6 delivery wherein one AAV comprises the left ZFN (e.g. SBS-47171 or SB-71557; SEQ ID NO:9 or SEQ ID NO:30, respectively) and another comprises the right ZFN (e.g. SBS-47898 or SB-71728; SEQ ID NO:12 or SEQ ID NO:31, respectively). In some embodiments, ZFN expression is under control, for example, by a liver-specific enhancer and promoter, comprised of the human ApoE enhancer and human α1-anti-trypsin (hAAT) promoter (Miao C H et al. (2000) Mol. Ther. 1(6):522-532 (200)). In some embodiments, ZFN expression is under the minimal transthyretin promoter. In some embodiments, the expression cassette comprising a ZFN comprises one or more FLAG tags, a nuclear localization sequence (NLS), a WPRE sequence, an alternate poly A sequence, a 5′ UTR or a 3′ UTR as described above. In some embodiments, the ApoE/hAAT promoter (e.g. SEQ ID NO:2) is specifically and highly active (e.g. in hepatocytes, the intended target tissue), but is inactive in non-liver cell and tissue types; this prevents ZFN expression and activity in non-target tissues.

In some embodiments, improvement in, delay in decline or maintenance of functional ability after treatment with the methods and compositions disclosed herein, is measured in the subject after treatment. In some embodiments, an improvement in, delay in decline or maintenance of functional ability is measured, for example, by a change from baseline in forced vital capacity measured by a pulmonary function test. In some embodiments, an improvement in, delay in decline or maintenance of functional ability is measured, for example, by a change from base line in distance walked measured by a 6-minute walk test. In some embodiments, the improvement in, delay in decline or maintenance of functional ability is measured, for example, by a change from baseline in joint range of motion. In some embodiments, the improvement in, delay in decline or maintenance of functional ability is measured, for example, by a change from baseline in neurocognitive abilities as measured by WASI-II (Wechsler Abbreviated Scale of Intelligence, Second Edition (Shapiro et al., ibid)), WPPSI-IV (Wechsler Preschool and Primary Scale of Intelligence), or BSID-III (Bayley Scales of Infant Development), and by VABS-II (Vineland Adaptive Behavior Scales).

In some embodiments, the subject has received ERT at baseline or has received ERT in the past, while in other embodiments, the subject has not received ERT.

In some embodiments, the treatment comprises dosing of a composition of the invention (e.g. via a peripheral vein catheter). In some embodiments, the composition is added to a normal saline (NS) or phosphate buffered saline (PBS) diluent, wherein the diluent further comprises human serum albumin. In some embodiments, the subject receives a total AAV dose, for example, of 5e12 vg/kg comprising 5e11 vg/kg of each ZFN AAV2/6 comprising either a left ZFN or a right ZFN, and 4e12 vg/kg of the hIDS donor AAV as disclosed herein. In other embodiments, the subject receives a total AAV dose, for example, of 1e13 vg/kg comprising 1e12 vg/kg of each ZFN AAV2/6 comprising either a left ZFN or a right ZFN, and 8e12 vg/kg of the hIDS donor AAV as disclosed herein. In some embodiments, the subject receives a total AAV dose, for example, of 5e13 vg/kg comprising 5e12 vg/kg of each ZFN AAV comprising either a left ZFN or a right ZFN, and 4e13 of the hIDS donor AAV as disclosed herein. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. In some embodiment the components may be administered separately, or, preferably a composition comprising all components (paired ZFNs on the same or different vectors and IDS donor), for example a composition which comprises SB-47171 AAV (e.g. Table 1), SB-47898 AAV (e.g. Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the improvement in, delay in decline or maintenance of function ability is measured for the subject, for example, after a treatment with a composition of the invention at a total dose of 5e12 vg/kg, of 1e13 vg/kg, of 5e13 vg/kg, of 1e14 vg/kg, of 5e14 vg/kg and/or 1e15 vg/kg. In some embodiments, the improvement in, delay in decline, or maintenance of functional ability is measured for the subject after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In some embodiments, provided herein is a method of suppressing or delaying disability progression in a human subject having MPS II as compared with a subject that has not been treated with the methods and compositions of the invention, the method comprising administering to the subject an effective amount of hIDS transgene and zinc finger nucleases (ZFN) wherein the subject has a stabilization, suppression or delay in disability progression after treatment with the methods and compositions as disclosed herein. In some embodiment, the hIDS transgene (e.g. SEQ ID NO:15) is delivered (e.g. to the hepatocyte) via AAV2/6 delivery, and the hIDS delivery vector further comprises homology arms (e.g. SEQ ID NO:13 and SEQ ID NO:16) flanking the hIDS transgene that have specificity for the regions flanking the ZFN cut site in the albumin locus. In some embodiments, the left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site. In some embodiments, the arms of homology are used, for example, to help facilitate targeted integration of the hIDS transgene at the albumin intron 1 locus via homology directed repair. In some embodiments, the size of the homology arms were chosen, for example, to avoid repetitive sequences and splicing elements in the albumin locus that can inhibit targeted integration or transgene expression. In some embodiments, the polyA sequences are derived from the bovine growth hormone gene. In some embodiments, the hIDS transgene donor further comprises, for example, a stop codon at the 3′ end to prevent further transcription of the albumin sequences into which the IDS transgene is inserted. In some embodiments, the rAAV2/6 donor vector containing the human IDS transgene (e.g. SB-IDS donor) is a promoterless construct that comprises, for example, a partial IDS cDNA comprising parts of exon 1 plus exons 2-9 (e.g. SEQ ID NO:15). In some embodiments, the splice acceptor site (e.g. SA, SEQ ID NO:14) derived, for example, from hF9 exon 2 is present to allow efficient splicing of the hIDS transcript into the mature mRNA from the albumin locus, and is effective with both types of the donor integration mechanisms (NHEJ or HDR). In some embodiments, the donor is the donor designated SB-IDS AAV (e.g. Table 3, SEQ ID NO:17).

In some embodiments, the ZFNs in the albumin-specific pair are similarly delivered (e.g. to the hepatocytes) via AAV2/6 delivery wherein one AAV comprises the left ZFN (e.g. SBS-47171 or SB-71557; SEQ ID NO:9 or SEQ ID NO:30, respectively) and another comprises the right ZFN (e.g. SBS-47898 or SB-71728; SEQ ID NO:12 or SEQ ID NO:31, respectively). In some embodiments, ZFN expression is controlled by a liver-specific enhancer and promoter, for example, comprised of the human ApoE enhancer and human α1-anti-trypsin (hAAT) promoter (Miao C H et al. (2000) Mol. Ther. 1(6):522-532 (200)). In some embodiments, ZFN expression is under the minimal transthyretin promoter. In some embodiments, the expression cassette comprising a ZFN comprises one or more FLAG tags, a nuclear localization sequence (NLS), a WPRE sequence, an alternate poly A sequence, a 5′ UTR or a 3′ UTR as described above. In some embodiments, the ApoE/hAAT promoter (e.g., SEQ ID NO:2) is specifically and highly active in hepatocytes, the intended target tissue, but is inactive in non-liver cell and tissue types; this prevents ZFN expression and activity in non-target tissues.

In some embodiments, stabilization, suppression or delay of disability progression is measured in the subject after treatment with the methods and compositions as disclosed herein. In some embodiments, stabilization, suppression or delay of disability progression is measured, for example, by a change from baseline or stabilization in forced vital capacity measured by a pulmonary function test. In some embodiments, stabilization, suppression or delay of disability progression is measured, for example, by a change from base line or stabilization in distance walked measured by a 6-minute walk test. In some embodiments, stabilization, suppression or delay of disability progression is measured, for example, by a change from baseline or stabilization in joint range of motion (JROM). In some embodiments stabilization, suppression or delay of disability progression is measured, for example, by a change from baseline or stabilization in neurocognitive abilities as measured by WASI-II (Wechsler Abbreviated Scale of Intelligence, Second Edition (Shapiro et al., ibid)), WPPSI-IV (Wechsler Preschool and Primary Scale of Intelligence), or BSID-III (Bayley Scales of Infant Development), and by VABS-II (Vineland Adaptive Behavior Scales).

In some embodiments, the subject has received ERT at baseline or has received ERT in the past, while in other embodiments, the subject has not received ERT.

In some embodiments, the treatment comprises dosing of a composition of the invention (e.g. via a peripheral vein catheter). In some embodiments, the composition is added to a normal saline (NS) or phosphate buffered saline (PBS) diluent. In some embodiments, the subject receives a total AAV dose, for example of 5e12 vg/kg comprising 5e11 vg/kg of each ZFN AAV2/6 comprising either a left ZFN or a right ZFN, and 4e12 vg/kg of the hIDS donor AAV as disclosed herein. In some embodiments, the subject receives a total AAV dose, for example of 1e13 vg/kg comprising 1e12 vg/kg of each ZFN AAV2/6 comprising either a left ZFN or a right ZFN, and 8e12 vg/kg of the hIDS donor AAV as disclosed herein. In some embodiments, the subject receives a total AAV dose, for example, of 5e13 vg/kg comprising 5e12 vg/kg of each ZFN AAV comprising either a left ZFN or a right ZFN, and 4e13 of the hIDS donor AAV as disclosed herein. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. In some embodiments the components may be administered separately, or, preferably a composition comprising all components (paired ZFNs on the same or different vectors and IDS donor), for example a composition which comprises SB-47171 AAV (e.g. Table 1), SB-47898 AAV (e.g. Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the delayed need for ERT is measured for the subject after treatment with a composition of the invention via a total dose of 5e12 vg/kg, of 1e13 vg/kg, of 5e13 vg/kg of 1e14 vg/kg, of 5e14 vg/kg and/or 1e15 vg/kg. In some embodiments, the delayed need for ERT measured for the subject after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In some embodiments, provided herein is a method of stabilizing, delaying, reducing or preventing the need for the use of a medical ventilator device in a subject with MPS II as compared with a subject that has not been treated with the methods and compositions as disclosed herein, the method comprising administering to the subject an effective amount of hIDS transgene and zinc finger nucleases (ZFN) wherein the subject has a delay, reduction or prevention of the need for the use of a medical ventilator device. In some embodiments, the hIDS transgene (SEQ ID NO:15) is delivered (e.g. to the hepatocyte) via AAV2/6 delivery, and the hIDS delivery vector further comprises homology arms (e.g. SEQ ID NO:13 and SEQ ID NO:16) flanking the hIDS transgene with specificity for the regions flanking the ZFN cut site in the albumin locus. In some embodiments, the left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site. In some embodiments, the arms of homology are used to help facilitate targeted integration of the hIDS transgene at the albumin intron 1 locus via homology directed repair. In some embodiments, the size of the homology arms were chosen to avoid repetitive sequences and splicing elements in the albumin locus that can inhibit targeted integration or transgene expression. In some embodiments, the polyA sequences are derived from the bovine growth hormone gene. In some embodiments, the hIDS transgene donor further comprises a stop codon at the 3′ end to prevent further transcription of the albumin sequences into which the IDS transgene is inserted. In some embodiments, the rAAV2/6 donor vector containing the human IDS transgene (e.g. SB-IDS donor) is a promoterless construct that comprises a partial IDS cDNA comprising parts of exon 1 plus exons 2-9 (e.g. SEQ ID NO:15). The splice acceptor site (e.g. SA, SEQ ID NO:14) derived from hF9 exon 2 is present to allow efficient splicing of hIDS transgene into the mature mRNA from the albumin locus, and is effective with both types of the donor integration mechanisms (e.g. NHEJ or HDR). In some embodiments, the donor is the donor designated SB-IDS AAV (e.g. Table 3, SEQ ID NO:17).

In some embodiments, the ZFNs in the albumin-specific pair are similarly delivered to the hepatocytes via AAV2/6 delivery wherein one AAV comprises the left ZFN (e.g. SBS-47171 or SB-71557; SEQ ID NO:9 or SEQ ID NO:30, respectively) and another comprises the right ZFN (e.g. SB S-47898 or SB-71728; SEQ ID NO:12 or SEQ ID NO:31, respectively). In some embodiments, ZFN expression is controlled by a liver-specific enhancer and promoter, comprised of the human ApoE enhancer and human α1-anti-trypsin (hAAT) promoter (Miao C H et al. (2000) Mol. Ther. 1(6):522-532 (200)). In some embodiments, ZFN expression is under the minimal transthyretin promoter. In some embodiments, the expression cassette comprising a ZFN comprises one or more FLAG tags, a nuclear localization sequence (NLS), a WPRE sequence, an alternate poly A sequence, a 5′ UTR or a 3′ UTR as described above. In some embodiments, the ApoE/hAAT promoter (e.g. SEQ ID NO:2) is specifically and highly active in hepatocytes, the intended target tissue, but is inactive in non-liver cell and tissue types; this prevents ZFN expression and activity in non-target tissues. In some embodiments, the stabilized, delayed, reduced or prevented need for the use of a ventilator is measured in the subject after treatment. In some embodiments, the stabilized, delayed, reduced or prevented need for use of a ventilator is measured, for example, by a change from baseline in forced vital capacity measured by a pulmonary function test. In some embodiments, the stabilized, delayed, reduced or prevented need for use of a ventilator is measured, for example, by a change from base line in distance walked measured by a 6-minute walk test.

In some embodiments, the treatment using the methods and compositions as disclosed herein comprises dosing of with a composition of the invention (e.g. via a peripheral vein catheter). In some embodiments, the composition is added to a normal saline (NS) or phosphate buffered saline (PBS) diluent, wherein the diluent further comprises human serum albumin. In some embodiments, the subject receives a total AAV dose, for example of 5e12 vg/kg comprising 5e11 vg/kg of each ZFN AAV2/6 comprising either a left ZFN or a right ZFN, and 4e12 vg/kg of the hIDS donor AAV as disclosed herein. In other embodiments, the subject receives a total AAV dose, for example of 1e13 vg/kg comprising 1e12 vg/kg of each ZFN AAV2/6 comprising either a left ZFN or a right ZFN, and 8e12 vg/kg of the hIDS donor AAV as disclosed herein. In some embodiments, the subject receives a total AAV dose, for example, of 5e13 vg/kg comprising 5e12 vg/kg of each ZFN AAV comprising either a left ZFN or a right ZFN, and 4e13 of the hIDS donor AAV as disclosed herein. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. In some embodiments, the components may be administered separately, or, preferably a composition comprising all components (paired ZFNs on the same or different vectors and IDS donor), for example a composition which comprises SB-47171 AAV (e.g., Table 1), SB-47898 AAV (e.g., Table 2) and SB-IDS AAV (e.g., Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the reduced or delayed need for use of a ventilator is measured for the subject after treatment with a composition of the invention with a total dose of 5e12 vg/kg, of 1e13 vg/kg, of 5e13 vg/kg, of 1e14 vg/kg, of 5e14 vg/kg and/or 1e15 vg/kg. In some embodiments, the reduced or delayed need for use of a ventilator is measured for the subject after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In some embodiments, provided herein is a method of stabilizing, delaying, reducing or preventing the onset of a subject being wheelchair dependent in a human subject having MPS II as compared to a subject that that has not been treated with the methods and compositions as disclosed herein, the method comprising administering to the subject an effective amount of hIDS transgene and zinc finger nucleases (ZFN) wherein the subject has a stabilized, delayed, reduced or prevented onset of being wheelchair dependent after treatment. In some embodiments, the hIDS transgene (e.g. SEQ ID NO:15) is delivered to the hepatocyte via AAV2/6 delivery, and the hIDS delivery vector further comprises homology arms (e.g. SEQ ID NO:13 and SEQ ID NO:16) flanking the hIDS transgene that are specific for the regions flanking the ZFN cut site in the albumin locus. In some embodiments the left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site. In some embodiments, the arms of homology are used to help facilitate targeted integration of the hIDS transgene at the albumin intron 1 locus via homology directed repair. In some embodiments, the size of the homology arms are chosen to avoid repetitive sequences and splicing elements in the albumin locus that can inhibit targeted integration or transgene expression. In some embodiments, the polyA sequences are derived from the bovine growth hormone gene. In some embodiments, the hIDS transgene donor further comprises a stop codon at the 3′ end to prevent further transcription of the albumin sequences into which the IDS transgene is inserted. In some embodiments, the rAAV2/6 donor vector containing the human IDS transgene (e.g. SB-IDS donor) is a promoterless construct that comprises a partial IDS cDNA comprising parts of exon 1 plus exons 2-9 (e.g. SEQ ID NO:15). The splice acceptor site (e.g. SA, SEQ ID NO:14) derived from hF9 exon 2 is present to allow efficient splicing of the hIDS transcript into the mature mRNA from the albumin locus, and is effective with both types of the donor integration mechanisms (e.g. NHEJ or HDR). In certain embodiments, the donor is the donor designated SB-IDS AAV (e.g. Table 3, SEQ ID NO:17).

In some embodiments, the ZFNs in the albumin-specific pair are similarly delivered to the hepatocytes via AAV2/6 delivery wherein one AAV comprises the left ZFN (e.g. SBS-47171 or SB-71557; SEQ ID NO:9 or SEQ ID NO:30, respectively) and another comprises the right ZFN (e.g. SB S-47898 or SB-71728; SEQ ID NO:12 or SEQ ID NO:31, respectively). In some embodiments, ZFN expression is controlled by a liver-specific enhancer and promoter, comprised of the human ApoE enhancer and human α1-anti-trypsin (hAAT) promoter (Miao C H et al. (2000) Mol. Ther. 1(6):522-532 (200)). In some embodiments, ZFN expression is under the minimal transthyretin promoter. In some embodiments, the expression cassette comprising a ZFN comprises one or more FLAG tags, a nuclear localization sequence (NLS), a WPRE sequence, an alternate poly A sequence, a 5′ UTR or a 3′ UTR as described above. In some embodiments, the ApoE/hAAT promoter (e.g. SEQ ID NO:2) is specifically and highly active in hepatocytes, the intended target tissue, but is inactive in non-liver cell and tissue types; this prevents ZFN expression and activity in non-target tissues.

In some embodiments, stabilized, delayed, reduced or the prevention of the onset of being wheelchair dependent is measured in the subject after treatment. In some embodiments, stabilized, delayed, reduced or prevention of the onset of being wheelchair dependent is measured by a change from baseline in forced vital capacity measured by a pulmonary function test. In some embodiments, stabilized, delayed, reduced or prevention of the onset of being wheelchair dependent is measured by a change from base line or stabilization in distance walked measured by a 6-minute walk test. In some embodiments, stabilized, delayed, reduced or prevention of onset of being wheelchair dependent is measured by a change from baseline or stabilization in joint range of motion. In some embodiments stabilization, delay, reduction or prevention of the onset of being wheelchair dependent is measured by WASI-II (Wechsler Abbreviated Scale of Intelligence, Second Edition (Shapiro et al., ibid)), WPPSI-IV (Wechsler Preschool and Primary Scale of Intelligence), or BSID-III (Bayley Scales of Infant Development), and by VABS-II (Vineland Adaptive Behavior Scales). In some embodiments, stabilization or delaying onset of confirmed disability progression or reducing the risk of confirmed disability progression is measured by a change from baseline or stabilization in total GAG, DS GAG, and HS GAG levels measured in liver tissue and CSF.

In some embodiments, the subject has received ERT at baseline or has received ERT in the past, while in other embodiments, the subject has not received ERT.

In some embodiments, the treatment comprises dosing with a composition of the invention via a peripheral vein catheter. In some embodiments, the composition is added to a normal saline (NS) or phosphate buffered saline (PBS) diluent, wherein the diluent further comprises human serum albumin. In some embodiments, the subject receives a total AAV dose of 5e12 vg/kg comprising 5e11 vg/kg of each ZFN AAV2/6 comprising either a left ZFN or a right ZFN, and 4e12 vg/kg of the hIDS donor AAV. In other embodiments, the subject receives a total AAV dose, for example, of 1e13 vg/kg comprising 1e12 vg/kg of each ZFN AAV2/6 comprising either a left ZFN or a right ZFN, and 8e12 vg/kg of the hIDS donor AAV as disclosed herein. In some embodiments, the subject receives a total AAV dose, for example, of 5e13 vg/kg comprising 5e12 vg/kg of each ZFN AAV comprising either a left ZFN or a right ZFN, and 4e13 of the hIDS donor AAV as disclosed herein. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. In some embodiments, the components may be administered separately, or, preferably a composition comprising all components (e.g. paired ZFNs on the same or different vectors and IDS donor), for example a composition which comprises SB-47171 AAV (e.g. Table 1), SB-47898 AAV (e.g. Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the stabilized, delayed, reduced or the prevention of the onset of being wheelchair dependent is measured for the subject after a total dose of 5e12 vg/kg of the compositions described herein, of 1e13 vg/kg, of 5e13 vg/kg of 1e14 vg/kg, of 5e14 vg/kg and/or 1e15 vg/kg. In some embodiments, the stabilized, delayed, reduced or prevention of the onset of being wheelchair dependent is measured for the subject after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In some embodiments, provided herein is a method of extending life expectancy in a subject with MPS II as compared with a subject that has not been treated with the methods and compositions as disclosed herein, the method comprising administering to the subject an effective amount of hIDS transgene and zinc finger nucleases (ZFN) wherein the subject has an extended life expectancy. In some embodiments, the hIDS transgene (e.g. SEQ ID NO:15) is delivered to the hepatocyte via AAV2/6 delivery, and the hIDS delivery vector further comprises homology arms (e.g. SEQ ID NO:13 and SEQ ID NO:16) flanking the hIDS transgene that are specific for the regions flanking the ZFN cut site in the albumin locus. The left arm of homology (LA) contains about 280 nucleotides (e.g. SEQ ID NO:13) of identical sequence upstream of the albumin intron 1 cleavage site, and the right arm of homology (RA) contains about 100 nucleotides (e.g. SEQ ID NO:16) of identical sequence downstream of the cleavage site. In some embodiments, the arms of homology are used to help facilitate targeted integration of the hIDS transgene at the albumin intron 1 locus via homology directed repair. In some embodiments, the size of the homology arms were chosen to avoid repetitive sequences and splicing elements in the albumin locus that can inhibit targeted integration or transgene expression. In some embodiments, the polyA sequences are derived from the bovine growth hormone gene. In some embodiments, the hIDS transgene donor further comprises a stop codon at the 3′ end to prevent further transcription of the albumin sequences into which the IDS transgene is inserted. In some embodiments, the rAAV2/6 donor vector containing the human IDS transgene (SB-IDS donor) is a promoterless construct that comprises a partial IDS cDNA comprising parts of exon 1 plus exons 2-9 (SEQ ID NO:15). In some embodiments, the splice acceptor site (e.g. SA, SEQ ID NO:14) derived from hF9 exon 2 is present to allow efficient splicing of the hIDS transcript into the mature mRNA from the albumin locus, and is effective with both types of the donor integration mechanisms (e.g. NHEJ or HDR). In certain embodiments, the donor is the donor designated SB-IDS AAV (e.g. Table 3 and sequence following Table 3).

In some embodiments, the ZFNs in the albumin-specific pair are similarly delivered to the hepatocytes via AAV2/6 delivery wherein one AAV comprises the left ZFN (e.g. SBS-47171 or SB-71557; SEQ ID NO:9 or SEQ ID NO:30, respectively) and another comprises the right ZFN (e.g. SB S-47898 or SB-71728; SEQ ID NO:12 or SEQ ID NO:31, respectively). In some embodiments, ZFN expression is controlled by a liver-specific enhancer and promoter, comprised of the human ApoE enhancer and human α1-anti-trypsin (hAAT) promoter (Miao C H et al. (2000) Mol. Ther. 1(6):522-532). In some embodiments, ZFN expression is under the minimal transthyretin promoter. In some embodiments, the expression cassette comprising a ZFN comprises one or more FLAG tags, a nuclear localization sequence (NLS), a WPRE sequence, an alternate poly A sequence, a 5′ UTR or a 3′ UTR as described above. In some embodiments, the ApoE/hAAT promoter (e.g. SEQ ID NO:2) is specifically and highly active in hepatocytes, the intended target tissue, but is inactive in non-liver cell and tissue types; this prevents ZFN expression and activity in non-target tissues. In some embodiments, the extension of life expectancy measured in the subject after treatment.

In some embodiments, the treatment comprises dosing of a composition as disclosed herein via a peripheral vein catheter. In some embodiments, the composition is added to a normal saline (NS) or phosphate buffered saline (PBS) diluent, wherein the diluent further comprises human serum albumin. In some embodiments, the subject receives a total AAV dose, for example, of 5e12 vg/kg comprising 5e11 vg/kg of each ZFN AAV2/6 comprising either a left ZFN or a right ZFN, and 4e12 vg/kg of the hIDS donor AAV as disclosed herein. In some embodiments, the subject receives a total AAV dose, for example, of 1e13 vg/kg comprising 1e12 vg/kg of each ZFN AAV2/6 comprising either a left ZFN or a right ZFN, and 8e12 vg/kg of the hIDS donor AAV as disclosed herein. In some embodiments, the subject receives a total AAV dose, for example, of 5e13 vg/kg comprising 5e12 vg/kg of each ZFN AAV comprising either a left ZFN or a right ZFN, and 4e13 of the hIDS donor AAV as disclosed herein. In some embodiments, the subject receives a total AAV dose, for example, of 1e14 vg/kg comprising 1e13 vg/kg of each ZFN AAV2/6 comprising, for example, either a left ZFN or a right ZFN, and 8e13 vg/kg of the hIDS donor AAV. In some embodiments, the subject receives a total AAV dose, for example, of 5e14 vg/kg comprising 5e13 vg/kg of each ZFN AAV2/6 comprising, for example, either a left ZFN or a right ZFN, and 4e14 vg/kg of the hIDS donor AAV. In some embodiments, the subject receives a total AAV dose, for example, of 1e15 vg/kg comprising 1e14 vg/kg of each ZFN AAV2/6 comprising, for example, either a left ZFN or a right ZFN, and 8e14 vg/kg of the hIDS donor AAV. In some embodiments, the components may be administered separately, or, preferably a composition comprising all components (paired ZFNs on the same or different vectors and IDS donor), for example a composition which comprises SB-47171 AAV (e.g. Table 1), SB-47898 AAV (e.g. Table 2) and SB-IDS AAV (e.g. Table 3). In some embodiments, the composition comprises SB-71557 AAV (e.g. Table 4, SEQ ID NO:30); SB-71728 (e.g. Table 5, SEQ ID NO:31); and SB-IDS AAV (e.g. Table 3, SEQ ID NO:17).

In some embodiments, the extended life expectancy is measured for the subject after treatment with a composition of the invention at a total dose of 5e12 vg/kg, of 1e13 vg/kg, of 5e13 vg/kg, of 1e14 vg/kg, of 5e14 vg/kg and/or 1e15 vg/kg. In some embodiments, the extended life expectancy is measured for the subject after receiving a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg).

In certain embodiments, a) the need for additional therapeutic procedures in a subject having MPS II is decreased or stabilized; b) the symptoms in a subject having MPS II are decreased or stabilized, c) the amount of GAGs in the urine of a subject with MPS II are reduced, stabilized or eliminated; d) the functional ability in a subject having MPS II is improved or stabilized; e) the need for ERT in a subject with MPS II is decreased or stabilized; f) the need for ERT in a subject with MPS II is delayed or stabilized; g) the dose and/or frequency of ERT treatment stabilizes or decreases in a subject with MPS II that is also treated with a composition as disclosed herein, and/or the subject has stabilized or increased functional ability as compared to a MPS-II subject treated with ERT alone; h) the risk of disability progression in a subject with MPS II is stabilized or decreased; i) the onset of confirmed disability progression is stabilized or delayed in a subject treated with a composition of the invention, j) there is a delay in becoming wheelchair dependent or the need for a wheelchair is abolished; k) the need for the use of a mechanical ventilator is stabilized, reduced, delayed or prevented; l) life expectancy in a subject treated with a composition of the invention is expanded as compared to a subject that has not been treated with the composition.

In some embodiments, the subject is premedicated prior to infusion with a composition of the invention. In some embodiments, the subject is premedicated with prednisone or an equivalent corticosteroid the day prior to infusion with the composition. In some embodiments, the subject is premedicated with prednisone or equivalent corticosteroid on the day prior to infusion with the composition and again on the day of infusion. In some embodiments, the subject is premedicated with prednisone or equivalent corticosteroid on the day prior to infusion with the composition, again on the day of infusion, and/or again on day 7, and/or at week 2, and/or week 4, and/or week 6, and/or week 8 up to a maximum duration of week 20.

In some embodiments of the methods described above and herein, the MPS II is the early onset, severe form of the disease with somatic and cognitive involvement, while in other embodiments, the MPS II is the attenuated MPS II characterized by later onset of somatic disease and little or no central nervous system disease. In further embodiments, the MPS II disease is on the continuum between the two. In some embodiments, the subjects are adults while in some embodiments, the subjects are from the pediatric population.

In certain embodiments according to (or as applied to) any of the embodiments above, the subject is selected for treatment based on having the early onset, severe form of MPS II, while in other embodiments, the subject has the attenuated MPS II characterized by a later onset of somatic disease with little or no central nervous system disease, while in some embodiments, the subject is selected for treatment based on having MPS II disease that is on the continuum between the two.

In some embodiments of the methods described above and herein, a composition of the invention is administered at a total dose of 5e12 vg/kg, of 1e13 vg/kg, of 5e13 vg/kg, of 1e14 vg/kg, of 5e14 vg/kg and/or 1e15 vg/kg. In some embodiments of the method described above and herein, a composition of the invention is administered at a total dose of between 5e12 vg/kg to 1e15 vg/kg (for example, between 5e12 vg/kg and 5e13 vg/kg, between 5e12 vg/kg and 1e14 vg/kg, between 5e12 vg/kg and 5e14 vg/kg and/or between 5e12 vg/kg and 1e15 vg/kg). In some embodiments of the methods described above and herein, the composition is administered intravenously.

In certain embodiments of any of the methods above or herein, a stabilization, reduction or decrease or improvement after administration of a composition of the invention can be compared to a baseline level, to a level in untreated subject(s) and/or to a level in subject(s) receiving a different treatment (such as ERT). In some embodiments, a reduction or decrease or improvement after administration of the composition can be compared to a level in subject(s) receiving Elaprase®.

In another aspect, provided herein is an article of manufacture comprising one or more of the compositions described herein. In certain embodiments, the article of manufacture comprises a formulation that includes three pharmaceutical compositions (e.g., in different containers such as vials) as described herein: a first pharmaceutical composition comprising one member of a ZFN pair (e.g., left ZFN); a second pharmaceutical composition comprising the second member of the ZFN pair (e.g., right ZFN); and a third pharmaceutical composition comprising an IDS donor (e.g., AAV IDS donor). Any concentration of the components can be used, including but not limited to the concentrations shown in Table 6. Further, any ratio of the three pharmaceutical compositions can be used, for example 1:1:8 (left ZFN:right ZFN:IDS donor). The different components may be labeled in any way, for example with different colors used for each composition. In certain embodiments, the article of manufacture comprises: a set of drug product vials comprising i) the ZFN1 vector drug product (SB-A6P-ZLEFT), optionally in a container (e.g., vial) comprising an aluminum flip-top seal having a first color (e.g., white); ii) the ZFN 2 vector drug product (SB-A6P-ZRIGHT), optionally in a container (e.g., vial) comprising an aluminum flip-top seal having a second color different from the first color (e.g., blue); and iii) the third vector SB-A6P-HRL drug product, encoding a DNA repair template encoding a promotorless IDS transgene, optionally in a container (e.g., vial) comprising a third color different from the first and second colors (e.g., orange) aluminum flip-top seal. In further embodiments, a set of drug products comprising AAV vectors encoding SB-71557 (SB-A6P-ZL2) or SB-71728 (SB-A6P-ZR2) and SB-A6P-HRL vector is provided. In any of the compositions described herein, the purified lots of recombinant vector may be formulated in phosphate buffered saline (PBS) containing CaCl₂, MgCl₂, NaCl, Sucrose and poloxamer 188 filled at volumes of 5 mL into glass drug product vials, b) a package insert with instructions for treating MPS II in a subject according to any one of the methods described above and herein. In some embodiments, the composition comprises phosphate buffered saline (PBS) comprising approximately 1.15 mg/ML of sodium phosphate, 0.2 mg/mL potassium phosphate, 8.0 mg/mL sodium chloride, 0.2 mg/mL potassium chloride, 0.13 mg/mL calcium chloride, and 0.1 mg/mL Magnesium chloride. The PBS is further modified with 2.05 mg/mL sodium chloride, 10-12 mg/mL of sucrose and 0.5 to 0 mg/mL of Kolliphor® (poloxamer or P188). The article of manufacture (drug product) is administered (e.g., intravenously) to a subject in need thereof such that IDS is expressed in the subject, including at therapeutic levels for treatment of MPS II at any concentration suitable for the subject (e.g., determined based on weight as described herein). Administration may be one-time or multiple times at any frequency. In addition, the set of drug products may be administered separately or may be combined prior to administration, for example in an intravenous infusion bag.

In another aspect, a method of determining the dose of compositions (e.g., to form an article of manufacture/set of drug products) as described herein for a selected subject is provided, the method comprising: determining the subject's weight (rounded to two decimal points) before treatment (baseline); dividing the subject's weight by the vg/mL concentration to determine the dose to be used. For example, for a 50 kg subject to be treated at Cohort 1, 0.5e14 vg of ZFN1 (e.g. 47171 or 71557), 0.5e14 vg of ZFN2 (e.g. 47898 or 71729) and 4e14 SB-IDS are used. Further, these steps are carried out: (i) Calculate the three product component volumes by multiplying the cohort dose by the patient weight at Baseline and then dividing by the VG concentration, for example as follows: (a). Obtain the cohort and patient weight at Baseline from the study coordinator (b). Obtain the VG concentrations from the Clinical Certificates of Analysis. (ii) Calculate the total volume by adding together the three product component volumes and the NS/PBS volume. (iii) Calculate the volume of HSA intravenous solution required to achieve a final concentration of 0.25% HSA, and (iv) Calculate the adjusted NS/PBS volume. The methods may further comprise providing a formulation (e.g., including an article of manufacture comprising three drug products as described herein) with the correct dosage for the subject's weight, by determining a total volume; and calculating the volume of human serum albumin (HSA) intravenous solution needed, thereby achieving the correct component concentration for the selected subject.

In some embodiments, the dose is determined by volume of the liver of the subject. Weight of a subject does not always directly correlate with liver volume, especially in heavier patients. In pediatric patients less than 2 months of age, optimal dosage of different therapeutics can be based on liver volume to avoid hepatic toxicity (see Bartelink et al. (2006) Clin Pharm 45(11):1077-1097). Thus, for some subjects, dose may be determined by approximate liver volume. In these instances, liver volume may be estimated by methods known in the art, for example by use of formulas based on a combination of parameters such as age, gender, body weight, body height, body mass index and body surface area (Yuan et al. (2008) Transplant Proc 40(10):3536-40). Other methods for estimating or determining of liver volume known in the art include CT or MRI scans and estimations of abdominal geometry (Yang et al. (2018) Yonsei Med J 59(4):546-553; Huynh et al. (2014) AJR Am J Roentgenol 202(1):152-59).

In another aspect, provided herein is a method of administering a composition as described herein, the method comprising providing an article of manufacture as described herein (e.g., a drug product comprising three (AAV) pharmaceutical compositions (left ZFN, right ZFN, AAV donor) separately or together as described herein), formulating one or more intravenous solutions at a selected dose for a subject (e.g., using the methods described herein) and intravenously administering the intravenous solution to the subject in need thereof. In certain embodiments, the three components (ZFN1, ZFN2 and IDS donor) of the article of manufacture are added separately to an approximately 200 mL IV infusion bag, for example an IV infusion bag containing 0.25% HSA in NS or PBS. Total infusion volumes are calculated according to the subject's cohort assignment and body weight (kg) and are expected to be between approximately 240-800 mL depending on subject's cohort assignment and body weight (kg). The prepared infusion product will be administered via intravenous infusion at 100 mL/hour using a constant rate infusion pump, while the subject is in the hospital or acute care facility. Any of the methods described herein may be delivered using an infusion pump, at any rate, for example, 10 to 200 mL/hour (or any value therebetween). In certain embodiments, the intravenous solution is delivered at a rate of 100 mL/hour. Subjects may be receiving ERT or received ERT in the past. In certain embodiments, ERT not given during the week of infusion of the intravenous solution.

Also provided are methods of increasing levels (activity) of IDS in leukocytes of a subject, the methods comprising administering an intravenous solution as described herein (e.g., a system comprising three pharmaceutical compositions). In certain embodiments, the IDS levels are increased from below normal (in MPS II subjects) to levels in the normal range (levels in non-MPS II subjects). Increased IDS levels/activity can be determined by measuring IDS levels/activity directly and/or measuring GAG levels. IDS levels (activity) in plasma and urine may also be increased using the methods and compositions described herein.

In any of the methods described herein, the subject may receive a corticosteroid (e.g., prednisone), for example 1, 2, 3, 4, 5, 6, 7 or more days before infusion of the intravenous, the day of infusion and/or up to 20 or more weeks after infusion, wherein the dosage is determined based on the subject's weight. An exemplary schedule of oral prednisone tapering dose over time determined by the subject's weight is shown below in Table A:

TABLE A
Tapering steroid dose

| | Oral Prednisone (mg/day) | | | | | |
|---|---|---|---|---|---|---|
| Weight of | Day −2 to | Week | | | Week | |
| subject | Day 1 | 1 | Week 2 | Week 3-16 | 17-19 | Week 20 |
| ≥60 | 60 | 60 | 30 | 15 | 5 | STOP |
| 55 | 60 | 60 | 30 | 15 | 5 | STOP |
| 50 | 50 | 50 | 25 | 15 | 5 | STOP |
| 45 | 45 | 45 | 25 | 15 | 5 | STOP |
| 40 | 40 | 40 | 20 | 10 | 5 | STOP |
| 35 | 35 | 35 | 20 | 10 | 5 | STOP |
| 30 | 30 | 30 | 15 | 10 | 5 | STOP |

In some embodiments, other doses (including higher or lower) of corticosteroid or other immunosuppressants may be used (e.g. 2.0, 1.5 mg/kg/day of prednisolone or more, or methotrexate at 7.5-15.5 mg/week) than those exemplified in Table A. In some embodiments, initiation of the taper occurs later (for example, at 4, 5, 6, 7, or 8 or more weeks) than exemplified in Table A.

These and other aspects will be readily apparent to the skilled artisan in light of disclosure as a whole.

### BRIEF DESCRIPTION OF THE DRAWINGS

FIG. 1 is schematic showing the steps taken to devise a PK/PD model to estimate the minimally effective dose needed to alleviate the symptoms of MPS II in a subject.

FIG. 2 is a schematic showing the factors considered in devising the model and the mathematical calculations used to link the Elaprase® human PK data to the observed SB-913 mouse data.

FIG. 3 shows the alignment of the predicted pharmacokinetic (PK, IDS plasma activity) and the predicted pharmacokinetic dynamic (PD, urine GAG concentration) data from the mouse studies wherein the mice have been treated with the polynucleotides of the invention. The predicted values are shown as solid black lines while the observed values are shown as open circles. The data demonstrates that the predicted and observed values align.

FIG. 4 shows the predicted PK and PD values in humans for the three different dose groups. The model predicts that even the lowest dose of 5 e+12 will have efficacy in reducing urine GAGs.

FIGS. 5A and 5B are diagrams depicting the breakdown of glucosaminoglycans (GAGs). FIG. 5A shows the catabolic breakdown of dermatan sulfate. FIG. 5B shows the catabolic breakdown of heparan sulfate. MPS II disease results in the inability to initiate the process of breaking down both dermatan sulfate and heparan sulfate, leading to the accumulation of these GAGs in nearly all organs and body tissues and in the urine of a subject with MPS II. Chronic accumulation of GAGs inside cellular lysosomes results in cellular engorgement, organomegaly, tissue destruction, and organ system dysfunction in MPS II patients.

FIGS. 6A through 6C are graphs depicting the percent change in urine GAG, urine dermatan sulfate and urine heparan sulfate levels at 16 weeks post treatment. FIG. 6A shows the percent change from baseline in urine total GAG in cohort 1 (left panel), where the triangles show the data points for subject #1 and the circles show the data points for subject #2 and in cohort 2 (right panel), where the diamonds show the data points for subject #3 and the squares show the data points for subject #4. Total urine GAG was measured by a validated 1,9-dimethylene blue (DMB) colorimetric assay. FIG. 6B shows the percent change from baseline in urine dermatan sulfate in cohort 1 (left panel), where the triangles show the data points for subject #1 and the circles show the data points for subject #2 and in cohort 2 (right panel), where the diamonds show the data points for subject #3 and the squares show the data points for subject #4. FIG. 6C depicts the percent change from baseline in urine heparan sulfate in cohort 1 (left panel), where the triangles show the data points for subject #1 and the circles show the data points for subject #2 and in cohort 2 (right panel), where the diamonds show the data points for subject #3 and the squares show the data points for subject #4. Urine dermatan sulfate and heparan sulfate were measured by a validated MS/MS assay. The data in FIG. 6 shows that the subjects in cohort 2 had decreased total urine GAG, dermatan sulfate and heparan sulfate.

FIG. 7 is a schematic illustrating the assay used to detect integration of the IDS transgene into the albumin locus in the liver. The assay is a PCR-based assay dependent on using primer sets that will detect a unique albumin Exon1-IDS fusion in mRNA expressed in the liver. In the presence of the inserted transgene, the PCR product is driven by one primer that corresponds to the albumin gene, and one that corresponds to the IDS transgene. PCR product depends on the integration of the IDS transgene. In the absence of IDS transgene integration, a second PCR assay detects only mRNA expression corresponding to the albumin mRNA.

FIG. 8 is a set of graphs depicting the levels of plasma IDS in the first six subjects before treatment with the study drug and after treatment over time. Plasma IDS was detectable in all subjects.

FIG. 9 is a set of graphs depicting the levels of GAGs (Heparan sulfate, dermatan sulfate and total glycosaminoglycans), normalized to creatinine, measured in the urine of the first six subjects before treatment with the study drug and after treatment over time.

FIG. 10 is a detailed view of the plasma IDS levels of subject 6 who developed increased alanine aminotransferase (ALT) levels measured in the blood. The dotted line depicts IDS levels in the plasma while the solid line depicts the level of ALT in the blood. Arrows indicate dosing with prednisone.

### DETAILED DESCRIPTION

Disclosed herein are methods and compositions for treating and/or preventing Hunter (MPS II) syndrome in a human subject comprising insertion of a suitable transgene sequence in a target cell. The treatment employs engineered zinc finger nucleases (ZFNs) to site-specifically integrate a corrective copy of the enzyme iduronate-2-sulfatase (hIDS) transgene into the albumin locus of the subject's own hepatocytes in vivo. Once expressed from the integrated transgene, the hIDS is active and able to degrade mucopolysaccharides glycosaminoglycans (GAG). The invention describes methods of prevention or treatment for MPS II subjects.

Normally, IDS enzyme is produced inside the cell and a small amount of it may leak out into the circulation due to cells' imperfect internal transport system. A steady state is established as extracellular enzyme and is taken back up by receptors on the cells' surface. As a result, most of the enzyme normally produced in the body is found in the tissues, and there are generally very small concentrations of enzyme found in circulation. In contrast, ERT is an infusion directly into the bloodstream of a large bolus of enzyme designed to create high concentrations in the circulation to allow uptake into IDS-deficient tissues. However, ERT only produces transient high levels of IDS enzyme, followed by rapid clearance from the circulation within a matter of minutes to hours due to the short half-life of the enzyme, and because large amounts are taken up by the liver. This limits the effectiveness of ERT because it only provides a short window of exposure of enzyme to the tissues, and we know that enzyme uptake by the cells is a slow receptor-mediated process. Instead, an ideal therapy for MPS II would allow prolonged and sustained exposure of the IDS enzyme to the tissues by producing and maintaining continuous, stable levels of enzyme in the circulation. Even low amounts of IDS secreted continuously into the circulation could be adequate to reduce tissue GAGs and potentially provide efficacy for the compositions disclosed herein.

ERT has been shown to increase the amount of lysosomal enzyme activity in patient's leukocytes following treatment, presumably because the cells take up the enzyme from the plasma (leukocytes are lysosome-rich cells). For example, in a study of MPS I patients receiving recombinant IDUA, it was reported (see Kakkis et al. (2001) NEJM 344(3)) that the mean activity of IDUA in leukocytes was 0.04 U per mg prior to treatment, and following treatment, it was measured at 4.98 U per mg seven days after infusion (i.e. immediately prior to the next treatment). Similarly, the measurement of IDS in the circulating leukocytes of MPS II patients can be useful for determining the presence of the enzyme reaching the tissues.

Lysosomal storage diseases (LSDs) are a group of rare metabolic monogenic diseases characterized by the lack of functional individual lysosomal proteins normally involved in the breakdown of waste lipids, mucopolysaccharides (i.e. glycosoaminoglycans (GAG)). These diseases are characterized by a buildup of these compounds in the cell since it is unable to process them for recycling due to the mis-functioning of a specific enzyme in the breakdown pathway. The pathophysiology of LSD was initially thought to be tied to the simple deposition of GAG, but current research has led to an appreciation of the complexities of these diseases. GAG storage appears to lead to the perturbation of cellular, tissue and organ homeostasis, and has also been linked to increased secretion of cytokine and inflammatory modulators leading to an activation of the inflammatory response (Muenzer (2014) Mol Gen Metabol 111:63-72).

Mucopolysaccharidosis II (MPS II), also referred to as Hunter syndrome, is an X-linked, recessive, lysosomal storage disorder found predominantly in males. The incidence of MPS II is reported as 0.3 to 0.71 per 100,000 live births (Burton & Giugliani (2012) Eur J Pediatr. (2012) April; 171(4):631-9). Applying the more conservative median life expectancy of 21.7 years for the attenuated form of the disease (the life expectancy for the severe form of the disease is 11.8 years, (Burrow et al. (2008) Biologics. June; 2(2):311-20; Young & Harper (1982) Med Genet. December; 19(6):408-11) to the yearly incidence yields an estimated prevalence of about 629 individuals with MPS II currently living in the US. MPS II is caused by mutations in the iduronate-2-sulfatase (IDS) gene which encodes an enzyme involved in the lysosomal degradation of the mucopolysaccharides glycosaminoglycans (GAG). This results in the accumulation of GAG in the urine, plasma and tissues and causes multi-systemic, progressive disease. GAGs are the most important biochemical measurement for MPS II. The accumulation of GAGs in cells and tissues, specifically dermatan sulfate and heparan sulfate, is responsible for the underlying pathology and clinical manifestation of MPS II; GAGs were the biochemical marker used by FDA and EMA to assess the pharmacodynamics of intravenous enzyme replacement therapy that is most commonly used to treat MPS II. Hunter syndrome represents a disease spectrum spanning early onset, severe disease (two-thirds of subjects) with somatic and cognitive involvement, to attenuated MPS II characterized by later onset of somatic disease and little or no central nervous system (CNS) disease. The specific type of IDS mutation (>150 gene mutations have been identified) and the levels of the resulting residual IDS enzyme most likely determine the severity of disease. The residual IDS activity in the attenuated form has been measured at 0.2-2.4% of the wildtype IDS activity and those with the severe phenotype have no activity (Sukegawa-Hayasaka et al. (2006) J Inherit Metab Dis 29(6):755-61). The IDS gene is mapped to Xq28, and contains nine exons spread over 24 kb. Major deletions and rearrangements are always associated with the severe form of the disease.

Severe MPS II subjects typically start to have delayed speech and developmental delay between 18 months to 3 years of age. The disease is characterized by symptoms in severe MPS II subjects such as organomegaly, hyperactivity and aggressiveness, neurologic deterioration, joint stiffness and skeletal deformities (including abnormal spinal bones), coarse facial features with enlarged tongue, heart valve thickening, hearing loss and hernias. Joint stiffness leads to problems with walking and manual dexterity. In early childhood, subjects may display an inability to keep up with peers during physical activity, while later in life, the ability to walk even short distances may be lost and many subjects eventually become wheelchair dependent (Raluy-Callado et al. (2013) Orphanet J Rare Dis 8:101). Subjects have frequent upper respiratory infections which initially may be treated by surgical procedures such as adenotonsillectomy but ultimately may require tracheostomy and/or positive pressure ventilation (J. Ed. Wraith (2013) in Emery and Rimoin's Principles and Practice of Medical Genetics, Chapter 102.3, Rimoin, Pyeritz and Korf eds. Elsevier Ltd; Sasaki et al. (1987) Laryngoscope 97: 280-285). Major mortality factors are central nervous system involvement, cardiac involvement, and upper airway obstruction (Sato et al. (2013) Pediatr Cardiol. 34(8):2077-2079). The life expectancy of untreated subjects with severe Hunter syndrome is into the mid teenage years with death due to neurologic deterioration and/or cardiorespiratory failure. Subjects with the attenuated form are typically diagnosed later than the severe subjects. The symptoms of the disease are similar to the severe subjects, but overall disease severity in milder with, in general, slower disease progression with no or only mild cognitive impairment. Death in the untreated attenuated form is often between the ages of 20-30 years from cardiac and respiratory disease.

The only currently approved therapy for MPS II is enzyme replacement therapy (ERT). Intravenous (IV) ERT with recombinant IDS protein (idursulfase; Elaprase®, Shire) has been US FDA approved since 2006 for administration once every week in a dose of 0.5 mg/kg of body weight and has been shown to improve walking capacity in MPS II subjects 5 years and older. Limitations to ERT include the need for life-long treatment, development of neutralizing antibodies, inability of the enzyme to cross the blood brain barrier, and the inconvenience of weekly intravenous infusions. In addition, Elaprase® has a very short half-life in the plasma following treatment. When given at the approved dose (0.5 mg/kg administered weekly as a 3-hour infusion), the protein has an approximate half-life of 44 minutes (Elaprase® Solution for Intravenous Infusion Prescribing Information, Shire Human Genetics Therapies, Cambridge Mass. 2007 October). Because idursulfase cannot cross into the CNS, ERT has little to no impact on cognitive function (Parini et al. (2015) Mol Gen Metabol Rep 3:65-74). It has also been suggested to have limited efficacy for the treatment of cardiac valve disease associated with MPS II (Sato et al., ibid). In contrast to Hurler syndrome (the severe form of MPS I), hematopoietic stem cell transplantation (HSCT) has not historically been recommended for the severe form of MPS II due to a lack of efficacy in treating cognitive impairment (Guffon et al. (2009) J. Pediatric 154(5):733).

In preliminary analysis, molecular and enzymatic evidence has been found by editing of the human genome in vivo (inside the body). The compositions disclosed herein are meant to treat a subject's liver such that a continuous supply of functional IDS enzyme is produced for the duration of the subject's life. The preliminary evidence of in vivo genome editing suggests that genome-edited liver cells may be able to generate IDS enzyme in patients with MPS II, however more data are needed to understand whether the IDS enzyme increase will improve clinical outcomes. The interim results suggest that in vivo editing has occurred in the liver and the biochemical data (for example, the production and secretion of active IDS enzyme into the blood) suggest that genome-edited liver cells are able to generate IDS enzyme in patients with MPS II. Liver biopsies have so far been analyzed for three eligible subjects—one from the low-dose cohort and two from the mid-dose cohort. In liver tissue samples from both mid-dose cohort subjects, a molecular signal of successful genome editing was detected using a gene integration assay. This test uses a reverse transcriptase polymerase chain reaction (RT-PCR) method to identify albumin-IDS chimeric mRNA transcripts. The detection of albumin-IDS mRNA fusion transcript in the biopsy samples from the two subjects in the mid-dose cohort indicates ZFN-mediated integration of the IDS gene has occurred at the expected site within the endogenous albumin gene. A separate liver tissue analysis conducted using MiSeq DNA sequencing, a less sensitive assay (lower limit of quantification of 0.1%), did not detect editing in samples from the low and mid-dose cohort subjects. Second-generation, potentially more potent ZFN constructs (for example, SB-71557 and SB-71728) were designed to increase editing efficiency, among other improvements. The preclinical data showed three potential ZFN 2.0 advantages: (1) a 5- to 30-fold improvement in efficiency and potency due to structural changes; (2) the ability to function equally well in the patients who have a single nucleotide polymorphism (SNP) in the target locus in the albumin gene (approximately 20% of the population); and, (3) improved specificity (see U.S. application Ser. No. 16/271,250). These ZFN compositions will also be tested.

A newly developed sensitive quantitative assay (lower limit of quantification of 0.78 nmol/hour/mL) was used to measure plasma IDS activity. Minor increases in IDS enzyme activity compared to baseline were recorded in the two subjects receiving the mid-dose and in one subject receiving the high dose, yet at 24 weeks these measurements remained within the expected range for baseline values (less than 10 nmol/hour/mL, as compared to the normal range which is estimated at greater than 82 nmol/hour/mL).

A substantial increase in plasma IDS activity was measured in the second patient in the high dose cohort, with levels rising to approximately 50 nmol/hour/mL by week 6 following administration of a composition as described herein. The plasma IDS activity levels subsequently decreased concurrently with development of a mild transaminitis, which is a known risk of AAV-based therapies due to a suspected immune response. Grade 1 elevations in liver function tests were measured at Day 62, 111 and 128. The patient was hospitalized on Day 121 for an incarcerated umbilical hernia unrelated to study drug. As of the most recent observation, the patient's plasma IDS activity measured 14 nmol/hour/mL, which is above the baseline value but below the normal range.

Baseline urine GAG measurements for all six patients were in a range considered at or slightly above normal, except for heparan sulfate which was elevated in all subjects at baseline. The overall results did not show substantial increases or decreases in GAGs in relation to the dose of the compositions disclosed herein, after accounting for baseline variability.

The clinical relevance of the biochemical changes observed after administration of the compositions disclosed herein will be assessed as clinical data and patient outcomes are analyzed following a trial of withdrawal from ERT. To date, two mid-dose and one high-dose patients have initiated ERT withdrawal. One mid-dose patient who initiated the procedure resumed ERT approximately 3 months later due to fatigue and increasing GAGs. Additional data readouts in the future will include biochemical data from the high-dose cohort expansion subjects, liver biopsy analysis, and outcomes following ERT withdrawal.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. The term “zinc finger nuclease” includes one ZFN as well as a pair of ZFNs (the members of the pair are referred to as “left and right” or “first and second” or “pair”) that dimerize to cleave the target gene.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. Nos. 8,586,526 and 9,458,205. The term “TALEN” includes one TALEN as well as a pair of TALENs (the members of the pair are referred to as “left and right” or “first and second” or “pair”) that dimerize to cleave the target gene. Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 8,568,526; 6,140,081; 6,453,242; and 6,534,261; see also International Patent Publication Nos. WO 98/53058; WO 98/53059; WO 98/53060; WO 02/016536; and WO 03/016496.

A “selected” zinc finger protein or TALE is a protein not found in nature whose production results primarily from an empirical process such as phage display, interaction trap or hybrid selection. See e.g., U.S. Pat. Nos. 8,586,526; 5,789,538; 5,925,523; 6,007,988; 6,013,453; 6,200,759; and International Patent Publication Nos. WO 95/19431; WO 96/06166; WO 98/53057; WO 98/54311; WO 00/27878; WO 01/60970; WO 01/88197; and WO 02/099084.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger or TALEN proteins can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or non-coding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, U.S. Pat. Nos. 7,888,121; 7,914,796; 8,034,598 and 8,823,618, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “disease associated gene” is one that is defective in some manner in a monogenic disease. Non-limiting examples of monogenic diseases include severe combined immunodeficiency, cystic fibrosis, lysosomal storage diseases (e.g. Gaucher's, Hurler's Hunter's, Fabry's, Neimann-Pick, Tay-Sach's etc), sickle cell anemia, and thalassemia.

The “blood brain barrier” is a highly selective permeability barrier that separates the circulating blood from the brain in the central nervous system. The blood brain barrier is formed by brain endothelial cells which are connected by tight junctions in the CNS vessels that restrict the passage of blood solutes. The blood brain barrier has long been thought to prevent the uptake of large molecule therapeutics and prevent the uptake of most small molecule therapeutics (Pardridge (2005) NeuroRx 2(1):3-14).

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of fusion molecules include, but are not limited to, fusion proteins (for example, a fusion between a protein DNA-binding domain and a cleavage domain), fusions between a polynucleotide DNA-binding domain (e.g., sgRNA) operatively associated with a cleavage domain, and fusion nucleic acids (for example, a nucleic acid encoding the fusion protein).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALEN as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

“Red Blood Cells” (RBCs) or erythrocytes are terminally differentiated cells derived from hematopoietic stem cells. They lack a nuclease and most cellular organelles. RBCs contain hemoglobin to carry oxygen from the lungs to the peripheral tissues. In fact, 33% of an individual RBC is hemoglobin. They also carry CO2 produced by cells during metabolism out of the tissues and back to the lungs for release during exhale. RBCs are produced in the bone marrow in response to blood hypoxia which is mediated by release of erythropoietin (EPO) by the kidney. EPO causes an increase in the number of proerythroblasts and shortens the time required for full RBC maturation. After approximately 120 days, since the RBC do not contain a nucleus or any other regenerative capabilities, the cells are removed from circulation by either the phagocytic activities of macrophages in the liver, spleen and lymph nodes (˜90%) or by hemolysis in the plasma (˜10%). Following macrophage engulfment, chemical components of the RBC are broken down within vacuoles of the macrophages due to the action of lysosomal enzymes.

“Secretory tissues” are those tissues in an animal that secrete products out of the individual cell into a lumen of some type which are typically derived from epithelium. Examples of secretory tissues that are localized to the gastrointestinal tract include the cells that line the gut, the pancreas, and the gallbladder. Other secretory tissues include the liver, tissues associated with the eye and mucous membranes such as salivary glands, mammary glands, the prostate gland, the pituitary gland and other members of the endocrine system. Additionally, secretory tissues include individual cells of a tissue type which are capable of secretion.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to up-regulate gene expression. When a fusion polypeptide in which a ZFP or TALE DNA-binding domain is fused to a cleavage domain, the ZFP or TALE DNA-binding domain and the cleavage domain are in operative linkage if, in the fusion polypeptide, the ZFP or TALE DNA-binding domain portion is able to bind its target site and/or its binding site, while the cleavage domain is able to cleave DNA in the vicinity of the target site.

A “functional” protein, polypeptide or nucleic acid includes any protein, polypeptide or nucleic acid that provides the same function as the wild-type protein, polypeptide or nucleic acid. A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and International Patent Publication No. WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest. A “WPRE” sequence is a woodchuck hepatitis posttranscriptional regulatory element derived from the woodchuck hepatitis virus. WPRE is a 600 bp long tripartite element containing gamma, alpha, and beta elements, in the given order (Donello et al. (1992) J Virol 72:5085-5092) and contributes to the strong expression of transgenes in AAV systems (Loeb et al. (1999) Hum Gene Ther 10:2295-2305). It also enhances the expression of a transgene lacking introns. In its natural form WPRE contains a partial open reading frame (ORF) for the WHV-X protein. The fully expressed WHV-X protein in the context of other viral elements like the WHV (We2) enhancer has been associated with a higher risk of hepatocarcinoma in woodchucks and mice (Hohne et. al (1990) EMBO J 9(4):1137-45; Flajolet et. al (1998) J Virol 72(7):6175-80). The WHV-X protein does not appear to be directly oncogenic, but some studies suggest that under certain circumstances it can act as a weak cofactor for the generation of liver cancers associated with infection by hepadnaviruses (hepatitis B virus for man; woodchuck hepatitis virus for woodchucks). Many times, mention of “wildtype” WPRE is referring to a 591 bp sequence (nucleotides 1094-1684 in GenBank accession number J02442) containing a portion of the WHV X protein open-reading frame (ORF) in its 3′ region. In this element, there is an initial ATG start codon for WHV-X at position 1502 and a promoter region with the sequence GCTGA at position 1488. In Zanta-Boussif (ibid), a mut6WPRE sequence was disclosed wherein the promoter sequence at position 1488 was modified to ATCAT and the start codon at position 1502 was modified to TTG, effectively prohibiting expression of WHV-X. In the J04514.1 WPRE variant, the ATG WHV X start site is a position 1504, and a mut6 type variant can be made in the this J04514.1 strain. Another WPRE variant is the 247 bp WPRE3 variant comprising only minimal gamma and alpha elements from the wild type WPRE (Choi et al. (2014) Mol Brain 7:17), which lacks the WHV X sequences.

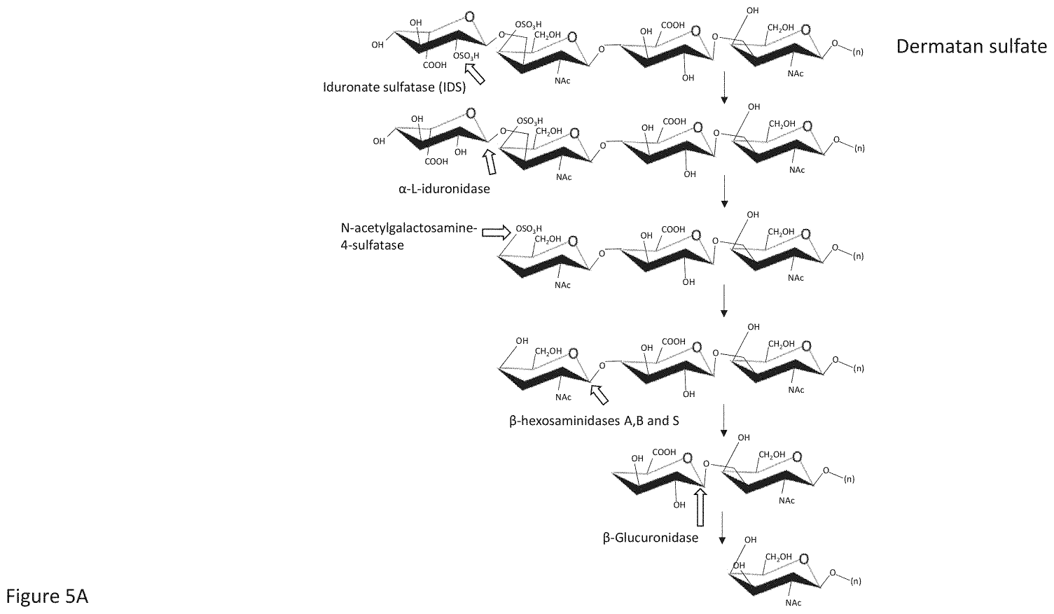

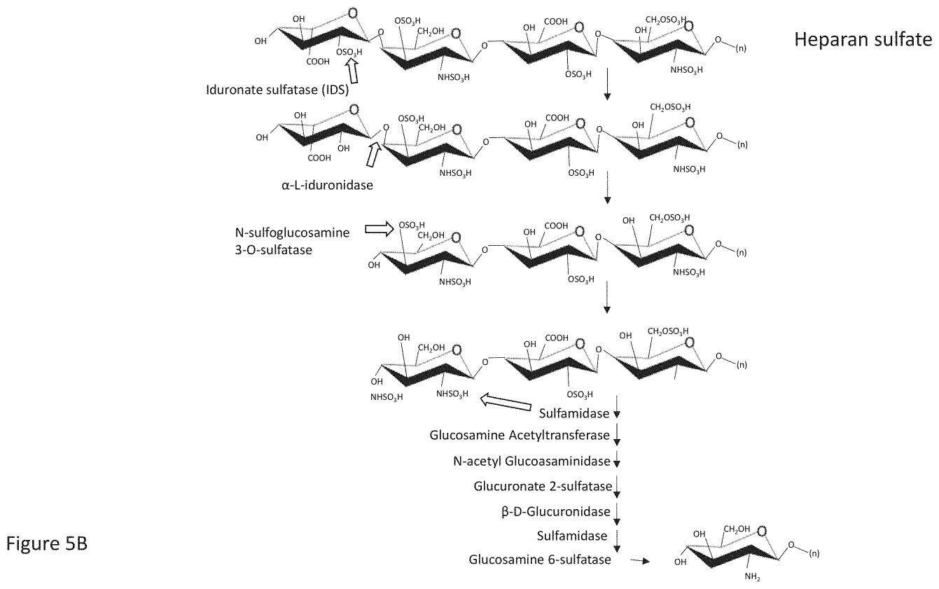

The extracellular matrix that surrounds and binds certain types of cells is composed of numerous components, including fibrous structural proteins, such as various collagens, adhesive proteins like laminin and fibronectin, and proteoglycans that form the gel into which the fibrous structural proteins are embedded. Proteoglycans are very large macromolecules consisting of a core protein to which many long polysaccharide chains called glycosaminoglycans are covalently bound. Due to the high negative charge of the glycosaminoglycans, the proteoglycans are very highly hydrated, a property that allows the proteoglycans to form a gel-like matrix that can expand and contract. The proteoglycans are also effective lubricants. “Glycosoaminoglycans” or “GAGs” are long, linear polymers of unbranched polysaccharides consisting of a repeating disaccharide unit. The repeating unit (except for keratan) consists of an amino hexose sugar (N-acetylglucosamine or N-acetylgalactosamine) along with an acidic uronic sugar (glucuronic acid or iduronic acid) or galactose. The exception to this general structure is keratan sulfate, which has galactose in place of the acidic hexose. Glycosaminoglycans are highly polar and attract water. All of the GAGs except hyaluronan are covalently linked to one of approximately 30 different core proteins to form proteoglycans. The core protein is synthesized on the rough endoplasmic reticulum and transferred to the Golgi where nucleoside diphosphate-activated acidic and amino sugars are alternately added to the nonreducing end of the growing polysaccharide by glycosyltransferases, resulting in the characteristic repeating disaccharide structure common to the GAGs. Heparin/heparan sulfate (HS GAGs) and chondroitin sulfate/dermatan sulfate (CS GAGs) are synthesized in the Golgi apparatus, where protein cores made in the rough endoplasmic reticulum are posttranslationally modified with O-linked glycosylations by glycosyltransferases forming proteoglycans. Keratan sulfate may modify core proteins through N-linked glycosylation or O-linked glycosylation of the proteoglycan. The fourth class of GAG, hyaluronic acid, is not synthesized by the Golgi, but rather by integral membrane synthases which immediately secrete the dynamically elongated disaccharide chain. Degradation of proteoglycans during normal turnover of the extracellular matrix begins with proteolytic cleavage of the core protein by proteases in the extracellular matrix, which then enters the cell via endocytosis. The endosomes deliver their content to the lysosomes, where the proteolytic enzymes complete the degradation of the core proteins and an array of glycosidases and sulfatases hydrolyze the GAGs to monosaccharides. The lysosomes contain both endoglycosidases, which hydrolyze the long polymers into shorter oligosaccharides, and exoglycosidases that cleave individual acidic- or aminosugars from the GAG fragments. Lysosomal catabolism of GAGs proceeds in a stepwise manner from the non-reducing end (see FIG. 5). If the terminal sugar is sulfated, then the sulfate bond must be hydrolyzed by a specific sulfatase before the sugar can be removed. When the sulfate has been removed, a specific exoglycosidase then hydrolyzes the terminal sugar from the nonreducing end of the oligosaccharide, thus leaving it 1 sugar shorter. Degradation continues in this stepwise fashion, alternating between removal of sulfates by sulfatases and cleavage of the terminal sugars by exoglycosidases. If removal of a sulfate leaves a terminal glucosamine residue, then it must first be acetylated to N-acetylglucosamine because the lysosome lacks the enzyme required to remove glucosamine. This is accomplished by an acetyltransferase that uses acetyl-CoA as the acetyl group donor. When the glucosamine residue has been N-acetylated it can be hydrolyzed by α-N-acetylglucosaminidase, allowing the continuation of the stepwise degradation of the GAG. In the case of MPS II, the terminal sugar of heparan sulfate and dermatan sulfate are sulfated, and the defective IDS enzyme is not able to remove that sulfate group. Normally, the sulfate on the terminal sugar group would be removed by iduronate sulfatase (IDS) and then the GAG would be acted on by alpha iduromidase (IDUA) for removal of the terminal sugar.

The terms “subject” and “patient” are used interchangeably and refer to mammals such as human subjects and non-human primates, as well as experimental animals such as rabbits, dogs, cats, rats, mice, and other animals. Accordingly, the term “subject” or “patient” as used herein means any mammalian subject to which the altered cells of the invention and/or proteins produced by the altered cells of the invention can be administered. Subjects of the present invention include those having MPS II disorder.

Generally, the subject is eligible for treatment for MPS II. For the purposes herein, such eligible subject is one who is experiencing, has experienced, or is likely to experience, one or more signs, symptoms or other indicators of MPS II; has been diagnosed with MPS II, whether, for example, newly diagnosed, and/or is at risk for developing MPS II. One suffering from or at risk for suffering from MPS II may optionally be identified as one who has been screened for elevated levels of GAG in tissues and/or urine.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life.

As used herein, “delaying” or “slowing” the progression of MPS II means to prevent, defer, hinder, slow, retard, stabilize, and/or postpone development of the disease. This delay can be of varying lengths of time, depending on the history of the disease and/or individual being treated.

An “effective dose” or “effective amount” is a dose and/or amount of the composition given to a subject as disclosed herein effective to stabilize, decrease or eliminate urine GAG and/or result in measurable IDS activity in the plasma.

As used herein, “at the time of starting treatment” refers to the time period at or prior to the first exposure to a MPS II therapeutic composition such as the compositions of the invention. In some embodiments, “at the time of starting treatment” is about any of one year, nine months, six months, three months, second months, or one month prior to a MPS II drug, such as SB-913. In some embodiments, “at the time of starting treatment” is immediately prior to coincidental with the first exposure to a MPS II therapeutic composition.

The term “wheelchair dependent” means a subject that is unable to walk through injury or illness and must rely on a wheelchair to move around.

The term “mechanical ventilator” describes a device that improves the exchange of air between a subject's lungs and the atmosphere.

As used herein, “based upon” includes (1) assessing, determining, or measuring the subject characteristics as described herein (and preferably selecting a subject suitable for receiving treatment; and (2) administering the treatment(s) as described herein.

A “symptom” of MPS II is any phenomenon or departure from the normal in structure, function, or sensation, experienced by the subject and indicative of MPS II.

“Severe MPS II” in subjects is characterized by delayed speech and developmental delay between 18 months to 3 years of age. The disease is characterized in severe MPS II subjects by organomegaly, hyperactivity and aggressiveness, neurologic deterioration, joint stiffness and skeletal deformities (including abnormal spinal bones), coarse facial features with enlarged tongue, heart valve thickening, hearing loss and hernias. The life expectancy of untreated subjects with severe Hunter syndrome is into the mid teenage years with death due to neurologic deterioration and/or cardiorespiratory failure.

“Attenuated form MPS II” in subjects are typically diagnosed later than the severe subjects. The somatic clinical features are similar to the severe subjects, but overall disease severity in milder with, in general, slower disease progression with no or only mild cognitive impairment. Death in the untreated attenuated form is often between the ages of 20-30 years from cardiac and respiratory disease.

The term “supportive surgery” refers to surgical procedures that may be performed on a subject to alleviate symptoms that may be associated with a disease. For subjects with MPS II, such supportive surgeries may include heart valve replacement surgery, tonsillectomy and adenoidectomy, placement of ventilating tubes, repair of abdominal hernias, cervical decompression, treatment of carpal tunnel syndrome, surgical decompression of the median nerve, instrumented fusion (to stabilize and strengthen the spine), arthroscopy, hip or knee replacement, and correction of the lower limb axis, and tracheostomy (see Wraith et al. (2008) Eur J Pediatr. 167(3):267-277; and Scarpa et al. (2011) Orphanet Journal of Rare Diseases 6:72).

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal anti-inflammatory drugs (NSAIDs); ganciclovir, tacrolimus, glucocorticoids such as Cortisol or aldosterone, antiinflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antagonists including anti-interferon-alpha, -beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (International Patent Publication No. WO 90/08187 published Jul. 26, 1990); streptokinase; TGF-beta; streptodornase; RNA or DNA from the host; FK506; RS-61443; deoxysperguahn; rapamycin; T-cell receptor (Cohen et al., U.S. Pat. No. 5,114,721); T-cell receptor fragments (Offner et al. (1991) Science 251:430-432; International Patent Publication No. WO 90/11294; Janeway (1989) Nature 341:482; and International Patent Publication No. WO 91/01133); and T cell receptor antibodies (EP 340,109) such as T10B9.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone, glucocorticoid and betamethasone.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

A “label” is used herein to refer to information customarily included with commercial packages of pharmaceutical formulations including containers such as vials and package inserts, as well as other types of packaging.

It is to be understood that one, some, or all of the properties of the various embodiments described herein may be combined to form other embodiments of the present invention. These and other aspects of the invention will become apparent to one of skill in the art.

Nucleases

The methods described herein can make use of one or more nucleases for targeted introduction of the IDS transgene. Non-limiting examples of nucleases include ZFNs, TALENs, homing endonucleases, CRISPR/Cas and/or Ttago guide RNAs, that are useful for in vivo cleavage of a donor molecule carrying a transgene and nucleases for cleavage of the genome of a cell such that the transgene is integrated into the genome in a targeted manner. In certain embodiments, one or more of the nucleases are naturally occurring. In other embodiments, one or more of the nucleases are non-naturally occurring, i.e., engineered in the DNA-binding molecule (also referred to as a DNA-binding domain) and/or cleavage domain. For example, the DNA-binding domain of a naturally-occurring nuclease may be altered to bind to a selected target site (e.g., a ZFP, TALE and/or sgRNA of CRISPR/Cas that is engineered to bind to a selected target site). In other embodiments, the nuclease comprises heterologous DNA-binding and cleavage domains (e.g., zinc finger nucleases; TAL-effector domain DNA binding proteins; meganuclease DNA-binding domains with heterologous cleavage domains). In other embodiments, the nuclease comprises a system such as the CRISPR/Cas of Ttago system.

A. DNA-Binding Domains

In certain embodiments, the composition and methods described herein employ a meganuclease (homing endonuclease) DNA-binding domain for binding to the donor molecule and/or binding to the region of interest in the genome of the cell. Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22:1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

In certain embodiments, the methods and compositions described herein make use of a nuclease that comprises an engineered (non-naturally occurring) homing endonuclease (meganuclease). The recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22:1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 2007/0117128. The DNA-binding domains of the homing endonucleases and meganucleases may be altered in the context of the nuclease as a whole (i.e., such that the nuclease includes the cognate cleavage domain) or may be fused to a heterologous cleavage domain.

In other embodiments, the DNA-binding domain of one or more of the nucleases used in the methods and compositions described herein comprises a naturally occurring or engineered (non-naturally occurring) TAL effector DNA binding domain. See, e.g., U.S. Pat. No. 8,586,526, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like (TAL) effectors which mimic plant transcriptional activators and manipulate the plant transcriptome (see Kay et al. (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al. (1989) Mol Gen Genet 218:127-136 and International Patent Publication No. WO 2010/079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al. (2006) J Plant Physiol 163(3):256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al. (2007) Appl and Envir Micro 73(13):4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas. See, e.g., U.S. Pat. No. 8,586,526, incorporated by reference in its entirety herein.

Specificity of these TAL effectors depends on the sequences found in the tandem repeats. The repeated sequence comprises approximately 102 bp and the repeats are typically 91-100% homologous with each other (Bonas et al., ibid). Polymorphism of the repeats is usually located at positions 12 and 13 and there appears to be a one-to-one correspondence between the identity of the hypervariable diresidues (RVDs) at positions 12 and 13 with the identity of the contiguous nucleotides in the TAL-effector's target sequence (see Moscou and Bogdanove (2009) Science 326:1501 and Boch et al. (2009) Science 326:1509-1512). Experimentally, the natural code for DNA recognition of these TAL-effectors has been determined such that an HD sequence at positions 12 and 13 leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al., ibid). Engineered TAL proteins have been linked to a FokI cleavage half domain to yield a TAL effector domain nuclease fusion (TALEN) exhibiting activity in a yeast reporter assay (plasmid based target). See, e.g., U.S. Pat. No. 8,586,526; Christian et al. (2010) Genetics epub 10.1534/genetics.110.120717).

In certain embodiments, the DNA binding domain of one or more of the nucleases used for in vivo cleavage and/or targeted cleavage of the genome of a cell comprises a zinc finger protein. Preferably, the zinc finger protein is non-naturally occurring in that it is engineered to bind to a target site of choice. See, for example, See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; and 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197. In addition, enhancement of binding specificity for zinc finger binding domains has been described, for example, in co-owned International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 8,772,453; 6,479,626; 6,903,185; and 7,153,949 for exemplary linker sequences. The proteins described herein may include any combination of suitable linkers between the individual zinc fingers of the protein.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; and 6,200,759; and International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

In certain embodiments, the DNA-binding domain of the nuclease is part of a CRISPR/Cas nuclease system, including, for example a single guide RNA (sgRNA). See, e.g., U.S. Pat. No. 8,697,359 and U.S. Patent Publication No. 2015/0056705. The CRISPR (clustered regularly interspaced short palindromic repeats) locus, which encodes RNA components of the system, and the Cas (CRISPR-associated) locus, which encodes proteins (Jansen et al. (2002) Mol. Microbiol. 43:1565-1575; Makarova et al. (2002) Nucleic Acids Res. 30:482-496; Makarova et al. (2006) Biol. Direct 1:7; Haft et al. (2005) PLoS Comput. Biol. 1:e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein. Additional non-limiting examples of RNA guided nucleases that may be used in addition to and/or instead of Cas proteins include Class 2 CRISPR proteins such as Cpf1. See, e.g., Zetsche et al. (2015) Cell 163:1-13.

In some embodiments, the DNA binding domain is part of a TtAgo system (see Swarts et al. (2014) Nature 507(7491):258-261; Swarts et al. (2012) PLoS One 7(4):e35888; Sheng et al. (2014) Proc. Natl. Acad. Sci. U.S.A. 111(2):652-657). In eukaryotes, gene silencing is mediated by the Argonaute (Ago) family of proteins. In this paradigm, Ago is bound to small (19-31 nt) RNAs. This protein-RNA silencing complex recognizes target RNAs via Watson-Crick base pairing between the small RNA and the target and endonucleolytically cleaves the target RNA (Vogel (2014) Science 344:972-973). In contrast, prokaryotic Ago proteins bind to small single-stranded DNA fragments and likely function to detect and remove foreign (often viral) DNA (Yuan et al. (2005) Mol. Cell 19:405; Olovnikov et al. (2013) Mol. Cell 51:594; Swarts et al., ibid). Exemplary prokaryotic Ago proteins include those from Aquifex aeolicus, Rhodobacter sphaeroides, and Thermus thermophilus.

One of the most well-characterized prokaryotic Ago protein is the one from T. thermophilus (TtAgo; Swarts et al., ibid). TtAgo associates with either 15 nt or 13-25 nt single-stranded DNA fragments with 5′ phosphate groups. This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (G. Sheng et al., ibid). Ago from Rhodobacter sphaeroides (RsAgo) has similar properties (Olovnikov et al., ibid).

Exogenous guide DNAs of arbitrary DNA sequence can be loaded onto the TtAgo protein (Swarts et al., ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Further, it might be preferable to use a version of the TtAgo protein that has been altered via mutagenesis to have improved activity at 37 degrees Celsius. TtAgo-RNA-mediated DNA cleavage could be used to effect a panopoly of outcomes including gene knock-out, targeted gene addition, gene correction, targeted gene deletion using techniques standard in the art for exploitation of DNA breaks.

Thus, the nuclease comprises a DNA-binding domain in that specifically binds to a target site in any gene into which it is desired to insert a donor (transgene).

In certain embodiments the DNA-binding domains bind to albumin, e.g., DNA-binding domains of the ZFPs designated SBS-47171 and SBS-47898. See, e.g., U.S. Patent Publication No. 2015/0159172.

B. Cleavage Domains

Any suitable cleavage domain can be associated with (e.g., operatively linked) to a DNA-binding domain to form a nuclease. For example, ZFP DNA-binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity. See, e.g., Kim et al. (1996) Proc Natl Acad Sci USA 93(3):1156-1160. More recently, ZFNs have been used for genome modification in a variety of organisms. See, for example, U.S. Patent Publication Nos. 2003/0232410; 2005/0208489; 2005/0026157; 2005/0064474; 2006/0188987; 2006/0063231; and International Patent Publication No. WO 07/014275. Likewise, TALE DNA-binding domains have been fused to nuclease domains to create TALENs. See, e.g., U.S. Pat. No. 8,586,526. CRISPR/Cas nuclease systems comprising single guide RNAs (sgRNAs) that bind to DNA and associate with cleavage domains (e.g., Cas domains) to induce targeted cleavage have also been described. See, e.g., U.S. Pat. Nos. 8,697,359 and 8,932,814 and U.S. Patent Publication No. 2015/0056705.

As noted above, the cleavage domain may be heterologous to the DNA-binding domain, for example a zinc finger DNA-binding domain and a cleavage domain from a nuclease or a TALEN DNA-binding domain and a cleavage domain from a nuclease; a sgRNA DNA-binding domain and a cleavage domain from a nuclease (CRISPR/Cas); and/or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However, any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150; and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95:10,570-10,575. Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using zinc finger-FokI fusions, two fusion proteins, each comprising a FokI cleavage half-domain, can be used to reconstitute a catalytically active cleavage domain. Alternatively, a single polypeptide molecule containing a zinc finger binding domain and two FokI cleavage half-domains can also be used. Parameters for targeted cleavage and targeted sequence alteration using zinc finger-FokI fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in U.S. Pat. No. 7,888,121, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Pat. Nos. 8,772,453; 8,623,618; 8,409,861; 8,034,598; 7,914,796; and 7,888,121, the disclosures of all of which are incorporated by reference in their entireties herein. Amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains.

Exemplary engineered cleavage half-domains of FokI that form obligate heterodimers include a pair in which a first cleavage half-domain includes mutations at amino acid residues at positions 490 and 538 of FokI and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. U.S. Pat. Nos. 7,914,796 and 8,034,598, the disclosures of which are incorporated by reference in their entireties. In certain embodiments, the engineered cleavage half-domain comprises mutations at positions 486, 499 and 496 (numbered relative to wild-type FokI), for instance mutations that replace the wild type Gln (Q) residue at position 486 with a Glu(E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). See, e.g., U.S. Pat. No. 8,772,453. In other embodiments, the engineered cleavage half domain comprises the “Sharkey” and/or “Sharkey mutations” (see Guo et al. (2010) J. Mol. Biol. 400(1):96-107).

Engineered cleavage half-domains described herein can be prepared using any suitable method, for example, by site-directed mutagenesis of wild-type cleavage half-domains (FokI) as described in U.S. Pat. Nos. 7,888,121; 7,914,796; 8,034,598; and 8,623,618.

Alternatively, nucleases may be assembled in vivo at the nucleic acid target site using so-called “split-enzyme” technology (see, e.g. U.S. Patent Publication No. 2009/0068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in U.S. Pat. No. 8,563,314. Expression of the nuclease may be under the control of a constitutive promoter or an inducible promoter, for example the galactokinase promoter which is activated (de-repressed) in the presence of raffinose and/or galactose and repressed in presence of glucose.

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek et al. (2013) Elife 2:e00471. doi: 10.7554/eLife.00471.; Jinek et al. (2012) Science 337:816-821; and Cong, ibid).

The nuclease(s) as described herein may make one or more double-stranded and/or single-stranded cuts in the target site. In certain embodiments, the nuclease comprises a catalytically inactive cleavage domain (e.g., FokI and/or Cas protein). See, e.g., U.S. Pat. Nos. 9,200,266 and 8,703,489 and Guillinger et al. (2014) Nature Biotech. 32(6):577-582. The catalytically inactive cleavage domain may, in combination with a catalytically active domain act as a nickase to make a single-stranded cut. Therefore, two nickases can be used in combination to make a double-stranded cut in a specific region. Additional nickases are also known in the art, for example, McCaffery et al. (2016) Nucleic Acids Res. 44(2):e11. doi: 10.1093/nar/gkv878. Epub 2015 Oct. 19.

Thus, any nuclease comprising a DNA-binding domain and cleavage domain can be used. In certain embodiments, the nuclease comprises a ZFN made up of left and right ZFNs, for example a ZFN comprising a first ZFN comprising a ZFP designated SBS-47171 and a cleavage domain and a second ZFN comprising a ZFP designated SBS-47898 and a cleavage domain. In certain embodiments, the left and right (first and second) ZFNs of the ZFN are carried on the same vector and in other embodiments, the paired components of the ZFN are carried on different vectors, for example two AAV vectors, one designated SB-47171 AAV as shown in Table 1, SEQ ID NO:9 (an AAV2/6 vector carrying ZFN comprising the ZFP designated SBS-47171) and the other designated SB-47898 AAV as shown in Table 2, SEQ ID NO:12 (an AAV2/6 vector carrying ZFN comprising the ZFP designated SB S-47898).

Target Sites

As described in detail above, DNA domains can be engineered to bind to any sequence of choice in a locus, for example an albumin or other safe-harbor gene. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, co-owned U.S. Pat. Nos. 6,453,242 and 6,534,261, incorporated by reference herein in their entireties. Rational design of TAL-effector domains can also be performed. See, e.g., U.S. Patent Publication No. 2011/0301073.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237.

Selection of target sites; nucleases and methods for design and construction of fusion proteins (and polynucleotides encoding same) are known to those of skill in the art and described in detail in U.S. Patent Publication Nos. 2005/0064474 and 2006/0188987, incorporated by reference in their entireties herein.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Pat. No. 8,586,526.

In certain embodiments, the target site(s) for the DNA-binding domain(s) (is)are within an albumin gene. See, e.g., U.S. Patent Publication No. 2015/0159172.

Donors

As noted above, insertion of an exogenous sequence (also called a “donor sequence” or “donor”), for example for correction of a mutant gene or for increased expression of a gene encoding a protein lacking or deficient in MPS II disease (e.g., IDS) is provided. In some embodiments, transgene function can be measured by a stabilization, decline or elimination of GAG in the urine and/or by measuring transgene expression (for example, hIDS) and/or activity in the plasma of a subject treated with the methods and compositions disclosed herein. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

Described herein are methods of targeted insertion of a transgene encoding an IDS protein for insertion into a chosen location. Polynucleotides for insertion can also be referred to as “exogenous” polynucleotides, “donor” polynucleotides or molecules or “transgenes.” The donor polynucleotide can be DNA or RNA, single-stranded and/or double-stranded and can be introduced into a cell in linear or circular form. See, e.g., U.S. Pat. Nos. 8,703,489 and 9,005,973. The donor sequence(s) can also be contained within a DNA MC, which may be introduced into the cell in circular or linear form. See, e.g., U.S. Patent Publication No. 2014/0335063. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted (e.g., highly expressed, albumin, AAVS1, HPRT, etc.). However, it will be apparent that the donor may comprise a promoter and/or enhancer, for example a constitutive promoter or an inducible or tissue specific promoter. In some embodiments, the donor is maintained in the cell in an expression plasmid such that the gene is expressed extra-chromosomally.

The donor molecule may be inserted into an endogenous gene such that all, some or none of the endogenous gene is expressed. For example, a transgene as described herein may be inserted into an albumin or other locus such that some (N-terminal and/or C-terminal to the transgene encoding the lysosomal enzyme) or none of the endogenous albumin sequences are expressed, for example as a fusion with the transgene encoding the IDS protein(s). In other embodiments, the transgene (e.g., with or without additional coding sequences such as for albumin) is integrated into any endogenous locus, for example a safe-harbor locus.

When endogenous sequences (endogenous or part of the transgene) are expressed with the transgene, the endogenous sequences (e.g., albumin, etc.) may be full-length sequences (wild-type or mutant) or partial sequences. Preferably the endogenous sequences are functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the exogenous sequence (donor) comprises a fusion of a protein of interest and, as its fusion partner, an extracellular domain of a membrane protein, causing the fusion protein to be located on the surface of the cell. This allows the protein encoded by the transgene to potentially act in the serum. In the case of treatment for MPS II disease, IDS enzyme encoded by the transgene fusion acts on the metabolic products that are accumulating in the serum from its location on the surface of the cell (e.g., RBC). In addition, if the RBC is engulfed by a splenic macrophage as is the normal course of degradation, the lysosome formed when the macrophage engulfs the cell would expose the membrane bound fusion protein to the high concentrations of metabolic products in the lysosome at the pH more naturally favorable to that enzyme.

In some cases, the donor may be an endogenous gene (IDS) that has been modified. For instance, codon optimization may be performed on the endogenous gene to produce a donor. Furthermore, although antibody response to enzyme replacement therapy varies with respect to the specific therapeutic enzyme in question and with the individual subject, a significant immune response has been seen in many MPS II disease subjects being treated with enzyme replacement with wild-type IDS. In addition, the relevance of these antibodies to the efficacy of treatment is also variable (see Katherine Ponder (2008) J Clin Invest 118(8):2686). Thus, the methods and compositions of the current invention can comprise the generation of donor with modified sequences as compared to wild-type IDS, including, but not limited to, modifications that produce functionally silent amino acid changes at sites known to be priming epitopes for endogenous immune responses, and/or truncations such that the polypeptide produced by such a donor is less immunogenic.

MPS II disease subjects often have neurological sequelae due the lack of the missing IDS enzyme in the brain. Unfortunately, it is often difficult to deliver therapeutics to the brain via the blood due to the impermeability of the blood brain barrier. Thus, the methods and compositions of the invention may be used in conjunction with methods to increase the delivery of the therapeutic into the brain, including but not limited to methods that cause a transient opening of the tight junctions between cells of the brain capillaries such as transient osmotic disruption through the use of an intracarotid administration of a hypertonic mannitol solution, the use of focused ultrasound and the administration of a bradykinin analogue. Alternatively, therapeutics can be designed to utilize receptors or transport mechanisms for specific transport into the brain. Examples of specific receptors that may be used include the transferrin receptor, the insulin receptor or the low-density lipoprotein receptor related proteins 1 and 2 (LRP-1 and LRP-2). LRP is known to interact with a range of secreted proteins such as apoE, tPA, PAI-1 etc, and so fusing a recognition sequence from one of these proteins for LRP may facilitate transport of the enzyme into the brain, following expression in the liver of the therapeutic protein and secretion into the blood stream (see Gabathuler (2010) Neurobiol Dis. 37(1):48-57).

In certain embodiments, the donor vectors is a vector as shown in SB-IDS AAV (Table 3, SEQ ID NO: 17).

Compositions/Systems

The invention described herein utilizes three AAV vectors for practicing the method. Two vectors are used to deliver the right ZFN and the left ZFN and a third vector is used to provide the IDS donor sequence (see Examples). In certain embodiments, the composition/systems comprising the 3 vectors which includes SB-47171, SB-47898 and SB-IDS AAV.

Cells

Also provided herein are genetically modified cells, for example, liver cells or stem cells comprising a transgene encoding an IDS protein, including cells produced by the methods described herein. The IDS transgene may be expressed extra-chromosomally or can be integrated in a targeted manner into the cell's genome using one or more nucleases. Unlike random integration, nuclease-mediated targeted integration ensures that the transgene is integrated into a specified gene. The transgene may be integrated anywhere in the target gene. In certain embodiments, the transgene is integrated at or near the nuclease binding and/or cleavage site, for example, within 1-300 (or any number of base pairs therebetween) base pairs upstream or downstream of the site of cleavage and/or binding site, more preferably within 1-100 base pairs (or any number of base pairs therebetween) of either side of the cleavage and/or binding site, even more preferably within 1 to 50 base pairs (or any number of base pairs therebetween) of either side of the cleavage and/or binding site. In certain embodiments, the integrated sequence does not include any vector sequences (e.g., viral vector sequences).

Any cell type can be genetically modified as described herein to comprise a transgene, including but not limited to cells or cell lines. Other non-limiting examples of genetically modified cells as described herein include T-cells (e.g., CD4+, CD3+, CD8+, etc.); dendritic cells; B-cells; autologous (e.g., subject-derived). In certain embodiments, the cells are liver cells and are modified in vivo. In certain embodiments, the cells are stem cells, including heterologous pluripotent, totipotent or multipotent stem cells (e.g., CD34+ cells, induced pluripotent stem cells (iPSCs), embryonic stem cells or the like). In certain embodiments, the cells as described herein are stem cells derived from subject.

The cells as described herein are useful in treating and/or preventing MPS

II disease in a subject with the disorder, for example, by in vivo therapies. Ex vivo therapies are also provided, for example when the nuclease-modified cells can be expanded and then reintroduced into the subject using standard techniques. See, e.g., Tebas et al. (2014) New Eng J Med 370(10):901. In the case of stem cells, after infusion into the subject, in vivo differentiation of these precursors into cells expressing the functional protein (from the inserted donor) also occurs.

Pharmaceutical compositions (also referred to as “a formulation” or “article of manufacture” or “drug product” or “set of drug products”) comprising one or more of the compositions (nucleases, IDS donors, cells, etc.) as described herein are also provided. The pharmaceutical compositions may include the same or different types of component compositions in any concentrations. For example, provided herein is an article of manufacture comprising a set of drug products, which include three separate pharmaceutical compositions as follows: a first pharmaceutical composition comprising a purified AAV vector carrying one member of a ZFN pair (e.g., a left ZFN); a second pharmaceutical composition comprising a purified AAV vector carrying the other member of a ZFN pair (e.g., a right ZFN); and a third pharmaceutical composition comprising a purified AAV vector carrying an IDS donor. The left ZFNs may comprise the ZFN designated 47171 (e.g., drug product designated SB-A6P-ZLEF) or the ZFN designated 71557 (e.g., drug product designated SB-A6P-ZL2) and the right ZFN may comprise the ZFN designated 47898 (e.g., drug product designated SB-A6P-ZRIGHT) or the ZFN designated 71728 (e.g., drug product designated SB-A6P-ZL2). One, two or three of the three pharmaceutical compositions may be individually formulated in phosphate buffered saline (PBS) containing CaCl2, MgCl2, NaCl, sucrose and a Poloxamer (e.g., Poloxamer P188) or in a Normal Saline (NS) formulation. In some embodiments, the composition comprises phosphate buffered saline (PBS) comprising approximately 1.15 mg/ML of sodium phosphate, 0.2 mg/mL potassium phosphate, 8.0 mg/mL sodium chloride, 0.2 mg/mL potassium chloride, 0.13 mg/mL calcium chloride, and 0.1 mg/mL Magnesium chloride. The PBS is further modified with 2.05 mg/mL sodium chloride, 10-12 mg/mL of sucrose and 0.5 to 0 mg/mL of Kolliphor® (poloxamer or P188). Any concentration of ZFN can be used, including but not limited to the concentrations shown in Table 6. Further, the article of manufacture may include any ratio of the three pharmaceutical compositions can be used, for example 1:1:8 (left ZFN:right ZFN:IDS donor).

The pharmaceutical compositions (article of manufacture/set of drug products) are administered (e.g., intravenously) to a subject in need thereof such that IDS is expressed in the subject, including at therapeutic levels (e.g., in plasma and/or blood leukocytes) for treatment of MPS II. The compositions may be administered separately or, preferably, the article of manufacture comprising a set of three drug products (ZFN1, ZFN2, and IDS donor) are combined prior to administration, for example in an intravenous infusion bag. In addition, these formulations may be cryopreserved prior to administration to a subject.

Delivery

The nucleases, polynucleotides encoding these nucleases, donor polynucleotides and compositions comprising the proteins and/or polynucleotides described herein may be delivered in vivo or ex vivo by any suitable means.

Methods of delivering nucleases as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Nucleases and/or donor constructs as described herein may also be delivered using vectors containing sequences encoding one or more of the zinc finger, TALEN and/or Cas protein(s). Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleases and a donor construct are introduced into the cell, the nucleases and/or donor polynucleotide may be carried on the same vector or on different vectors. When multiple vectors are used, each vector may comprise a sequence encoding one or multiple nucleases and/or donor constructs.

Conventional viral and non-viral based gene transfer methods can be used to introduce nucleic acids encoding nucleases and donor constructs in cells (e.g., mammalian cells) and target tissues. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1988) Biotechnology 6(10):1149-1154; Vigne (1995) Restorative Neurology and Neuroscience 8:35-36; Kremer & Perricaudet (1995) British Medical Bulletin 51(1):31-44; Haddada et al. in Current Topics in Microbiology and Immunology Doerfler and Bohm (eds.) (1995); and Yu et al. (1994) Gene Therapy 1:13-26.

Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024.

The preparation of lipid:nucleic acid complexes, including targeted liposomes such as immunolipid complexes, is well known to one of skill in the art (see, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGenelC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al. (2009) Nature Biotechnology 27(7):643).

The use of RNA or DNA viral based systems for the delivery of nucleic acids encoding engineered ZFPs take advantage of highly evolved processes for targeting a virus to specific cells in the body and trafficking the viral payload to the nucleus. Viral vectors can be administered directly to subjects (in vivo) or they can be used to treat cells in vitro and the modified cells are administered to subjects (ex vivo). Conventional viral based systems for the delivery of ZFPs include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been measured in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; International Patent Publication No. WO 94/26877.

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al. (1987) Virology 160:38-47; U.S. Pat. No. 4,797,368; International Patent Publication No. WO 93/24641; Kotin (1994) Human Gene Therapy 5:793-801; Muzyczka (1994) J. Clin. Invest. 94:1351. Construction of recombinant AAV vectors are described in a number of publications, including U.S. Pat. No. 5,173,414; Tratschin et al. (1985) Mol. Cell. Biol. 5:3251-3260; Tratschin et al. (1984) Mol. Cell. Biol. 4:2072-2081; Hermonat & Muzyczka (1984) PNAS 81:6466-6470; and Samulski et al. (1989) J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al. (1995) Blood 85:3048-305; Kohn et al. (1995) Nat. Med. 1:1017-102; Malech et al. (1997) PNAS 94(22):12133-12138). PA317/pLASN was the first therapeutic vector used in a gene therapy trial. (Blaese et al. (1995) Science 270:475-480). Transduction efficiencies of 50% or greater have been measured for MFG-S packaged vectors. (Ellem et al. (1997) Immunol Immunother. 44(1):10-20; Dranoff et al. (1997) Hum. Gene Ther. 1:111-2.

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al. (1998) Lancet 351(9117):1702-3; Kearns et al. (1996) Gene Ther. 9:748-55). Other AAV serotypes, including by non-limiting example, AAV1, AAV3, AAV4, AAV5, AAV6, AAV8, AAV 8.2, AAV9 and AAV rh10 and pseudotyped AAV such as AAV2/8, AAV2/5 and AAV2/6 can also be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for anti-tumor immunization with intramuscular injection (Sterman et al. (1998) Hum. Gene Ther. 7:1083-9). Additional examples of the use of adenovirus vectors for gene transfer in clinical trials include Rosenecker et al. (1996) Infection 24(1):5-10; Sterman et al. (1998) Hum. Gene Ther. 9(7):1083-1089; Welsh et al. (1995) Hum. Gene Ther. 2:205-18; Alvarez et al. (1997) Hum. Gene Ther. 5:597-613; Topf et al. (1998) Gene Ther. 5:507-513; Sterman et al. (1998) Hum. Gene Ther. 7:1083-1089.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751, reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the vector.

Vectors (e.g., retroviruses, adenoviruses, liposomes, etc.) containing nucleases and/or donor constructs can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of polynucleotides described herein include non-integrating lentivirus vectors (IDLV). See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222; U.S. Patent Publication No 2009/054985.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

It will be apparent that the nuclease-encoding sequences and donor constructs can be delivered using the same or different systems. For example, a donor polynucleotide can be carried by a plasmid, while the one or more nucleases can be carried by an AAV vector. Furthermore, the different vectors can be administered by the same or different routes (intramuscular injection, tail vein injection, other intravenous injection, intraperitoneal administration and/or intramuscular injection. The vectors can be delivered simultaneously or in any sequential order.

Formulations for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

Applications

The methods of this invention contemplate the treatment and/or prevention of MPS II disease (e.g. a lysosomal storage disease). Treatment can comprise insertion of one or more corrective disease-associated genes (e.g., IDS, etc.) into a safe harbor locus (e.g. albumin) in a cell for expression of the needed enzyme(s) and release into the blood stream. Once in the bloodstream, the secreted enzyme may be taken up by cells in the tissues, wherein the enzyme is then taken up by the lysosomes such that the GAGs are broken down. The transgene may encode a protein comprising a codon optimized transgene (e.g., IDS); and/or a transgene in which epitopes may be removed without functionally altering the protein. In some cases, the methods comprise insertion of an episome expressing the corrective enzyme-encoding transgene into a cell for expression of the needed enzyme and release into the blood stream. Insertion into a secretory cell, such as a liver cell for release of the product into the blood stream, is particularly useful. The methods and compositions of the invention also can be used in any circumstance wherein it is desired to supply an IDS transgene encoding one or more therapeutics in a hematopoietic stem cell such that mature cells (e.g., RBCs) derived from these cells contain the therapeutic. These stem cells can be differentiated in vitro or in vivo and may be derived from a universal donor type of cell which can be used for all subjects. Additionally, the cells may contain a transmembrane protein to traffic the cells in the body. Treatment can also comprise use of subject cells containing the therapeutic transgene where the cells are developed ex vivo and then introduced back into the subject. For example, HSC containing a suitable IDS encoding transgene may be inserted into a subject via an autologous bone marrow transplant. Alternatively, stem cells such as muscle stem cells or iPSC which have been edited using with the IDS encoding transgene maybe also injected into muscle tissue.

Thus, this technology may be of use in a condition where a subject is deficient in some protein due to problems (e.g., problems in expression level or problems with the protein expressed as sub- or non-functioning). Particularly useful with this invention is the expression of transgenes to correct or restore functionality in subjects with MPS II disease.

By way of non-limiting examples, production of the defective or missing proteins is accomplished and used to treat MPS II disease. Nucleic acid donors encoding the proteins may be inserted into a safe harbor locus (e.g. albumin or HPRT) and expressed either using an exogenous promoter or using the promoter present at the safe harbor. Alternatively, donors can be used to correct the defective gene in situ. The desired IDS encoding transgene may be inserted into a CD34+ stem cell and returned to a subject during a bone marrow transplant. Finally, the nucleic acid donor maybe be inserted into a CD34+ stem cell at a beta globin locus such that the mature red blood cell derived from this cell has a high concentration of the biologic encoded by the nucleic acid donor. The biologic-containing RBC can then be targeted to the correct tissue via transmembrane proteins (e.g. receptor or antibody). Additionally, the RBCs may be sensitized ex vivo via electrosensitization to make them more susceptible to disruption following exposure to an energy source (see International Patent Publication No. WO 2002/007752).

In some applications, an endogenous gene may be knocked out by use of the methods and compositions of the invention. Examples of this aspect include knocking out an aberrant gene regulator or an aberrant disease associated gene. In some applications, an aberrant endogenous gene may be replaced, either functionally or in situ, with a wild type version of the gene. The inserted gene may also be altered to improve the expression and/or functionality of the therapeutic IDS protein or to reduce its immunogenicity. In some applications, the inserted IDS encoding transgene is a fusion protein to increase its transport into a selected tissue such as the brain.

In some applications, provided herein is a method of improving or maintaining (slowing the decline) of functional ability in a human subject having MPS II as compared with a subject that has not been treated with the methods and compositions of the invention. In other applications, provided herein is a method of decreasing the need (dose level or frequency) for ERT in a subject with MPS II as compared with a subject that has not been treated with the methods and compositions of the invention. In yet another aspect, provided herein is a method of delaying the need for ERT initiation in a subject with MPS II as compared with a subject that has not been treated with the methods and compositions of the invention. In one aspect, provided herein is a method to delay, reduce or eliminate the need for supportive surgery in a subject with MPS II, comprising treating the subject with the compositions of the invention, as compared to a subject that has not received the compositions. In another aspect, provided herein is a method of delaying, reducing or preventing the need for a bone marrow transplant in a subject with MPS II as compared with a subject that has not been treated with the methods and compositions of the invention. In yet another aspect, provided herein is a method of improving the functional (delaying decline, maintenance) ability in a subject with MPS II by treating the subject with a standard dosing regimen of ERT in combination with treatment with a composition as described herein as compared with a subject that has not been treated with the methods and compositions of the invention. In another aspect, provided herein is a method of suppressing disability progression in a human subject having MPS II as compared with a subject that has not been treated with the methods and compositions of the invention. In yet another aspect, provided herein is a method of delaying, reducing or preventing the need for the use of a medical ventilator device in a subject with MPS II as compared with a subject that has not been treated with the methods and compositions of the invention. In another aspect, provided herein is a method of delaying onset of confirmed disability progression or reducing the risk of confirmed disability progression in a human subject having MPS II as compared to a subject that that has not been treated with the methods and compositions of the invention. In one aspect of the invention, provided herein is a method of reducing, stabilizing or maintaining urine GAGs in a subject with MPS II, comprising treating the subject with the composition of the invention. In yet another aspect, provided herein is a method of extending life expectancy in a subject with MPS II as compared with a subject that has not been treated with the methods and compositions of the invention.

The following Examples relate to exemplary embodiments of the present disclosure in which the nuclease comprises a zinc finger nuclease (ZFN) or TALEN. It will be appreciated that this is for purposes of exemplification only and that other nucleases or nuclease systems can be used, for instance homing endonucleases (meganucleases) with engineered DNA-binding domains and/or fusions of naturally occurring of engineered homing endonucleases (meganucleases) DNA-binding domains and heterologous cleavage domains and/or a CRISPR/Cas system comprising an engineered single guide RNA.

### EXAMPLES

### Example 1

The preparation of polynucleotides and AAV vector comprising the polynucleotides is as follows: The AAV2/6 vector encoding the SB-47171 ZFN (left ZFN) comprises several structural features: the 5′ and 3′ ITRs of the AAV vector, the ApoE/hAAT hepatic control region and α1-anti-trypsin promoter, the human β-globin-IgG chimeric intron, the nuclear localization sequence, the ZFP 47171 ZFN binding domain, the FokI ELD nuclease domain, and a polyadenylation signal. The locations of the various elements are shown below in Table 1.

TABLE 1
Elements of SB-47171 AAV (SEQ ID NO: 9)

| | | | SEQ ID |
|---|---|---|---|
| Feature | Description | Position-annotation | NO |
| ITR | 5′ inverted terminal repeat | 1-130-[plain text in brackets] | 1 |
| ApoE/hAAT | ApoE Hepatic Control Region & α1-antitrypsin | 141-863-underlined | 2 |
| | promoter | | |
| Chimeric | Human β globin-IgG chimeric intron | 867-999-italics | 3 |
| Intron | | | |
| NLS | NLS | 1016-1036-double underline | 4 |
| 47171 | ZFP 47171 DNA-binding domain | 1055-1486-Bold | 5 |
| Fokl-ELD | Fokl-ELD nuclease domain | 1493-2092-lower case | 6 |
| poly A | Polyadenylation signal | 2148-2370-dashed underline | 7 |
| ITR | 3′ inverted terminal repeat | 2422-2529-wavy underline | 8 |

The complete nucleotide sequence for the SB-47171 AAV2/6 vector is shown below. The specific annotations shown in Table 1 are indicated in the sequence text as shown in Table 1:

(SEQ ID NO: 9)

| | |
|---|---|
| [CTGCGCGCTC GCTCGCTCAC TGAGGCCGCC CGGGCAAAGC CCGGGCGTCG | 50 |
| GGCGACCTTT GGTCGCCCGG CCTCAGTGAG CGAGCGAGCG CGCAGAGAGG | 100 |
| GAGTGGCCAA CTCCATCACT AGGGGTTCCT]GCGGCCTAGT AGGCTCAGAG | 150 |
| GCACACAGGA GTTTCTGGGC TCACCCTGCC CCCTTCCAAC CCCTCAGTTC | 200 |
| CCATCCTCCA GCAGCTGTTT GTGTGCTGCC TCTGAAGTCC ACACTGAACA | 250 |
| AACTTCAGCC TACTCATGTC CCTAAAATGG GCAAACATTG CAAGCAGCAA | 300 |
| ACAGCAAACA CACAGCCCTC CCTGCCTGCT GACCTTGGAG CTGGGGCAGA | 350 |
| GGTCAGAGAC CTCTCTGGGC CCATGCCACC TCCAACATCC ACTCGACCCC | 400 |
| TTGGAATTTC GGTGGAGAGG AGCAGAGGTT GTCCTGGCGT GGTTTAGGTA | 450 |
| GTGTGAGAGG GGTACCCGGG GATCTTGCTA CCAGTGGAAC AGCCACTAAG | 500 |
| GATTCTGCAG TGAGAGCAGA GGGCCAGCTA AGTGGTACTC TCCCAGAGAC | 550 |
| TGTCTGACTC ACGCCACCCC CTCCACCTTG GACACAGGAC GCTGTGGTTT | 600 |
| CTGAGCCAGG TACAATGACT CCTTTCGGTA AGTGCAGTGG AAGCTGTACA | 650 |
| CTGCCCAGGC AAAGCGTCCG GGCAGCGTAG GCGGGCGACT CAGATCCCAG | 700 |
| CCAGTGGACT TAGCCCCTGT TTGCTCCTCC GATAACTGGG GTGACCTTGG | 750 |
| TTAATATTCA CCAGCAGCCT CCCCCGTTGC CCCTCTGGAT CCACTGCTTA | 800 |
| AATACGGACG AGGACAGGGC CCTGTCTCCT CAGCTTCAGG CACCACCACT | 850 |
| GACCTGGGAC AGTCAGGTAA GTATCAAGGT TACAAGACAG GTTTAAGGAG | 900 |
| ACCAATAGAA ACTGGGCTTG TCGAGACAGA GAAGACTCTT GCGTTTCTGA | 950 |
| TAGGCACCTA TTGGTCTTAC TGACATCCAC TTTGCCTTTC TCTCCACAGG | 1000 |
| CAATTCGCCA TGGCCCCCAA GAAGAAGAGG AAGGTGGGCA TCCACGGGGT | 1050 |
| ACCGGCCGCA ATGGCAGAAC GGCCCTTCCA GTGCCGCATC TGCATGCGCA | 1100 |
| ACTTCAGCCA GTCGGGCAAC CTGTCCCGCC ACATCCGGAC TCATACCGGC | 1150 |
| GAAAAACCAT TCGCTTGTGA CATCTGCGGA AGAAAGTTTG CGCTGAAGCA | 1200 |
| GAACCTCTGC ATGCATACCA AGATTCACAC CGGAGAGAAG CCGTTTCAGT | 1250 |
| GTCGCATTTG CATGAGAAAG TTCGCCTGGG CCGATAACCT TCAGAATCAC | 1300 |
| ACCAAGATCC ACACCGGGGA AAAGCCGTTC CAGTGCCGGA TCTGCATGAG | 1350 |
| GAACTTCTCA ACGTCCGGAA ACCTGACCAG GCATATCCGG ACCCACACTG | 1400 |
| GGGAGAAGCC TTTCGCCTGC GACATTTGCG GTCGGAAGTT CGCCCGGCAA | 1450 |
| TCCCACTTGT GTCTCCACAC TAAGATCCAC CTGAGAGGAT CCcagctggt | 1500 |
| gaagagcgag ctggaggaga agaagtccga gctgcggcac aagctgaagt | 1550 |
| acgtgcccca cgagtacatc gagctgatcg agatcgccag gaacagcacc | 1600 |
| caggaccgca tcctggagat gaaggtgatg gagttcttca tgaaggtgta | 1650 |
| cggctacagg ggaaagcacc tgggcggaag cagaaagcct gacggcgcca | 1700 |
| tctatacagt gggcagcccc atcgattacg gcgtgatcgt ggacacaaag | 1750 |
| gcctacagcg gcggctacaa tctgcctatc ggccaggccg acgagatgga | 1800 |
| gagatacgtg gaggagaacc agacccggga taagcacctc aaccccaacg | 1850 |
| agtggtggaa ggtgtaccct agcagcgtga ccgagttcaa gttcctgttc | 1900 |
| gtgagcggcc acttcaaggg caactacaag gcccagctga ccaggctgaa | 1950 |
| ccacatcacc aactgcaatg gcgccgtgct gagcgtggag gagctgctga | 2000 |
| tcggcggcga gatgatcaaa gccggcaccc tgacactgga ggaggtgcgg | 2050 |
| cgcaagttca acaacggcga gatcaacttc agatcttgat aaCTCGAGTC | 2100 |
| 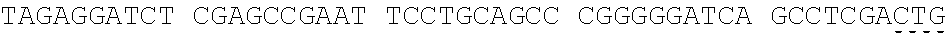 | 2150 |
| 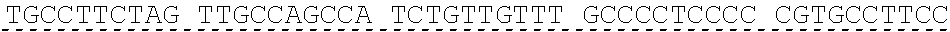 | 2200 |
| 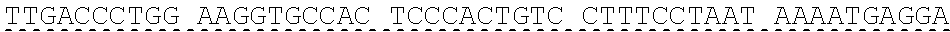 | 2250 |
| 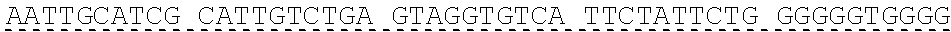 | 2300 |
| 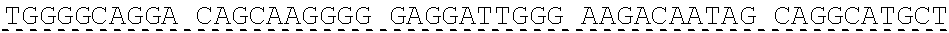 | 2350 |
| 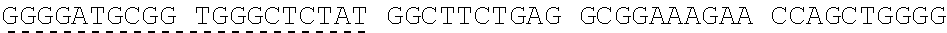 | 2400 |
| 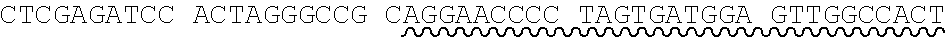 | 2450 |
| 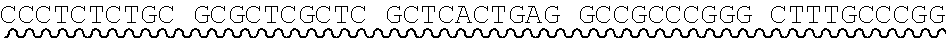 | 2500 |
| 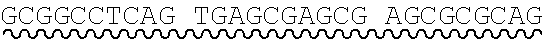 | 2529 |

The AAV2/6 vector comprising SB-47898 similarly comprises several features, and these are shown below in Table 2.

TABLE 2
Elements of SB-47898 AAV (SEQ ID NO: 12)

| Feature | Description | Position-annotation | SEQ ID NO: |
|---|---|---|---|
| ITR | 5′ inverted terminal repeat | 1-130-[plain text in brackets] | 1 |
| ApoE/hAAT | ApoE Hepatic Control Region & α1-antitrypsin | 141-863 underlined | 2 |
| | promoter | | |
| Chimeric Intron | Human β globin-IgG chimeric intron | 867-999 italics | 3 |
| NLS | NLS | 1016-1036 double underline | 4 |
| 47898 | ZFP 47898 DNA-binding domain | 1055-1570 Bold | 10 |
| Fokl-KKR | Fokl-KKR nuclease domain | 1577-2170 lower case | 11 |
| poly A | Polyadenylation signal | 2226-2448 dashed underline | 7 |
| ITR | 3′ inverted terminal repeat | 2500-2607 wavy underline | 8 |

The complete nucleotide sequence for the SB-47898 AAV2/6 vector is shown below. The specific annotations shown in Table 2 are indicated in the sequence text as shown in Table 2.

(SEQ ID NO: 12)

| | |
|---|---|
| [CTGCGCGCTC GCTCGCTCAC TGAGGCCGCC CGGGCAAAGC CCGGGCGTCG | 50 |
| GGCGACCTTT GGTCGCCCGG CCTCAGTGAG CGAGCGAGCG CGCAGAGAGG | 100 |
| GAGTGGCCAA CTCCATCACT AGGGGTTCCT]GCGGCCTAGT AGGCTCAGAG | 150 |
| GCACACAGGA GTTTCTGGGC TCACCCTGCC CCCTTCCAAC CCCTCAGTTC | 200 |
| CCATCCTCCA GCAGCTGTTT GTGTGCTGCC TCTGAAGTCC ACACTGAACA | 250 |
| AACTTCAGCC TACTCATGTC CCTAAAATGG GCAAACATTG CAAGCAGCAA | 300 |
| ACAGCAAACA CACAGCCCTC CCTGCCTGCT GACCTTGGAG CTGGGGCAGA | 350 |
| GGTCAGAGAC CTCTCTGGGC CCATGCCACC TCCAACATCC ACTCGACCCC | 400 |
| TTGGAATTTC GGTGGAGAGG AGCAGAGGTT GTCCTGGCGT GGTTTAGGTA | 450 |
| GTGTGAGAGG GGTACCCGGG GATCTTGCTA CCAGTGGAAC AGCCACTAAG | 500 |
| GATTCTGCAG TGAGAGCAGA GGGCCAGCTA AGTGGTACTC TCCCAGAGAC | 550 |
| TGTCTGACTC ACGCCACCCC CTCCACCTTG GACACAGGAC GCTGTGGTTT | 600 |
| CTGAGCCAGG TACAATGACT CCTTTCGGTA AGTGCAGTGG AAGCTGTACA | 650 |
| CTGCCCAGGC AAAGCGTCCG GGCAGCGTAG GCGGGCGACT CAGATCCCAG | 700 |
| CCAGTGGACT TAGCCCCTGT TTGCTCCTCC GATAACTGGG GTGACCTTGG | 750 |
| TTAATATTCA CCAGCAGCCT CCCCCGTTGC CCCTCTGGAT CCACTGCTTA | 800 |
| AATACGGACG AGGACAGGGC CCTGTCTCCT CAGCTTCAGG CACCACCACT | 850 |
| GACCTGGGAC AGTCAGGTAA GTATCAAGGT TACAAGACAG GTTTAAGGAG | 900 |
| ACCAATAGAA ACTGGGCTTG TCGAGACAGA GAAGACTCTT GCGTTTCTGA | 950 |
| TAGGCACCTA TTGGTCTTAC TGACATCCAC TTTGCCTTTC TCTCCACAGG | 1000 |
| CAATTCGCCA TGGCCCCCAA GAAGAAGAGG AAGGTGGGCA TCCACGGGGT | 1050 |
| ACCGGCCGCA ATGGCAGAGA GGCCCTTTCA GTGCCGGATC TGCATGCGGA | 1100 |
| ACTTCTCCAC CCCACAACTT CTGGACCGAC ATATCCGCAC CCATACCGGG | 1150 |
| GAAAAGCCTT TCGCGTGCGA CATTTGCGGA CGGAAATTCG CGTTGAAGCA | 1200 |
| CAATCTCCTG ACCCACACTA AGATTCATAC TGGCGAAAAG CCGTTCCAGT | 1250 |
| GCCGCATCTG TATGAGGAAC TTCAGCGATC AGTCGAACCT GAACGCCCAC | 1300 |
| ATTCGGACTC ATACCGGAGA AAAGCCCTTT GCCTGCGATA TCTGCGGTCG | 1350 |
| CAAGTTCGCT AGGAACTTCT CACTGACCAT GCACACCAAA ATCCACACTG | 1400 |
| GAGAGCGGGG ATTCCAGTGT AGAATCTGTA TGCGCAACTT CTCCCTGCGG | 1450 |
| CACGACCTGG ACCGCCACAT CAGAACCCAC ACCGGGGAGA AGCCGTTCGC | 1500 |
| CTGCGACATC TGCGGCCGGA AGTTCGCCCA CCGGTCCAAC CTGAACAAGC | 1550 |
| ACACGAAGAT TCACCTCCGC GGATCCcagc tggtgaagag cgagctggag | 1600 |
| gagaagaagt ccgagctgcg gcacaagctg aagtacgtgc cccacgagta | 1650 |
| catcgagctg atcgagatcg ccaggaacag cacccaggac cgcatcctgg | 1700 |
| agatgaaggt gatggagttc ttcatgaagg tgtacggcta caggggaaag | 1750 |
| cacctgggcg gaagcagaaa gcctgacggc gccatctata cagtgggcag | 1800 |
| ccccatcgat tacggcgtga tcgtggacac aaaggcctac agcggcggct | 1850 |
| acaatctgcc tatcggccag gccgacgaga tgcagagata cgtgaaggag | 1900 |
| aaccagaccc ggaataagca catcaacccc aacgagtggt ggaaggtgta | 1950 |
| ccctagcagc gtgaccgagt tcaagttcct gttcgtgagc ggccacttca | 2000 |
| agggcaacta caaggcccag ctgaccaggc tgaaccgcaa aaccaactgc | 2050 |
| aatggcgccg tgctgagcgt ggaggagctg ctgatcggcg gcgagatgat | 2100 |
| caaagccggc accctgacac tggaggaggt gcggcgcaag ttcaacaacg | 2150 |
| gcgagatcaa cttctgataa CTCGAGTCTA GAGGATCTCG AGCCGAATTC | 2200 |
| 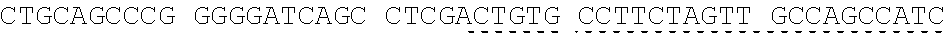 | 2250 |
| 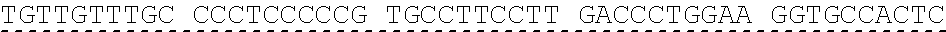 | 2300 |
| 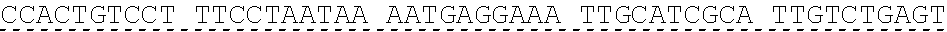 | 2350 |
| 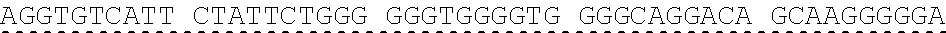 | 2400 |
| 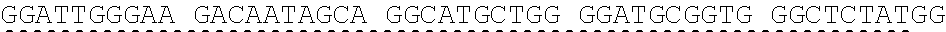 | 2450 |
| 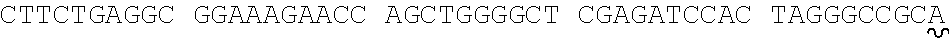 | 2500 |
| 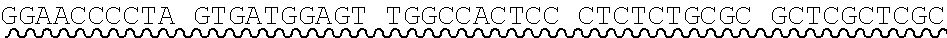 | 2550 |
| 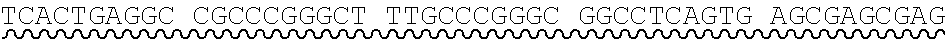 | 2600 |
| 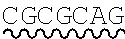 | 2607 |

The AAV2/6 vector encoding the SB-IDS transgene donor comprises several structural features: the 5′ and 3′ ITRs of the AAV vector, left and right homology arms (LA and RA) that have homology to the regions flanking the targeted cleavage site in the albumin gene, a splice acceptor derived from the human Factor IX exon 2 splice acceptor to ensure efficient joining of the transgene sequence to the albumin promoter, a codon optimized hIDS cDNA sequence, and a polyadenylation signal sequence. The locations of the various elements are shown below in Table 3.

TABLE 3
Elements of SB-IDS AAV (SEQ ID NO: 17)

| | | | SEQ |
|---|---|---|---|
| Feature | Description | Position-annotation | ID NO |
| ITR | 5′ inverted terminal | 1-130-[plain text in brackets] | 1 |
| | repeat | | |
| LA | Left homology arm | 271-550-underline | 13 |
| SA | Splice acceptor | 557-584-italics | 14 |
| hIDS | Codon optimized hIDS | 587-2161-Bold | 15 |
| | cDNA | | |
| poly A | Polyadenylation signal | 2174-2398-dashed underline | 7 |
| RA | Right homology arm | 2405-2504-wavy underline | 16 |
| ITR | 3′ inverted terminal | 2651-2758-double underline | 8 |
| | repeat | | |

The complete nucleotide sequence for the SB-IDS AAV2/6 vector is shown below. The specific annotations shown in Table 3 are indicated in the sequence text as shown in Table 3.

(SEQ ID NO: 17)

| | |
|---|---|
| [CTGCGCGCTCGCTCGCTCAC TGAGGCCGCC CGGGCAAAGC CCGGGCGTCG | 50 |
| GGCGACCTTT GGTCGCCCGG CCTCAGTGAG CGAGCGAGCG CGCAGAGAGG | 100 |
| GAGTGGCCAA CTCCATCACT AGGGGTTCCT]GCGGCCTAAG CTTGAGCGGA | 150 |
| GTTCCAATTG TACTGTACAG AACCATGGTC ACATGTTTAA CGCTAGCGTG | 200 |
| CCGACCTGGT AAACTGATCA GTGGGTGCAC TTAGGACTGC GTCTTACGCT | 250 |
| AATCACATGC GTGCGGCCGC TTTATTCTAT TTTCCCAGTA AAATAAAGTT | 300 |
| TTAGTAAACT CTGCATCTTT AAAGAATTAT TTTGGCATTT ATTTCTAAAA | 350 |
| TGGCATAGTA TTTTGTATTT GTGAAGTCTT ACAAGGTTAT CTTATTAATA | 400 |
| AAATTCAAAC ATCCTAGGTA AAAAAAAAAA AAGGTCAGAA TTGTTTAGTG | 450 |
| ACTGTAATTT TCTTTTGCGC ACTAAGGAAA GTGCAAAGTA ACTTAGAGTG | 500 |
| ACTGAAACTT CACAGAATAG GGTTGAAGAT TGAATTCATA ACTATCCCAA | 550 |
| GGTACCACTA AAGAATTATT CTTTTACATT TCAGTTAGCG AAACCCAGGC | 600 |
| CAACTCAACT ACAGATGCGC TTAACGTCCT GCTCATCATC GTGGACGATT | 650 |
| TGCGGCCGTC GCTTGGCTGC TATGGAGATA AGCTCGTCCG CTCGCCGAAC | 700 |
| ATCGATCAGT TGGCCTCACA CTCACTGCTT TTCCAAAATG CGTTTGCGCA | 750 |
| GCAGGCTGTC TGTGCACCTT CAAGAGTCTC ATTCTTGACC GGGCGACGCC | 800 |
| CTGACACAAC GCGGCTGTAC GACTTCAACA GCTACTGGAG AGTCCACGCG | 850 |
| GGTAACTTTT CAACTATCCC ACAGTACTTT AAAGAGAACG GATACGTGAC | 900 |
| AATGAGCGTG GGAAAGGTCT TTCACCCCGG CATCTCCTCG AATCACACCG | 950 |
| ACGATTCGCC CTACTCGTGG TCGTTTCCTC CCTACCATCC TTCGAGCGAG | 1000 |
| AAGTATGAGA ACACGAAAAC TTGTCGCGGA CCCGACGGAG AGCTGCACGC | 1050 |
| TAATCTGCTG TGTCCGGTGG ATGTCTTGGA CGTGCCCGAG GGAACGCTCC | 1100 |
| CCGACAAGCA GTCAACGGAG CAGGCGATTC AGTTGCTGGA GAAGATGAAA | 1150 |
| ACAAGCGCGT CGCCTTTCTT CCTCGCCGTG GGGTATCACA AGCCCCATAT | 1200 |
| TCCTTTCCGC TACCCGAAGG AGTTCCAGAA ACTTTATCCT TTGGAAAACA | 1250 |
| TCACTTTGGC ACCGGACCCG GAAGTCCCCG ACGGTCTGCC ACCCGTGGCC | 1300 |
| TACAATCCCT GGATGGATAT CAGGCAGAGG GAAGATGTGC AGGCACTCAA | 1350 |
| CATCTCAGTC CCCTACGGGC CTATTCCAGT CGATTTTCAA CGCAAGATTC | 1400 |
| GGCAGTCGTA TTTTGCGTCG GTGTCCTACC TCGATACGCA AGTAGGTCGA | 1450 |
| CTTCTGAGCG CGCTTGATGA CCTTCAGCTG GCAAATTCCA CAATCATCGC | 1500 |
| CTTTACGTCG GACCATGGGT GGGCGTTGGG AGAGCATGGA GAGTGGGCAA | 1550 |
| AGTATAGCAA TTTTGATGTA GCAACGCACG TGCCCCTGAT TTTCTACGTG | 1600 |
| CCGGGTAGAA CGGCCTCGCT TCCCGAGGCA GGCGAAAAAC TTTTTCCCTA | 1650 |
| TCTCGATCCA TTCGACTCGG CGAGCCAGCT TATGGAACCG GGCAGACAAT | 1700 |
| CCATGGACTT GGTAGAATTG GTGTCCCTTT TTCCGACCCT CGCCGGGTTG | 1750 |
| GCGGGCTTGC AAGTACCCCC TAGATGCCCT GTACCGAGCT TCCATGTGGA | 1800 |
| ACTCTGCCGC GAAGGGAAAA ACCTCCTCAA ACACTTTCGG TTCAGGGACC | 1850 |
| TTGAGGAGGA CCCCTATCTG CCAGGGAATC CGCGAGAGTT GATTGCCTAT | 1900 |
| TCCCAGTATC CGCGACCCAG CGATATTCCT CAATGGAACT CCGATAAGCC | 1950 |
| CTCCCTCAAA GACATCAAGA TTATGGGGTA CTCGATCAGG ACCATCGACT | 2000 |
| ATCGCTACAC AGTGTGGGTA GGGTTCAATC CTGACGAATT CCTCGCGAAC | 2050 |
| TTTTCGGACA TCCACGCTGG TGAGCTGTAT TTCGTAGACT CGGACCCGTT | 2100 |
| GCAAGATCAC AATATGTATA ATGATTCCCA AGGAGGAGAT TTGTTCCAGC | 2150 |
| 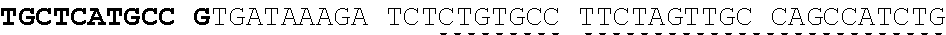 | 2200 |
| 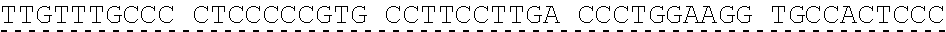 | 2250 |
| 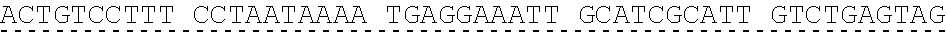 | 2300 |
| 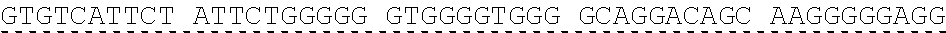 | 2350 |
| 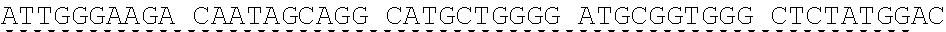 | 2400 |
| 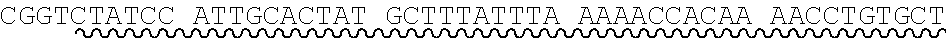 | 2450 |
| 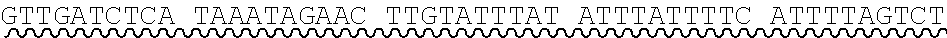 | 2500 |
| 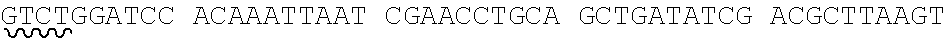 | 2550 |
| AGGGCTTAGC AAACGCGTCT CCAACGTTTC GCCGTTAACA CCCCACATAG | 2600 |
| TGAGTGGTCT TAGTAGTCCG GGTGTTTAAA CTGAAAGATA ACTCGAGCGC | 2650 |
| AGGAACCCCT AGTGATGGAG TTGGCCACTC CCTCTCTGCG CGCTCGCTCG | 2700 |
| CTCACTGAGG CCGCCCGGGC TTTGCCCGGG CGGCCTCAGT GAGCGAGCGA | 2750 |
| GCGCGCAG | 2758 |

The complete nucleotide sequence for the SB-71557 AAV2/6 vector is shown below. The specific annotations shown in Table 4 are indicated in the sequence text as shown in Table 4.

TABLE 4
Elements of SB71557 AAV (SEQ ID NO: 30)

| Nucleotide | | SEQ | |
|---|---|---|---|
| Position- | Feature/ | ID | |
| annotation | Description | NO: | Sequence |
| 1-130 | 5′ ITR | 1 | CTGCGCGCTCGCTCGCTCACTGAGGCCGCCCGGGCAAAGCCCGG |
| | [plain text in | | GCGTCGGGCGACCTTTGGTCGCCCGGCCTCAGTGAGCGAGCGAG |
| | brackets] | | CGCGCAGAGAGGGAGTGGCCAACTCCATCACTAGGGGTTCCT |
| 156-476 | ApoE | 32 | AGGCTCAGAGGCACACAGGAGTTTCTGGGCTCACCCTGCCCCCT |
| | (Enhancer) | | TCCAACCCCTCAGTTCCCATCCTCCAGCAGCTGTTTGTGTGCTG |
| | underlined | | CCTCTGAAGTCCACACTGAACAAACTTCAGCCTACTCATGTCCC |
| | | | TAAAATGGGCAAACATTGCAAGCAGCAAACAGCAAACACACAGC |
| | | | CCTCCCTGCCTGCTGACCTTGGAGCTGGGGCAGAGGTCAGAGAC |
| | | | CTCTCTGGGCCCATGCCACCTCCAACATCCACTCGACCCCTTGG |
| | | | AATTTCGGTGGAGAGGAGCAGAGGTTGTCCTGGCGTGGTTTAGG |
| | | | TAGTGTGAGAGGG |
| 485-877 | hAAT | 33 | GATCTTGCTACCAGTGGAACAGCCACTAAGGATTCTGCAGTGAG |
| | (Promoter) | | AGCAGAGGGCCAGCTAAGTGGTACTCTCCCAGAGACTGTCTGAC |
| | italics | | TCACGCCACCCCCTCCACCTTGGACACAGGACGCTGTGGTTTCT |
| | | | GAGCCAGGTACAATGACTCCTTTCGGTAAGTGCAGTGGAAGCTG |
| | | | TACACTGCCCAGGCAAAGCGTCCGGGCAGCGTAGGCGGGCGACT |
| | | | CAGATCCCAGCCAGTGGACTTAGCCCCTGTTTGCTCCTCCGATA |
| | | | ACTGGGGTGACCTTGGTTAATATTCACCAGCAGCCTCCCCCGTT |
| | | | GCCCCTCTGGATCCACTGCTTAAATACGGACGAGGACAGGGCCC |
| | | | TGTCTCCTCAGCTTCAGGCACCACCACTGACCTGGGACAGT |
| 886-933 | 5′ UTR | 18 | CTTGTTCTTTTTGCAGAAGCTCAGAATAAACGCTCAACTTTGGC |
| | Bold | | A GAT |
| 943-1075 | Human β | 3 | GTAAGTATCAAGGTTACAAGACAGGTTTAAGGAGACCAATAGAA |
| | globin/IgG | | ACTGGGCTTGTCGAGACAGAGAAGACTCTTGCGTTTCTGATAGG |
| | chimeric | | CACCTATTGGTCTTACTGACATCCACTTTGCCTTTCTCTCCACA |
| | intron | | G |
| | (Intron) | | |
| | double | | |
| | underlined | | |
| 1086-1154 | N-terminal | 34 | GACTACAAAGACCATGACGGTGATTATAAAGATCATGACATCGA |
| | peptide | | TTACAAGGATGACGATGACAAG |
| 1161-1181 | Nuclear | 36 | CCCAAGAAGAAGAGGAAGGTC |
| | localization | | |
| | signal | | |
| | Bold italic | | |
| 1200-1631 | ZFP 71557 | 28 | GCCGCTATGGCTGAGAGGCCCTTCCAGTGTCGAATCTGCATGCA |
| | DNA- | | GAACTTCAGTCAGTCCGGCAACCTGGCCCGCCACATCCGCACCC |
| | binding | | ACACCGGCGAGAAGCCTTTTGCCTGTGACATTTGTGGGAGGAAA |
| | domain | | TTTGCCCTGAAGCAGAACCTGTGTATGCATACCAAGATACACAC |
| | lower case | | GGGCGAGAAGCCCTTCCAGTGTCGAATCTGCATGCAGAAGTTTG |
| | | | CCTGGCAGTCCAACCTGCAGAACCATACCAAGATACACACGGGC |
| | | | GAGAAGCCCTTCCAGTGTCGAATCTGCATGCGTAACTTCAGTAC |
| | | | CTCCGGCAACCTGACCCGCCACATCCGCACCCACACCGGCGAGA |
| | | | AGCCTTTTGCCTGTGACATTTGTGGGAGGAAATTTGCCCGCCGC |
| | | | TCCCACCTGACCTCCCATACCAAGATACACCTGCGG |
| 1638-2237 | FokI-ELD | 38 | CAGCTGGTGAAGAGCGAGCTGGAGGAGAAGAAGTCCGAGCTGCG |
| | nuclease | | GCACAAGCTGAAGTACGTGCCCCACGAGTACATCGAGCTGATCG |
| | domain | | AGATCGCCAGGAACAGCACCCAGGACCGCATCCTGGAGATGAAG |
| | N542D | | GTGATGGAGTTCTTCATGAAGGTGTACGGCTACAGGGGAAAGCA |
| | Dashed | | CCTGGGCGGAAGCAGAAAGCCTGACGGCGCCATCTATACAGTGG |
| | underlined | | GCAGCCCCATCGATTACGGCGTGATCGTGGACACAAAGGCCTAC |
| | | | AGCGGCGGCTACAATCTGCCTATCGGCCAGGCCGACGAGATGGA |
| | | | GAGATACGTGGAGGAGAACCAGACCCGGGATAAGCACCTCAACC |
| | | | CCAACGAGTGGTGGAAGGTGTACCCTAGCAGCGTGACCGAGTTC |
| | | | AAGTTCCTGTTCGTGAGCGGCCACTTCAAGGGCAACTACAAGGC |
| | | | CCAGCTGACC |
| | | | AGGCTGAACCACATCACCAACTGCGACGGCGCCGTGCTGAGCGT |
| | | | GGAGGAGCTGCTGATCGGCGGCGAGATGATCAAAGCCGGCACCC |
| | | | TGACACTGGAGGAGGTGCGGCGCAAGTTCAACAACGGCGAGATC |
| | | | AACTTCAGATCTTGATAA |
| 2250-2841 | WPREmut6 | 37 | AATCAACCTCTGGATTACAAAATTTGTGAAAGATTGACTGATAT |
| | 3′UTR | | TCTTAACTATGTTGCTCCTTTTACGCTGTGTGGATATGCTGCTT |
| | Dotted | | TAATGCCTCTGTATCATGCTATTGCTTCCCGTACGGCTTTCGTT |
| | underlined | | TTCTCCTCCTTGTATAAATCCTGGTTGCTGTCTCTTTATGAGGA |
| | | | GTTGTGGCCCGTTGTCCGTCAACGTGGCGTGGTGTGCTCTGTGT |
| | | | TTGCTGACGCAACCCCCACTGGCTGGGGCATTGCCACCACCTGT |
| | | | CAACTCCTTTCTGGGACTTTCGCTTTCCCCCTCCCGATCGCCAC |
| | | | GGCAGAACTCATCGCCGCCTGCCTTGCCCGCTGCTGGACAGGGG |
| | | | CTAGGTTGCTGGGCACTGATAATTCCGTGGTGTTGTCGGGGAAA |
| | | | TCATCGTCCTTTCCTTGGCTGCTCGCCTGTGTTGCCAACTGGAT |
| | | | CCTGCGCGGG |
| | | | ACGTCCTTCTGCTACGTCCCTTCGGCTCTCAATCCAGCGGACCT |
| | | | CCCTTCCCGAGGCCTTCTGCCGGTTCTGCGGCCTCTCCCGCGTC |
| | | | TTCGCTTTCGGCCTCCGACGAGTCGGATCTCCCTTTGGGCCGCC |
| | | | TCCCCGCCTG |
| 2848-3070 | Poly- | 7 | CTGTGCCTTCTAGTTGCCAGCCATCTGTTGTTTGCCCCTCCCCC |
| | adenylation | | GTGCCTTCCTTGACCCTGGAAGGTGCCACTCCCACTGTCCTTTC |
| | signal | | CTAATAAAATGAGGAAATTGCATCGCATTGTCTGAGTAGGTGTC |
| | | | ATTCTATTCTGGGGGGTGGGGTGGGGCAGGACAGCAAGGGGGAG |
| | | | GATTGGGAAGACAATAGCAGGCATGCTGGGGATGCGGTGGGCTC |
| | | | TAT |
| 3088-3195 | 3′ ITR | 8 | AGGAACCCCTAGTGATGGAGTTGGCCACTCCCTCTCTGCGCGCT |
| | [Bold text | | CGCTCGCTCACTGAGGCCGCCCGGGCTTTGCCCGGGCGGCCTCA |
| | in brackets] | | GTGAGCGAGCGAGCGCGCAG |

### Complete Sequence of 71557 AAV:

(SEQ ID NO: 30)

| | |
|---|---|
| [CTGCGCGCTC GCTCGCTCAC TGAGGCCGCC CGGGCAAAGC CCGGGCGTCG | 50 |
| GGCGACCTTT GGTCGCCCGG CCTCAGTGAG CGAGCGAGCG CGCAGAGAGG | 100 |
| GAGTGGCCAA CTCCATCACT AGGGGTTCCT] GCGGCCTAAG CTTGAGCTCT | 150 |
| TCGAAAGGCT CAGAGGCACA CAGGAGTTTC TGGGCTCACC CTGCCCCCTT | 200 |
| CCAACCCCTC AGTTCCCATC CTCCAGCAGC TGTTTGTGTG CTGCCTCTGA | 250 |
| AGTCCACACT GAACAAACTT CAGCCTACTC ATGTCCCTAA AATGGGCAAA | 300 |
| CATTGCAAGC AGCAAACAGC AAACACACAG CCCTCCCTGC CTGCTGACCT | 350 |
| TGGAGCTGGG GCAGAGGTCA GAGACCTCTC TGGGCCCATG CCACCTCCAA | 400 |
| CATCCACTCG ACCCCTTGGA ATTTCGGTGG AGAGGAGCAG AGGTTGTCCT | 450 |
| GGCGTGGTTT AGGTAGTGTG AGAGGGGTCC CGGGGATCTT GCTACCAGTG | 500 |
| GAACAGCCAC TAAGGATTCT GCAGTGAGAG CAGAGGGCCA GCTAAGTGGT | 550 |
| ACTCTCCCAG AGACTGTCTG ACTCACGCCA CCCCCTCCAC CTTGGACACA | 600 |
| GGACGCTGTG GTTTCTGAGC CAGGTACAAT GACTCCTTTC GGTAAGTGCA | 650 |
| GTGGAAGCTG TACACTGCCC AGGCAAAGCG TCCGGGCAGC GTAGGCGGGC | 700 |
| GACTCAGATC CCAGCCAGTG GACTTAGCCC CTGTTTGCTC CTCCGATAAC | 750 |
| TGGGGTGACC TTGGTTAATA TTCACCAGCA GCCTCCCCCG TTGCCCCTCT | 800 |
| GGATCCACTG CTTAAATACG GACGAGGACA GGGCCCTGTC TCCTCAGCTT | 850 |
| CAGGCACCAC CACTGACCTG GGACAGTCCT AGGTGCTTGT TCTTTTTGCA | 900 |
| GAAGCTCAGA ATAAACGCTC AACTTTGGCA GATACTAGTC AGGTAAGTAT | 950 |
| CAAGGTTACA AGACAGGTTT AAGGAGACCA ATAGAAACTG GGCTTGTCGA | 1000 |
| GACAGAGAAG ACTCTTGCGT TTCTGATAGG CACCTATTGG TCTTACTGAC | 1050 |
| 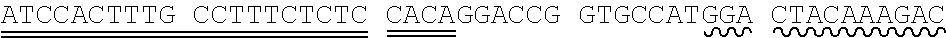 | 1100 |
| 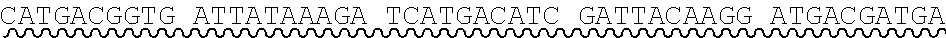 | 1150 |
| 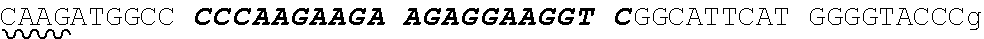 | 1200 |
| ccgctatggc tgagaggccc ttccagtgtc gaatctgcat gcagaacttc | 1250 |
| agtcagtccg gcaacctggc ccgccacatc cgcacccaca ccggcgagaa | 1300 |
| gccttttgcc tgtgacattt gtgggaggaa atttgccctg aagcagaacc | 1350 |
| tgtgtatgca taccaagata cacacgggcg agaagccctt ccagtgtcga | 1400 |
| atctgcatgc agaagtttgc ctggcagtcc aacctgcaga accataccaa | 1450 |
| gatacacacg ggcgagaagc ccttccagtg tcgaatctgc atgcgtaact | 1500 |
| tcagtacctc cggcaacctg acccgccaca tccgcaccca caccggcgag | 1550 |
| aagccttttg cctgtgacat ttgtgggagg aaatttgccc gccgctccca | 1600 |
| 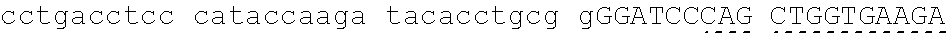 | 1650 |
| 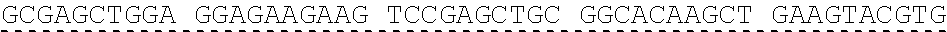 | 1700 |
| 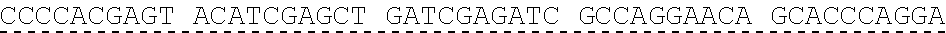 | 1750 |
| 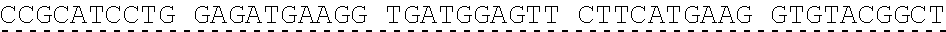 | 1800 |
| 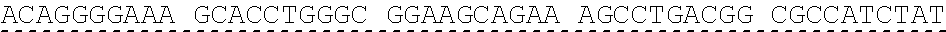 | 1850 |
| 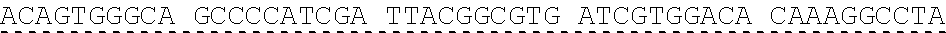 | 1900 |
| 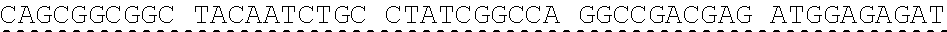 | 1950 |
| 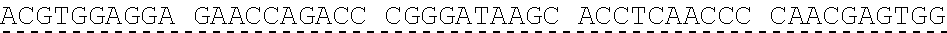 | 2000 |
| 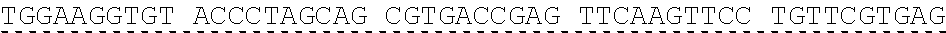 | 2050 |
| 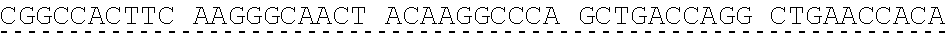 | 2100 |
| 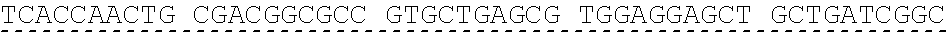 | 2150 |
| 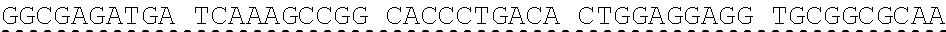 | 2200 |
| 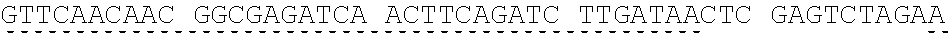 | 2250 |
| 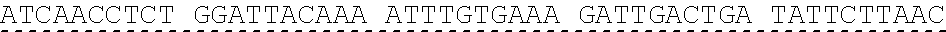 | 2300 |
| 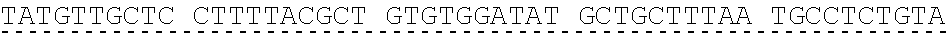 | 2350 |
| 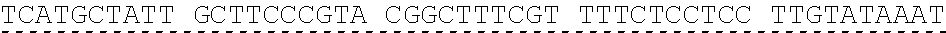 | 2400 |
| 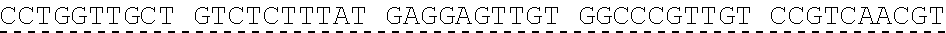 | 2450 |
| 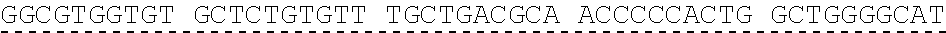 | 2500 |
| 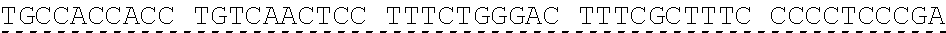 | 2550 |
| 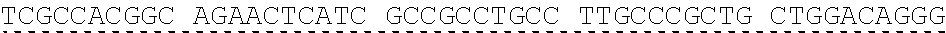 | 2600 |
| 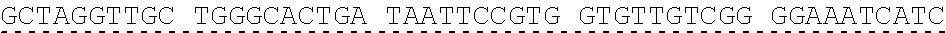 | 2650 |
| 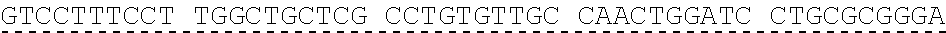 | 2700 |
| 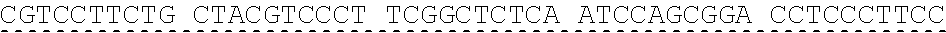 | 2750 |
| 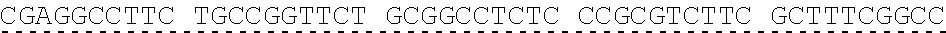 | 2800 |
| 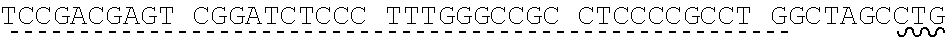 | 2850 |
| 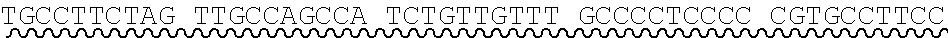 | 2900 |
| 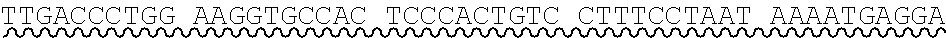 | 2950 |
| 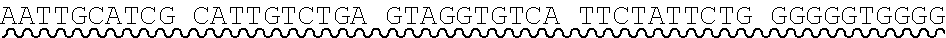 | 3000 |
| 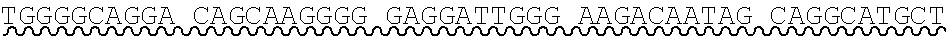 | 3050 |
| 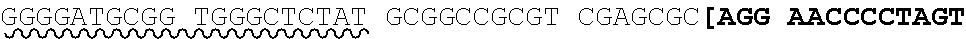 | 3100 |
| GATGGAGTTG GCCACTCCCT CTCTGCGCGC TCGCTCGCTC ACTGAGGCCG | 3150 |
| CCCGGGCTTT GCCCGGGCGG CCTCAGTGAG CGAGCGAGCG CGCAG | 3195 |

The complete nucleotide sequence for the SB-71728 AAV2/6 vector is shown below. The specific annotations shown in Table 5 are indicated in the sequence text as shown in Table 5.

TABLE 5
Elements of SB71728 AAV (SEQ ID NO: 31)

| Nucleotide | | SEQ | |
|---|---|---|---|
| Position- | Feature/ | ID | |
| annotation | Desription | NO: | Sequence |
| 1-130 | 5′ ITR | 1 | CTGCGCGCTCGCTCGCTCACTGAGGCCGCCCGGGCAAAGCCCGGGCGTCG |
| | [plain text in | | GGCGACCTTTGGTCGCCCGGCCTCAGTGAGCGAGCGAGCGCGCAGAGAGG |
| | brackets] | | GAGTGGCCAACTCCATCACTAGGGGTTCCT |
| 156-476 | ApoE | 32 | AGGCTCAGAGGCACACAGGAGTTTCTGGGCTCACCCTGCCCCCTTCCAAC |
| | (Enhancer) | | CCCTCAGTTCCCATCCTCCAGCAGCTGTTTGTGTGCTGCCTCTGAAGTCC |
| | underlined | | ACACTGAACAAACTTCAGCCTACTCATGTCCCTAAAATGGGCAAACATTG |
| | | | CAAGCAGCAAACAGCAAACACACAGCCCTCCCTGCCTGCTGACCTTGGAG |
| | | | CTGGGGCAGAGGTCAGAGACCTCTCTGGGCCCATGCCACCTCCAACATCC |
| | | | ACTCGACCCCTTGGAATTTCGGTGGAGAGGAGCAGAGGTTGTCCTGGCGT |
| | | | GGTTTAGGTAGTGTGAGAGGG |
| 485-877 | hAAT | 33 | GATCTTGCTACCAGTGGAACAGCCACTAAGGATTCTGCAGTGAGAGCAGA |
| | (Promoter) | | GGGCCAGCTAAGTGGTACTCTCCCAGAGACTGTCTGACTCACGCCACCCC |
| | italics | | CTCCACCTTGGACACAGGACGCTGTGGTTTCTGAGCCAGGTACAATGACT |
| | | | CCTTTCGGTAAGTGCAGTGGAAGCTGTACACTGCCCAGGCAAAGCGTCCG |
| | | | GGCAGCGTAGGCGGGCGACTCAGATCCCAGCCAGTGGACTTAGCCCCTGT |
| | | | TTGCTCCTCCGATAACTGGGGTGACCTTGGTTAATATTCACCAGCAGCCT |
| | | | CCCCCGTTGCCCCTCTGGATCCACTGCTTAAATACGGACGAGGACAGGGC |
| | | | CCTGTCTCCTCAGCTTCAGGCACCACCACTGACCTGGGACAGT |
| 886-933 | 5′ UTR | 18 | CTTGTTCTTTTTGCAGAAGCTCAGAATAAACGCTCAACTTTGGCAGAT |
| | Bold | | |
| 943-1075 | Human β | 3 | GTAAGTATCAAGGTTACAAGACAGGTTTAAGGAGACCAATAGAAACTGGG |
| | globin/IgG | | CTTGTCGAGACAGAGAAGACTCTTGCGTTTCTGATAGGCACCTATTGGTC |
| | chimeric | | TTACTGACATCCACTTTGCCTTTCTCTCCACAG |
| | intron | | |
| | (Intron) | | |
| | double | | |
| | underlined | | |
| 1086-1154 | N-terminal | 34 | GACTACAAAGACCATGACGGTGATTATAAAGATCATGACATCGATTACAA |
| | peptide | | GGATGACGATGACAAG |
| 1161-1181 | Nuclear | 36 | CCCAAGAAGAAGAGGAAGGTC |
| | localization | | |
| | signal | | |
| | Bold italic | | |
| 1200-1715 | ZFP 71728 | 29 | GCCGCTATGGCTGAGAGGCCCTTCCAGTGTCGAATCTGCATGCGTAACTT |
| | DNA- | | CAGTCAGTCCTCCGACCTGTCCCGCCACATCCGCACCCACACCGGCGAGA |
| | binding | | AGCCTTTTGCCTGTGACATTTGTGGGAGGAAATTTGCCCTGAAGCACAAC |
| | domain | | CTGCTGACCCATACCAAGATACACACGGGCGAGAAGCCCTTCCAGTGTCG |
| | 1ower case | | AATCTGCATGCAGAACTTCAGTGACCAGTCCAACCTGCGCGCCCACATCC |
| | | | GCACCCACACCGGCGAGAAGCCTTTTGCCTGTGACATTTGTGGGAGGAAA |
| | | | TTTGCCCGCAACTTCTCCCTGACCATGCATACCAAGATACACACCGGAGA |
| | | | GCGCGGCTTCCAGTGTCGAATCTGCATGCGTAACTTCAGTCTGCGCCACG |
| | | | ACCTGGAGCGCCACATCCGCACCCACACCGGCGAGAAGCCTTTTGCCTGT |
| | | | GACATTTGTGGGAGGAAATTTGCCCACCGCTCCAACCTGAACAAGCATAC |
| | | | CAAGATACACCTGCGG |
| 1722-2315 | FokI-KKR | 39 | CAGCTGGTGAAGAGCGAGCTGGAGGAGAAGAAGTCCGAGCTGCGGCACAA |
| | nuclease | | GCTGAAGTACGTGCCCCACGAGTACATCGAGCTGATCGAGATCGCCAGGA |
| | domain | | ACAGCACCCAGGACCGCATCCTGGAGATGAAGGTGATGGAGTTCTTCATG |
| | P478S | | AAGGTGTACGGCTACAGGGGAAAGCACCTGGGCGGAAGCAGAAAGCCTGA |
| | Dashed | | CGGCGCCATCTATACAGTGGGCAGCCCCATCGATTACGGCGTGATCGTGG |
| | underlined | | ACACAAAGGCCTACAGCGGCGGCTACAATCTGAGCATCGGCCAGGCCGAC |
| | | | GAGATGCAGAGATACGTGAAGGAGAACCAGACCCGGAATAAGCACATCAA |
| | | | CCCCAACGAGTGGTGGAAGGTGTACCCTAGCAGCGTGACCGAGTTCAAGT |
| | | | TCCTGTTCGTGAGCGGCCACTTCAAGGGCAACTACAAGGCCCAGCTGACC |
| | | | AGGCTGAACCGCAAAACCAACTGCAATGGCGCCGTGCTGAGCGTGGAGGA |
| | | | GCTGCTGATCGGCGGCGAGATGATCAAAGCCGGCACCCTGACACTGGAGG |
| | | | AGGTGCGGCGCAAGTTCAACAACGGCGAGATCAACTTCTGATAA |
| 2328-2919 | WPREmut6 | 37 | AATCAACCTCTGGATTACAAAATTTGTGAAAGATTGACTGATATTCTTAA |
| | 3′UTR | | CTATGTTGCTCCTTTTACGCTGTGTGGATATGCTGCTTTAATGCCTCTGT |
| | Dotted | | ATCATGCTATTGCTTCCCGTACGGCTTTCGTTTTCTCCTCCTTGTATAAA |
| | underlined | | TCCTGGTTGCTGTCTCTTTATGAGGAGTTGTGGCCCGTTGTCCGTCAACG |
| | | | TGGCGTGGTGTGCTCTGTGTTTGCTGACGCAACCCCCACTGGCTGGGGCA |
| | | | TTGCCACCACCTGTCAACTCCTTTCTGGGACTTTCGCTTTCCCCCTCCCG |
| | | | ATCGCCACGGCAGAACTCATCGCCGCCTGCCTTGCCCGCTGCTGGACAGG |
| | | | GGCTAGGTTGCTGGGCACTGATAATTCCGTGGTGTTGTCGGGGAAATCAT |
| | | | CGTCCTTTCCTTGGCTGCTCGCCTGTGTTGCCAACTGGATCCTGCGCGGG |
| | | | ACGTCCTTCTGCTACGTCCCTTCGGCTCTCAATCCAGCGGACCTCCCTTC |
| | | | CCGAGGCCTTCTGCCGGTTCTGCGGCCTCTCCCGCGTCTTCGCTTTCGGC |
| | | | CTCCGACGAGTCGGATCTCCCTTTGGGCCGCCTCCCCGCCTG |
| 2926-3148 | Poly- | 7 | CTGTGCCTTCTAGTTGCCAGCCATCTGTTGTTTGCCCCTCCCCCGTGCCT |
| | adenylation | | TCCTTGACCCTGGAAGGTGCCACTCCCACTGTCCTTTCCTAATAAAATGA |
| | signal | | GGAAATTGCATCGCATTGTCTGAGTAGGTGTCATTCTATTCTGGGGGGTG |
| | | | GGGTGGGGCAGGACAGCAAGGGGGAGGATTGGGAAGACAATAGCAGGCAT |
| | | | GCTGGGGATGCGGTGGGCTCTAT |
| 3166-3273 | 3′ ITR | 8 | AGGAACCCCTAGTGATGGAGTTGGCCACTCCCTCTCTGCGCGCTCGCTCG |
| | [Bold text | | CTCACTGAGGCCGCCCGGGCTTTGCCCGGGCGGCCTCAGTGAGCGAGCGA |
| | in brackets] | | GCGCGCAG |

### Complete Sequence of 71728 AAV:

(SEQ ID NO: 31)

| | |
|---|---|
| [CTGCGCGCTC GCTCGCTCAC TGAGGCCGCC CGGGCAAAGC CCGGGCGTCG | 50 |
| GGCGACCTTT GGTCGCCCGG CCTCAGTGAG CGAGCGAGCG CGCAGAGAGG | 100 |
| GAGTGGCCAA CTCCATCACT AGGGGTTCCT] GCGGCCTAAG CTTGAGCTCT | 150 |
| TCGAAAGGCT CAGAGGCACA CAGGAGTTTC TGGGCTCACC CTGCCCCCTT | 200 |
| CCAACCCCTC AGTTCCCATC CTCCAGCAGC TGTTTGTGTG CTGCCTCTGA | 250 |
| AGTCCACACT GAACAAACTT CAGCCTACTC ATGTCCCTAA AATGGGCAAA | 300 |
| CATTGCAAGC AGCAAACAGC AAACACACAG CCCTCCCTGC CTGCTGACCT | 350 |
| TGGAGCTGGG GCAGAGGTCA GAGACCTCTC TGGGCCCATG CCACCTCCAA | 400 |
| CATCCACTCG ACCCCTTGGA ATTTCGGTGG AGAGGAGCAG AGGTTGTCCT | 450 |
| GGCGTGGTTT AGGTAGTGTG AGAGGGGTCC CGGGGATCTT GCTACCAGTG | 500 |
| GAACAGCCAC TAAGGATTCT GCAGTGAGAG CAGAGGGCCA GCTAAGTGGT | 550 |
| ACTCTCCCAG AGACTGTCTG ACTCACGCCA CCCCCTCCAC CTTGGACACA | 600 |
| GGACGCTGTG GTTTCTGAGC CAGGTACAAT GACTCCTTTC GGTAAGTGCA | 650 |
| GTGGAAGCTG TACACTGCCC AGGCAAAGCG TCCGGGCAGC GTAGGCGGGC | 700 |
| GACTCAGATC CCAGCCAGTG GACTTAGCCC CTGTTTGCTC CTCCGATAAC | 750 |
| TGGGGTGACC TTGGTTAATA TTCACCAGCA GCCTCCCCCG TTGCCCCTCT | 800 |
| GGATCCACTG CTTAAATACG GACGAGGACA GGGCCCTGTC TCCTCAGCTT | 850 |
| CAGGCACCAC CACTGACCTG GGACAGTCCT AGGTGCTTGT TCTTTTTGCA | 900 |
| GAAGCTCAGA ATAAACGCTC AACTTTGGCA GATACTAGTC AGGTAAGTAT | 950 |
| CAAGGTTACA AGACAGGTTT AAGGAGACCA ATAGAAACTG GGCTTGTCGA | 1000 |
| GACAGAGAAG ACTCTTGCGT TTCTGATAGG CACCTATTGG TCTTACTGAC | 1050 |
| 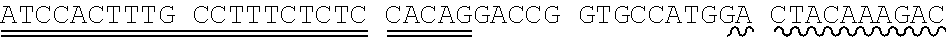 | 1100 |
| 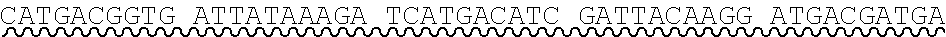 | 1150 |
| 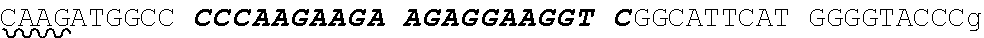 | 1200 |
| ccgctatggc tgagaggccc ttccagtgtc gaatctgcat gcgtaacttc | 1250 |
| agtcagtcct ccgacctgtc ccgccacatc cgcacccaca ccggcgagaa | 1300 |
| gccttttgcc tgtgacattt gtgggaggaa atttgccctg aagcacaacc | 1350 |
| tgctgaccca taccaagata cacacgggcg agaagccctt ccagtgtcga | 1400 |
| atctgcatgc agaacttcag tgaccagtcc aacctgcgcg cccacatccg | 1450 |
| cacccacacc ggcgagaagc cttttgcctg tgacatttgt gggaggaaat | 1500 |
| ttgcccgcaa cttctccctg accatgcata ccaagataca caccggagag | 1550 |
| cgcggcttcc agtgtcgaat ctgcatgcgt aacttcagtc tgcgccacga | 1600 |
| cctggagcgc cacatccgca cccacaccgg cgagaagcct tttgcctgtg | 1650 |
| acatttgtgg gaggaaattt gcccaccgct ccaacctgaa caagcatacc | 1700 |
| 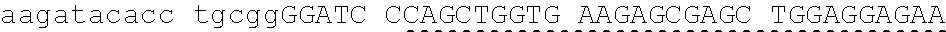 | 1750 |
| 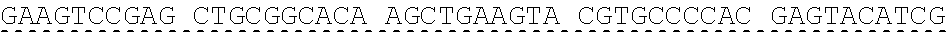 | 1800 |
| 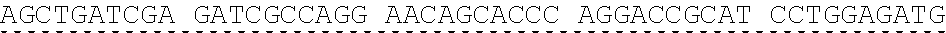 | 1850 |
| 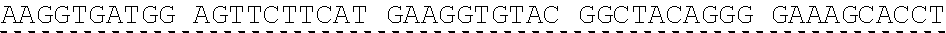 | 1900 |
| 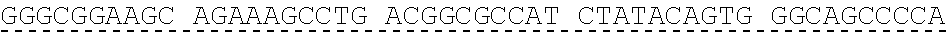 | 1950 |
| 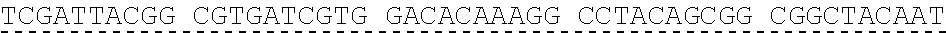 | 2000 |
| 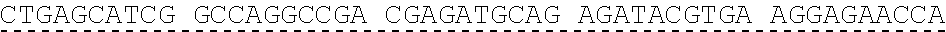 | 2050 |
| 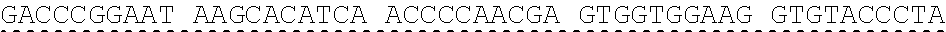 | 2100 |
| 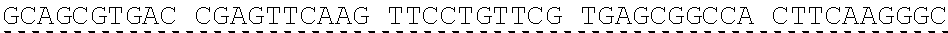 | 2150 |
| 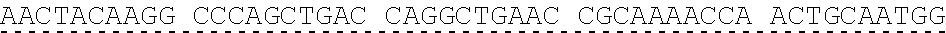 | 2200 |
| 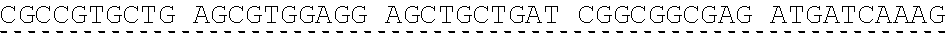 | 2250 |
| 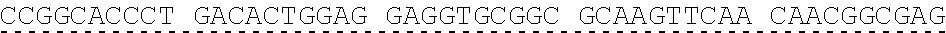 | 2300 |
| 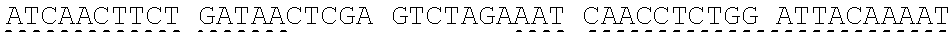 | 2350 |
| 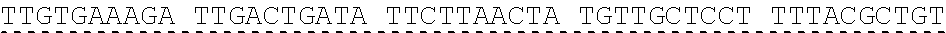 | 2400 |
| 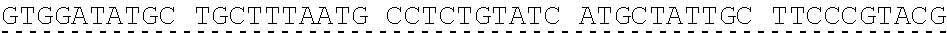 | 2450 |
| 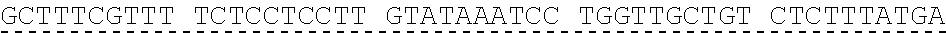 | 2500 |
| 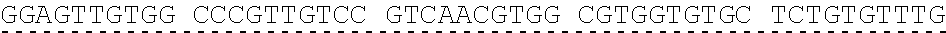 | 2550 |
| 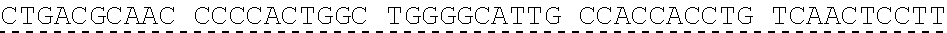 | 2600 |
| 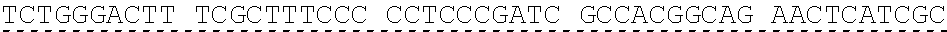 | 2650 |
| 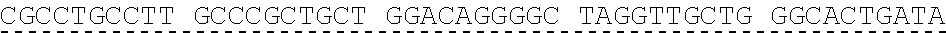 | 2700 |
| 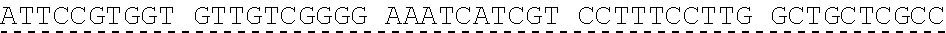 | 2750 |
| 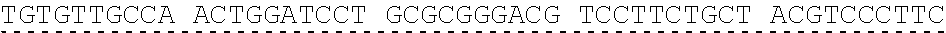 | 2800 |
| 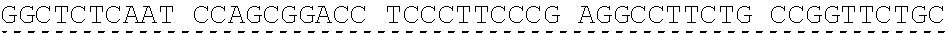 | 2850 |
| 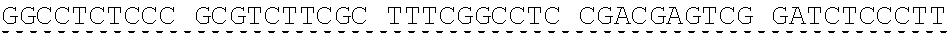 | 2900 |
| 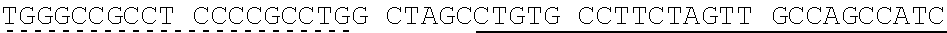 | 2950 |
| 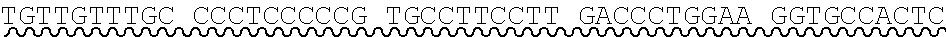 | 3000 |
| 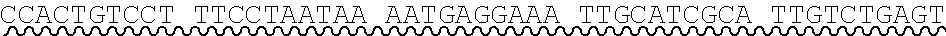 | 3050 |
| 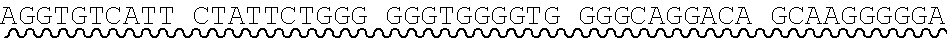 | 3100 |
| 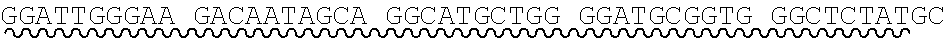 | 3150 |
| GGCCGCGTCG AGCGC[AGGAA CCCCTAGTGA TGGAGTTGGC CACTCCCTCT | 3200 |
| CTGCGCGCTC GCTCGCTCAC TGAGGCCGCC CGGGCTTTGC CCGGGCGGCC | 3250 |
| TCAGTGAGCG AGCGAGCGCG CAG] | 3273 |

### Example 2

Compositions comprising the polynucleotides and AAVs as described in Example 1 were prepared as follows: The components were supplied in three capped vials: one for ZFN1 (SB-47171, white capped and labeled SB-A6P-ZLEFT); ZFN2 (SB47898, blue capped and labeled SB-A6P-ZRIGHT); and hIDS Donor (hIDS, orange capped and labeled SB-A6P-HNT). The product components were all purified AAV individually formulated in phosphate buffered saline (PBS) containing CaCl2, MgCl2, NaCl, sucrose and Kolliphor® (Poloxamer) P188 or in a Normal Saline (NS) formulation. Dose calculations were performed using the subject's weight and rounded to two decimal points. The calculations were done by multiplying the cohort dose by the subject weight at baseline, and then dividing by the vg/mL concentration. The three product component volumes were added together and the total volume determined. In addition, the volume of human serum albumin (HSA) intravenous solution for addition was calculated to achieve a final concentration of 0.25% HSA and finally the PBS or NS was added the required amount to achieve the correct component concentration.

The product components were then added to an IV infusion bag containing 0.25% HSA in NS or PBS. Each product component was added separately and then the bag was mixed gently and transferred to the person responsible for infusion. The product was then infused into subjects at a rate of 100 mL/hour using an infusion pump (Sigma Spectrum).

### Example 3

Study Eligibility and Exclusion Criteria

Key eligibility criteria for subjects in the study included: ≥5 years of age (Adult cohorts 1 through 3: ≥18 years of age; Pediatric cohorts 4 and 6: 12 to 17 years of age; Pediatric cohorts 5 and 7: 5 to 11 years of age); clinical diagnosis of MPS II based on evidence of hepatosplenomegaly, dysostosis multiplex by X-ray, valvular heart disease and/or obstructive airway disease with IDS deficiency confirmed by gene sequencing; Magnetic resonance imaging (MRI) negative for liver mass.

Key exclusion criteria for subjects in the study included: known unresponsiveness to enzyme replacement therapy; neutralizing antibodies in the serum to AAV6; receiving anti-retroviral therapy for hepatitis B or C, or active hepatitis B or hepatitis C or human immunodeficiency virus (HIV) 1/2; lack of tolerance to idursulfase treatment (Elaprase®); polymorphisms in the ZFN targeted region in the albumin locus; liver fibrosis score of 3 or 4 on a 0 to 4 point scale (Desmet et al. (1994) Hepatology 19(6):1513-20), markers of hepatic dysfunction; pregnant or breastfeeding female; contraindication to the use of corticosteroids; current treatment with systemic immunomodulatory agent or steroid use; prior treatment with a gene therapy product; and elevated or abnormal α-fetoprotein.

Study Design

The study was performed on subjects with MPS II disease. Several cohorts were studied: i) age≥18 years (adult cohorts 1 through 3); ii) age 12-17 years (pediatric cohorts 4 and 6); and iii) age 5-11 (pediatric cohorts 5 and 7). The doses used in these cohorts are shown below in Table 6. Cohort 1 is considered the low dose, cohort 2 is the mid dose, and cohort 3 is the high dose. For all cohorts, total AAV dose includes 2 ZFN vectors and 1 donor vector in a fixed ratio of 1:1:8.

TABLE 6
Evaluation doses

| | ZFN 1 | ZEN 2 | hIDS | | |
|---|---|---|---|---|---|
| | (SB- | (SB- | donor (SB- | Total | |
| | 41717) | 47898) | IDS) | rAAV | |
| Cohort | vg/kg | vg/kg | vg/kg | vg/kg | |
| 1 | 5.00e+11 | 5.00e+11 | 4.00e+12 | 5.00e+12 | starting |
| | | | | | dose |
| 2, 4, 5 | 1.00e+12 | 1.00e+12 | 8.00e+12 | 1.00e+13 | 2X starting |
| | | | | | dose |
| 3, 6, 7 | 5.00e+12 | 5.00e+12 | 4.00e+13 | 5.00e+13 | 10x starting |
| | | | | | dose |

Clinical Endpoints

Primary endpoint: The primary endpoint of this study were the safety and tolerability of the composition as assessed by incidence of adverse events and significant adverse events. Additional safety evaluations included: routine hematology, chemistry, and liver function laboratory tests, vital signs, physical exam, ECG, ECHO, and concomitant medications; cranial nerve exam and muscle strength testing; serial α-fetoprotein testing and MM of liver to evaluate for liver mass. Safety assessment was performed on all subjects. All reported adverse events were coded to a standard set of terms using the Medical Dictionary for Regulatory Activities (MedDRA) AE dictionary. The frequency of each event was summarized by severity and by relatedness to the study drug.

Key secondary endpoints included: change from baseline in: IDS activity measured in plasma, total GAG, DS GAG, and HS GAG levels (expressed as a ratio to creatinine) measured in urine; AAV2/6 clearance measured by vector genomes in plasma, saliva, urine, stool, and semen by PCR. Urine GAG levels are a key biomarker of MPS II disease pathophysiology. Additional secondary endpoints include monitoring monthly frequency and dose of IDS (or equivalent ERT).

Key exploratory endpoints included a change from baseline in: percentage and durability of gene modification at the albumin locus in liver tissue obtained at biopsy; forced vital capacity measured by PFTs; distance walked measured by 6MWT; JROM; MRI of liver to evaluate liver and spleen volume; MRI of brain and cervical spine to evaluate clinical soft tissue and/or bone; neurocognitive abilities by WASI-II, WPPSI-IV, or BSID-III, and by VABS-II; histopathological exam of liver tissue; total GAG, DS GAG, and HS GAG levels measured in liver tissue and CSF and immune response to AAV 2/6 and ZFNs measured in serum. The analyses of these endpoints was descriptive and exploratory in nature. Continuous variables were summarized by means, standard deviations, medians, and ranges for all enrolled subjects. Categorical variables were summarized with counts and percentages per category for all enrolled subjects. Change from the preceding year values may be calculated for selected clinical and laboratory parameters. Shift-tables (Change-from-Baseline relative to the normal range) may be constructed for selected laboratory parameters.

Statistical Analysis and Data Analysis

This was an exploratory Phase I study and thus there will be limited statistical power to evaluate efficacy and related biological endpoints. Therefore, analyses were primarily descriptive and exploratory in nature. This study will enroll up to 23 subjects (2 subjects in each of 7 cohorts, with potential enrollment of 3 additional subjects at the maximal tolerated dose in each of 3 age cohorts). The selection of 2 subjects per cohort was not based on statistical calculations since this is a Phase I safety study to evaluate safety and tolerability. All tables, listings, and data summaries were performed in SAS version 9.2 or later.

Patients

The patient demographics are shown below in Table 7, which demonstrates that all patients had attenuated MPS II disease. Table 8 lists the exposure to treatment that each subject had at 32 weeks post trial initiation.

TABLE 7
Patient Demographics

| | |
|---|---|
| Subject Characteristics | Overall (N = 8) |

Age (Years)

| | |
|---|---|
| number of patients | 8 |
| Min-Max | 19-61 |
| Mean (SD) | 35.58 (15.59) |

Sex, n (%)

| | |
|---|---|
| Male | 8 (100%) |

Race, n (%)

| | |
|---|---|
| Asian | 1 (12.5%) |
| White | 7 (87.5%) |

TABLE 8
Treatment exposure (approximate)

| Subject | Dose Cohort | Follow-Up (Weeks) |
|---|---|---|
| 1 | 1 | 57 |
| 2 | 1 | 49 |
| 3 | 2 | 39 |
| 4 | 2 | 34 |
| 5 | 3 | 25 |
| 6 | 3 | 19 |
| 7 | 3 | 2 |
| 8 | 3 | 1 |

Observed Adverse Events

All subjects reported treatment emergent adverse events (TEAEs), consistent with ongoing MPS II disease. Most were mild (gradel) and resolved without treatment. Three Serious Adverse Events (SAEs) were reported in three subjects and were not considered related to the study drug by the site investigator. These were A) Grade 3 bronchitis, which was thought to be secondary to the subject's medical history of chronic pulmonary disease from their MPS II, wherein the bronchitis was resolved after medical treatment; B) Grade 2 atrial fibrillation. This was thought to be secondary to the subject's medical history of cardiac valve disease from their MPS II disease, and the event resolved after medical treatment; and C) Umbilical Hernia, obstructive, thought to be secondary to subject's underlying MPS II disease, medical history of hernias and morbid obesity. These events are summarized in Table 9A below:

TABLE 9A
Serious Adverse Events

| | | Study | | | | |
|---|---|---|---|---|---|---|
| Event | Group | Day | Grade | Outcome | Related | Comments |
| Bronchitis | 1 | 20 | 3 | Resolved | Not related | Secondary to |
| | (5e12vg/kg) | | | | | subject's medical |
| | | | | | | history of chronic |
| | | | | | | pulmonary |
| | | | | | | disease from MPS |
| | | | | | | II |
| Atrial | 2 | 52 | 2 | Resolved | Not related | Secondary to |
| fibrillation | (1e13vg/kg) | | | | | subject's medical |
| | | | | | | history of cardiac |
| | | | | | | valve disease |
| | | | | | | from MPS II |
| Umbilical | 3 | 121 | 3 | Resolved | Not related | Secondary to |
| hernia, | (5e13vg/kg) | | | | | subject's |
| obstructive | | | | | | underlying MPS II |
| | | | | | | disease, medical |
| | | | | | | history of hernias, |
| | | | | | | and morbid |
| | | | | | | obesity |

All but two study drug-related Adverse Events (AEs) were mild (Grade 1), and all resolved without intervention and were not dose-dependent. The AEs are shown below in Table 10.

TABLE 10
Study Drug-related Adverse Events

| | Cohort 1 | Cohort 2 | Cohort 3 | Overall |
|---|---|---|---|---|
| | (N = 2) | (N = 2) | (N = 4) | (N = 8) |
| Preferred Term | n [T] | n [T] | n [T] | n [T] |
| Any Event | 2 [5] | 1 [5] | 2 [7] | 5 [17] |
| 1—Mild | 2 [5] | 1 [5] | 2 [5] | 5 [15] |
| 2—Moderate | none | none | 1 [2] | 1 [2] |
| ALT increased | 0 [0] | 1 [1] | 0 [0] | 1 [1] |
| AST increased | 0 [0] | 1 [1] | 0 [0] | 1 [1] |
| Asthenia | 1 [1] | 0 [0] | 0 [0] | 1 [1] |
| Cold sweat | 1 [1] | 0 [0] | 0 [0] | 1 [1] |
| Dizziness | 1 [1] | 0 [0] | 0 [0] | 1 [1] |
| Erythema | 0 [0] | 1 [2] | 0 [0] | 1 [2] |
| Flushing | 0 [0] | 1 [1] | 1 [2] | 1 [2] |
| Pruritus | 1 [2] | 0 [0] | 1 [1] | 2 [3] |
| Dysgeusia | 0 [0] | 0 [0] | 1 [1] | 1 [1] |
| Headache | 0 [0] | 0 [0] | 1 [1]* | 1 [1] |
| Pyrexia | 0 [0] | 0 [0] | 1 [1]* | 1 [1] |
| Transaminases | 0 [0] | 0 [0] | 1 [2] | 1 [2] |
| increased | | | | |

In Table 10, ‘N’ indicates the total number of subjects in each treatment group; ‘n’ indicates the number of subjects with an adverse event for each preferred term; and ‘[T]’ indicates the total number of adverse events. * indicates moderate severity (Grade2).

### Preliminary Liver Genome Editing Results

An RT-qPCR assay was used to detect a unique mRNA that occurs in liver tissue when the IDS transgene is inserted into the albumin gene (see Example 4). Results were positive (+) in both Cohort 2 subjects who received the 1e13 vg/kg dose (see Table 11 below). A less sensitive genomic DNA assay to detect insertions/deletions at the target site in the albumin gene was negative in all samples tested.

TABLE 11
Albumin-IDS mRNA assay

| | Week 24 Results | | | | | |
|---|---|---|---|---|---|---|
| | Cohort 1 | | Cohort 2 | | Cohort 3 | |
| Subject | 1 | 2* | 3 | 4 | 5* | 6 |
| Integration Assay | − | n/a | + | + | n/a | pending |

*no results available as liver biopsy procedure contraindicated

Preliminary Plasma IDS Measurements

Plasma IDS activity was measured at trough, which was defined as in the period immediately prior to ERT dosing when possible, and no less than 96 hours after the subject's last ERT infusion. IDS was measured in this study using a standard validated fluorometric assay using 4-methylumbelliferyl sulfate (4MU) as substrate (see below in Example 4 for exemplary plasma IDS assays). Plasma IDS activity was below the level of quantification (5.2 nmol/hr/mL) at the baseline trough, and for the first 16 weeks post-dosing.

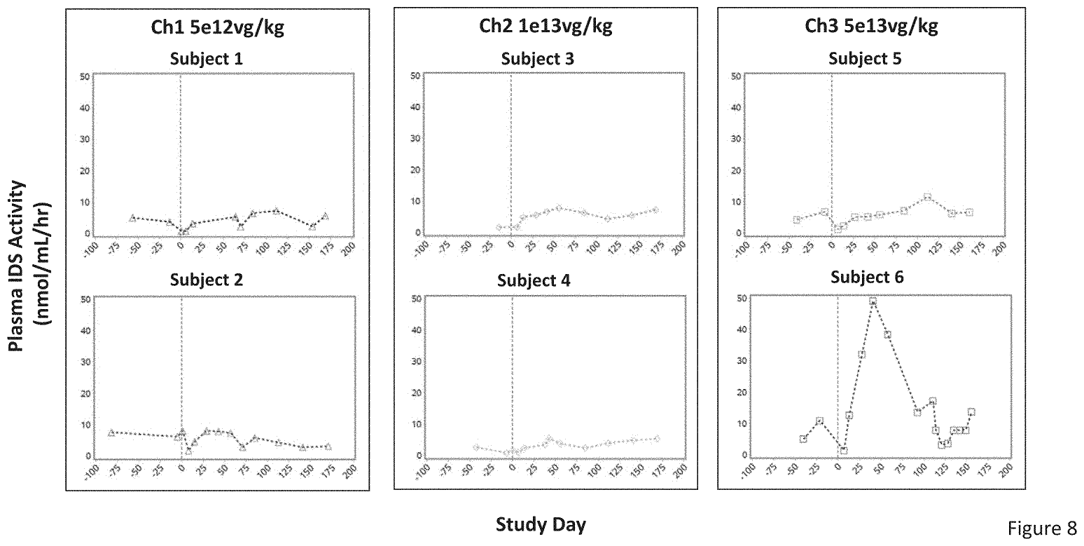

An improved plasma IDS activity assay was developed (see Example 4). As shown in FIG. 8, through use of the new assay, plasma IDS was detectable in all subjects. As above, samples obtained less than 96 hours post-ERT dosing were excluded. In this assay, MPS II baseline subjects are <10 mmol/mL/hr IDS activity, with a baseline in a normal population being >79 mmol/mL/hr. FIG. 8 shows the plasma IDS levels in subjects prior to treatment with the study drug (see Study Day data labeled as negative prior to dosing on day 0).

12-Week Urine GAG Results, Cohorts 1 and 2

At twelve weeks post-dosing with the compositions comprising the polynucleotides and AAVs as described in Example 1, there was a decrease in all GAG biochemical markers in cohort 2. Total urine GAG levels were measured by validated 1,9-dimethylene blue (DMB) colorimetric assay (see Example 4) while urine dermatan sulfate and urine heparan sulfate GAG levels were measured by a validated MS/MS assay (ultra-performance liquid chromatography followed by tandem-mass spectrometry). Exemplary MS/MS assay procedures are described in Example 4. Reductions in Cohort 2 in total GAGs, and in dermatan sulfate and heparan sulfate, the two GAGs most closely linked to MPS II, were observed. These results are shown below in Table 8A.

TABLE 8A
12 week urine GAG results:

| | Total GAG | Dermatan Sulfate | Heparan Sulfate |
|---|---|---|---|
| | % Change at | % Change at | % Change at |
| | 12 weeks | 12 weeks | 12 weeks |
| | Mean (SD) | Mean (SD) | Mean (SD) |
| Cohort 1 (Subject 1) | +32.7 | +16.1 | +26.1 |
| Cohort 1 (Subject 2) | −14.3 | +28.2 | −7.1 |
| Cohort 1 Mean (SD) | +9.2 (33.2) | +22.2 (8.5) | +9.5 (23.5) |
| Cohort 2 (Subject 3) | −42.7 | −28.6 | −61.0 |
| Cohort 2 (Subject 4) | −20.2 | −10.9 | −46.0 |
| Cohort 2 Mean (SD) | −31.4 (15.9) | −19.7 (12.6) | −53.5 (10.6) |

16-Week Urine GAG Results, Cohorts 1 and 2

At sixteen weeks post-dosing with the compositions comprising the polynucleotides and AAVs as described in Example 1, there was a decrease in all GAG biochemical markers in cohort 2. Total urine GAG levels were measured by validated 1,9-dimethylene blue (DMB) colorimetric assay (see Example 4) while urine dermatan sulfate and urine heparan sulfate GAG levels were measured by a validated MS/MS assay (ultra-performance liquid chromatography followed by tandem-mass spectrometry). Exemplary MS/MS assay procedures are described in Example 4. These results are shown below in Table 8B.

TABLE 8B
16-week Urine Analysis

| | Total GAG | Dermatan Sulfate | Heparan Sulfate |
|---|---|---|---|
| | % Change at | % Change at | % Change at |
| | 16 weeks | 16 weeks | 16 weeks |
| | Mean (SD) | Mean (SD) | Mean (SD) |
| Cohort 1 (Subject 1) | +13.0 | −14.5 | −15.6 |
| Cohort 1 (Subject 2) | +4.8 | +22.6 | −31.4 |
| Cohort 1 Mean (SD) | +8.9 (5.8) | +4.1 (26.2) | −23.5 (11.2) |
| Cohort 2 (Subject 3) | −62.5 | −47.4 | −69.9 |
| Cohort 2 (Subject 4) | −39.1 | −16.3 | −53.0 |
| Cohort 2 Mean (SD) | −50.8 (16.5) | −31.8 (22.0) | −61.5 (12.0) |

Summary of Results at 16 Weeks

Enrollment and dosing for cohorts 1, 2, and 3 was completed, although 16 week data was only available at this preliminary report for cohorts 1 and 2. Therefore, current data are only from the low dose and mid dose. The two patients from cohort 3 were recently treated at a dose of 5e13 vg/kg which is 5 times higher than the cohort 2 dose. The compositions comprising the polynucleotides and AAVs as described in Example 1 were administered to six subjects with attenuated MPS II at a dose of up to 5.00e13 vg/kg and was generally well tolerated in all subjects. No SAEs related to the composition were reported and no persistent transaminitis was observed. Urine GAG biomarkers showed a reduction in total GAGs, dermatan sulfate (DS GAG) and heparan sulfate (HS GAG) in a dose dependent manner. Two subjects treated at the 1.00e13 vg/kg dose (cohort 2) showed a mean decrease of greater than 60% in urine heparan sulfate at 16 weeks post dosing with the compositions described herein. A mean reduction in urine heparan sulfate of approximately 61.5% for cohort 2, including an approximate 53% for the first patient and approximately 69.9% for the second patient, was observed. For dermatan sulfate, a mean reduction of approximately 31.8 for cohort 2, including an approximate 47.4% for the first patient and approximately 16.3% for the second patient, was observed. A mean reduction in urine heparan sulfate of approximately 50.8% for cohort 2, including an, and of approximately 62.5% for the first patient and approximately 39.1% for the second patient, was observed. For Cohort 2 patients, total urinary GAGs, dermatan sulfate and heparan sulfate each declined below baseline for both patients. Specifically, total urinary GAGs declined by 51%, dermatan sulfate by 32%, and heparan sulfate by 62% in Cohort 2 (mid dose) at 16 weeks.

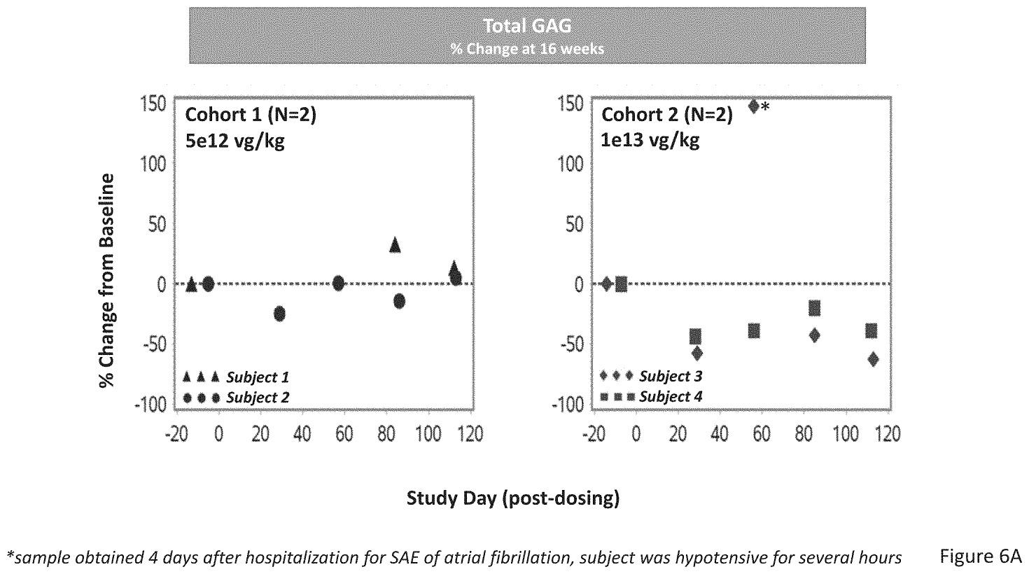

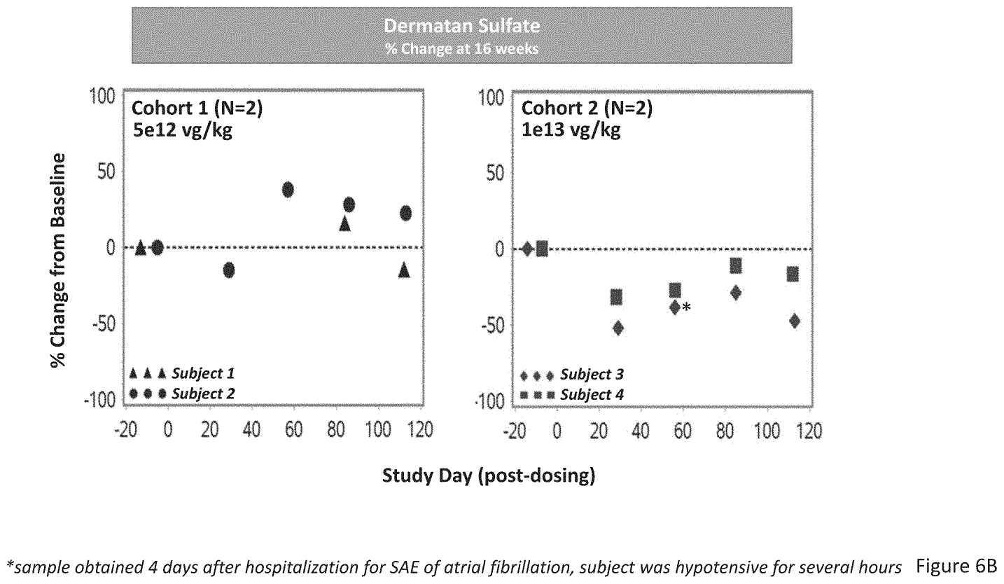

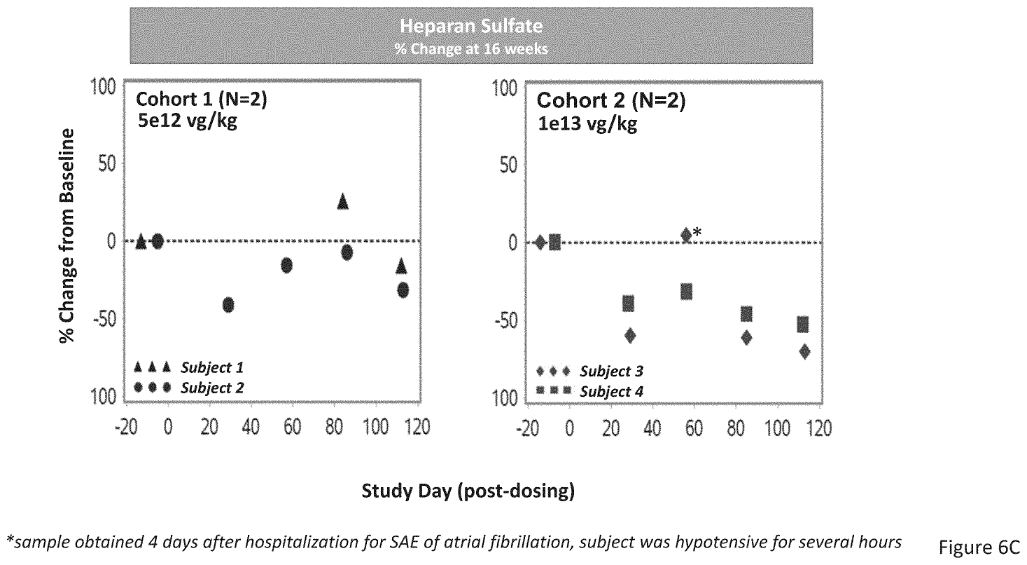

Furthermore, as shown in FIGS. 6A through 6C (which depict individual subject values at baseline and each monthly visit for each subject), the reduction in MPS II biomarkers post-treatment persisted over time. For both subjects in Cohort 2, total GAGs, dermatan sulfate and heparan sulfate observations remained below baseline throughout the sixteen weeks, with the exception of one time point when a sample was obtained four days after a subject was hospitalized for an SAE of atrial fibrillation, unrelated to study drug, and was hypotensive for several hours. At the next measurement, this patient's GAGs returned to the previous low range observed since week 4.

In contrast to Cohort 2, Cohort 1 subjects' GAGs generally remained around their baseline measurements. For one patient in cohort 1, total GAGs rose slightly, and for the other patient total GAGs declined slightly. The patients in this study are all on ERT, which means their GAGs are significantly reduced compared to ERT naive MPS II patients. The GAG levels of MPS II patients with attenuated disease who are on ERT can vary but usually within a range that diagnostic models would consider the lower end of diseased all the way to very low end of normal. The patients in Cohorts 1 and 2 had baseline GAG values in the high normal range, elevated enough to still have room for measurable declines. Plasma IDS activity was below the level of quantification at baseline and for the first 16 weeks post dosing with the composition. The absence of detectable levels does not indicate that IDS is not being produced by liver cells edited using the methods and compositions disclosed herein. It is possible that continuous exposure of cells to low levels of circulating IDS may be sufficient to drive enzyme uptake into cells and reduce or maintain suppression of GAGs as these tissues are starving for IDS. The sixth subject (cohort 3) has been dosed at 5.00e13 vg/kg and will be followed to observe the safety and exploratory endpoints described above. The patients are evaluated for withdrawal of ERT. Stabilization of urine GAG levels after ERT withdrawal will be one important parameter for evaluating therapeutic potential of the methods and compositions disclosed herein.

24-Week Urine GAG Results, Cohorts 1 Through 3

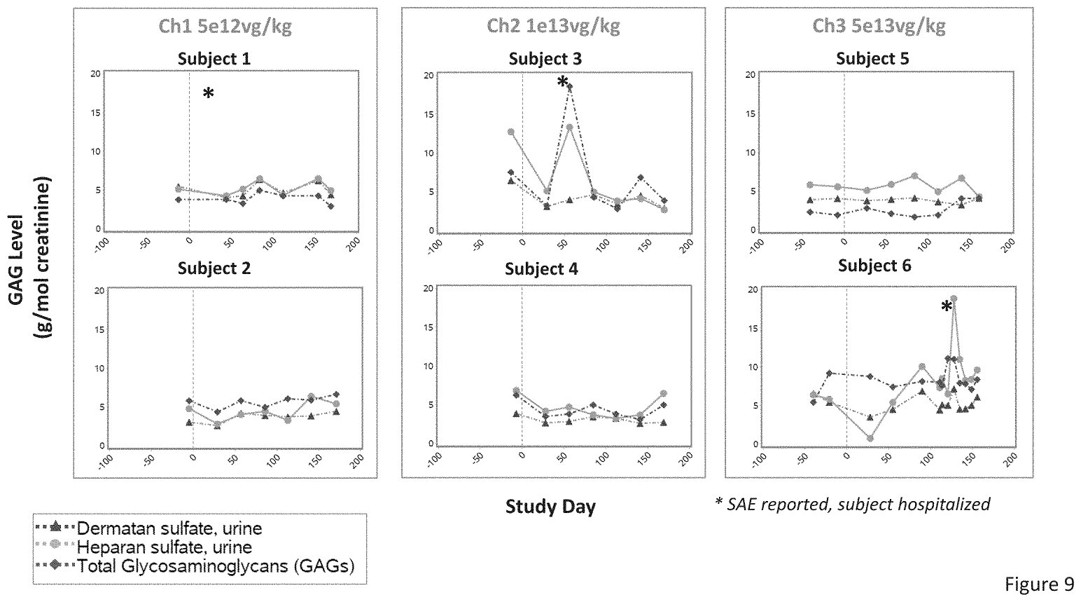

Urine GAGs, including dermatan sulfate, heparan sulfate and total GAGs were measured for three dose cohorts, including 5e12 vg/kg, 1e13 vg/kg and 5e13 vg/kg. 8 subjects have been treated but this 24 week data set includes only the first 6 subjects. The data is presented in FIG. 9.

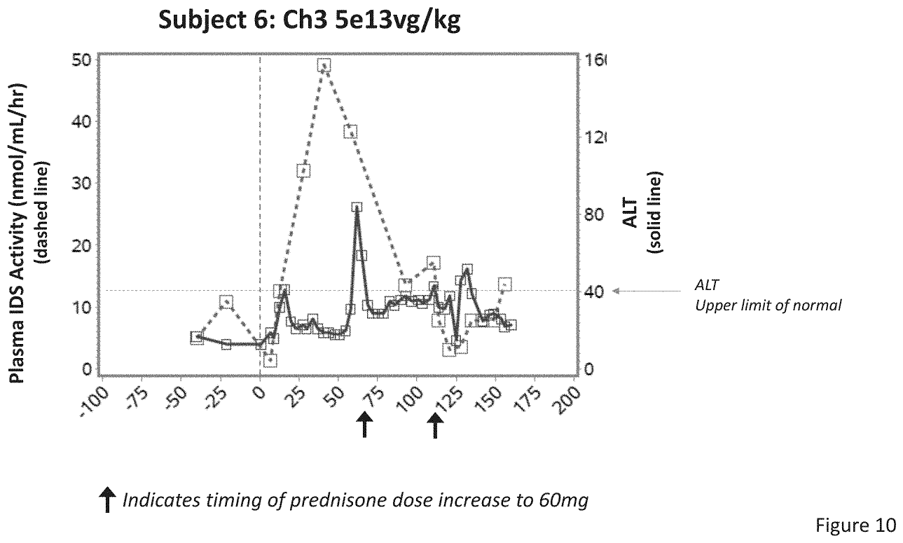

FIG. 8 demonstrates a large increase in plasma IDS in Subject 6 in cohort 3 at approximately day 50 post treatment and the development of transaminitis. FIG. 10 depicts a close up of Subject 6's IDS levels and the changes in liver alanine aminotransferase that were observed during the spike in IDS levels. Mild (Gradel) increases in liver function tests were reported on study days 62, 111 and 128. Prednisone doses were increased to 60 mg PO daily and then tapered. This subject also had a SAE (incarcerated umbilical hernia) on study day 121 that was unrelated to the study drug.

Summary of Results at 24 Weeks

Preliminary evidence of successful editing of the human genome was shown. Through results of PCR analysis—an assay to detect gene integration—of liver samples, two patients in Cohort 2, demonstrated gene integration. In addition, the fusion albumin IDS transcript was synthesized. These genome-edited liver cells generated IDS activity in the plasma of the treated MPS II patients. The patients summarized here were treated with the composition disclosed herein comprising AAV SB-47171, AAV SB47898, and AAV hIDS Donor.

ERT withdrawal has been initiated under the protocol-specified schedule while monitoring safety, IDS/GAG biochemical markers and functional measures. The response of urine GAG levels after ERT withdrawal is important to determine the potential clinical relevance of the study drug. Three subjects (2 in cohort 2 and 1 in cohort 3) have started ERT withdrawal, however, 1 subject in cohort 2 may restart ERT after approximately 3 months due to fatigue and concurrent increase in urine GAGs.

The study drug was administered to 8 subjects with attenuated MPS II at a dose of up to 5e13 vg/kg and was generally well tolerated. Adverse events related to study drug were mild or moderate and all resolved. Administration of the study drug was generally well-tolerated with a favorable safety profile in these patients. Seventeen adverse events were reported, 15 of which were mild, Grade 1, and all reported adverse events resolved without treatment. No serious adverse events related to study drug were reported. Liver biopsy results—from one patient in the low-dose cohort and two patients in the mid-dose cohort—indicate preliminary evidence of genome editing of liver cells in the two subjects treated at the 1e13 mid-dose. The analysis was done using an assay using a reverse transcriptase polymerase chain reaction (RT-PCR), a standard laboratory method used to amplify and detect specific molecular signals. The assay was designed to detect gene integration by identifying albumin-IDS chimeric mRNA transcripts, which can only be made if the IDS gene has successfully integrated at the expected site within the endogenous albumin gene. This assay detected albumin-IDS chimeric mRNA transcript in both mid-dose cohort patients. Genome editing in mid-dose cohort patients appears to be occurring at a low frequency, as a separate liver tissue analysis conducted using MiSeq DNA sequencing, a less sensitive assay (lower limit of quantification of 0.1%), did not detect editing in samples from the low and mid-dose cohort patients. Analysis of liver tissue showed evidence of albumin-IDS mRNA transcripts in both subjects at the 1e13 vg/kg dose after 24 weeks, indicating that genome editing occurred. Analysis of cohort 3 subjects is not complete. A substantial increase in plasma IDS activity was observed in one subject at the 5e13 vg/kg dose, however, this decreased after the development of mild transaminitis.

In addition, this summary also included plasma IDS activity measurements using a more sensitive quantitative IDS assay. The IDS enzyme data included plasma activity levels from the first six patients enrolled across all three cohorts of the study, at 24 weeks post-treatment compared to baseline. Enzyme assay analysis detected small increases in IDS activity in the plasma of the two subjects in the 1e13 mid-dose, and in one subject at the 5e13 high dose. Furthermore, a significant increase in plasma IDS activity was measured in the second patient treated at the 5e13 high dose, with plasma IDS levels rising to approximately 50 nmol/hour/mL by day 50 post-SB-913 treatment, which is approximately 60% of the lower limit of normal plasma IDS activity. Mild, Grade 1, elevations in liver function tests were measured in this patient at day 62, 111 and 128, consistent with a suspected transaminitis event, resulting in the dissipation of plasma IDS activity in this subject, down to 14 nmol/hour/mL at 24 weeks post-treatment.

Urine GAG measurements at 24 weeks were also collected and compared to baseline in these first 6 patients. All subjects enrolled had baseline urine GAG levels in a range considered at normal or slightly above normal, and at 24 weeks, urine GAG levels from the first six patients did not show a clear trend or meaningful change.

Additional studies were performed as described herein using the composition disclosed herein comprising AAV SB-71557 and AAV SB-71728 (in place of 47171 and 47898), along with an AAV hIDS Donor. In pre-clinical studies, AAV SB-71557 and AAV SB-71728 have demonstrated improved cutting efficiency (5- to 30-fold) and improved expression (5- to 20-fold) of IDS (see U.S. application Ser. No. 16/271,250), the enzyme deficient in the MPS II subjects treated as described herein.

### Example 4

Iduronate-2-Sulfatase Enzyme Assay

Exemplary laboratory procedures that may be utilized are conducted as follows. To detect IDS enzyme activity, there are many assays that can be used. An exemplary assay is as follows: Iduronate-2-sulfatase is a lysosomal enzyme that removes a sulfate residue from the 2′ position of an iduronic acid residue that is present in both heparan sulfate and dermatan sulfate. This assay used an artificial 4-MU substrate that contained a terminal iduronic acid. However, in order for the fluorescence of 4-MU to be released, the entire iduronic acid moiety must be removed from the substrate. The removal of iduronic acid was catalyzed by the α-iduronidase enzyme, and this can only occur after the removal of the sulfate residue by iduronate-2-sulfatase. Therefore, this assay was a two-step reaction. During the first step, endogenous iduronate-2-sulfatase was given the opportunity to cleave the 2′ sulfate residue from the iduronic acid residue at the end of the 4-MU substrate. During the second step, exogenous lysosomal enzymes (including α-iduronidase, but not iduronate-2-sulfatase) were added to the reaction. The α-iduronidase enzyme can remove the iduronic acid from any 4-MU substrate from which the 2′ sulfate residue has already been removed by endogenous iduronate-2-sulfatase. The removal of the terminal iduronic acid from the 4-MU substrate releases its fluorescence, which is observed using a fluorometer. However, if no endogenous iduronate-2-sulfatase enzyme is present within the patient sample, the 2′sulfate residue cannot be removed, which prevents the entire iduronic acid moiety from being removed, thereby quenching the fluorescence of the 4-MU substrate (Voznyi et al. (2001) J Inher Metab Dis 24:675-680).

Procedure: Plasma was separated via centrifugation from whole blood (heparinized, green-top or EDTA preserved purple-top). Plasma were separated from whole blood via centrifugation. After centrifugation the top, liquid layer (plasma) was carefully pulled or poured off and collected in a separate, appropriate collection tube. This tube containing plasma is frozen and sent packed in dry ice.

Frozen plasma samples were removed from freezer and thawed quickly at 37° C. water bath prior to dilution. Plasma samples were diluted 1:10 with substrate buffer (10 μL plasma+90 μL substrate buffer) in a separate microcentrifuge tube. In each patient/control tube, 10 μL diluted plasma+20 μL Hunter substrate were combined in a microplate and incubated in a 37° C. incubator for 3 hours. 50 μL Quenching solution (2× Mcilvaine buffer with 0.2% BSA and 1 μg/mL recombinant human α-L-Iduronidase) was added to each sample and the reaction plate was put back in the 37° C. incubator for 24 hours. 40 μL of each reaction was transferred to a flat white opaque plate and 100 μL stop buffer was added. Fluorescence signal was acquired using (365 nm excitation, 450 nm emission) plate reader. Total enzyme activity was determined using the following calculations:

Plasma: Average corrected reading x dilution factor (10)=nmoles of substrate hydrolyzed per 3 hours per mL plasma. Normal plasma values are from 82-200 nmol/hr/mL (determined from 50 donors). The lower limit of quantification (LLOQ) of enzyme activity is 0.78 nmol/mL/hr. The upper limit of the analytical measurement range for enzyme activity is 167 nmol/mL/hr.

Substrate buffer was prepared as follows: 0.1 M sodium acetate and was combined with 0.01M lead acetate and adjust to pH of 5.0 using glacial acetic acid. 0.2% BSA was added to substrate buffer on the day of use for sample dilution. Hunter substrate 4MU-αIdoA-2S (2.5 mM) was purchased commercial.

Quenching solution: 2× Mcilvaine buffer was prepared at 0.4M sodium-phosphate dibasic and 0.2M citrate, pH 4.5. 0.2% BSA was added to 2× Mcilvaine buffer on the day of use. Quenching solution was prepared by diluting recombinant human α-L-Iduronidase (R&D system) in 2× Mcilvaine buffer containing 0.2% BSA at final concentration of 1 μg/mL.

This assay has a lower limit of quantitation=0.78. Reference ranges (nmol/mL/hr) for unaffected individuals is 82-200, while baseline for MPS II patients (>96 h post-ERT) is estimated at 0-10.

Another exemplary assay to measure IDS activity in the blood is as follows: 4-Methylumbelliferyl α-L-idopyranosiduronic acid 2-sulfate (IDS-S) is used as a substrate for IDS. Its enzymatic product, 4-methylumbelliferyl α-L-idopyranosiduronic acid (IDS-P) and internal standard, 4-methylumbelliferyl α-L-idopyranoside (IDS-IS), are then directly measured by UPLC-MS/MS (Lee et al. (2015) Clin Biochem. 48(18):1350-3).

Total Urine Glycosaminoglycans (GAGs) Assay and Quantitative Urine Heparan Sulfate, Dermatan Sulfate and Chondroitin Sulfate Assay by MS/MS.

A variety of assays exist to measure the level of GAGs in the urine. One exemplary assay is described as follows: Urine samples are collected during the study are analyzed for glycosaminoglycan levels using a Dimethyl Methylene Blue (DMB) Assay. Briefly, urine samples are stained for heparan sulfate by treating the sample with 1,9-dimethylmethylene blue dye resuspended in formic acid at a pH of 3.3, and measured for absorbance at a wave length of 520 nm. The concentration of heparan sulfate was normalized using the total concentration of creatinine protein identified in the urine sample. (see e.g. de Jong et al. (1989) Clin Chem 35/7:1472-1479).

Another exemplary assay for measuring total GAG present in a biological sample is as follows: The method involves (a) combining a serine protease (e.g., of the clotting cascade), a labeled substrate for the serine protease, an inhibitor of the serine protease, and a sample suspected of comprising one or more glycosaminoglycans under conditions and for a time suitable for cleavage of the labeled substrate by the serine protease to produce a detectable signal, (b) detecting the detectable signal, and (c) comparing the amount of detectable signal with a standard to determine the concentration of said one or more glycosaminoglycans in said sample, wherein said inhibitor of said serine protease is selected from the group consisting of heparin cofactor II and antithrombin III, and wherein said one or more glycosaminoglycans are selected from the group consisting of dermatan sulfate (DS) and heparin sulfate (HS). (See e.g. U.S. Patent Publication No. 2013/0189718).

Another exemplary assay measures the types of GAGs present and is termed a multiplex assay (Langereis et al. (2015) PLoS One 10(9):e0138622). This assay is based on enzymatic digestion the of heparan sulfate (HS), dermatan sulfate (DS) and keratan sulfate (KS) found in the urine, followed by quantification by LC-MS/MS. This assay is a very sensitive assay and can be used to measure the exact types of GAGs in the urine.

Another exemplary assay that can be used to determine the concentration of specific types of GAGs utilizes a RapidFire (RF, Agilent) high-throughput mass spectrometry system. Samples are absorbed to a matrix to concentrate and desalt, and then eluted directly into the MS/MS without chromatographic separation. Each sample is processed in less than ten seconds, yielding much faster throughput than conventional LC-MS/MS based methods (see Tomatsu et al. (2014) J Anal Bioanal Tech. Mar. 1; 2014 (Suppl 2):006.)

AAV2/6 Clearance in Plasma, Saliva, Urine, Stool and Semen

Detection of AAV in biological samples can be done by several methods known in the art. An exemplary shedding assay is for analysis of AAV2/6-donor and AAV2/6-ZFN vectors in human plasma, semen, saliva, urine, and feces samples, and to evaluate the recovery rate of DNA from the five matrices. Human plasma, semen, saliva, urine, and feces samples from human donors provided the source of matrix DNA for qPCR analysis.

DNA isolation from human Plasma: An aliquot (200 μL) of human plasma sample was thawed, treated with proteinase K in the presence of 2 μg of salmon sperm DNA, prior to DNA isolation using QIAamp DNA Mini kit. The purified plasma DNA was dissolved in 100 μL of elution buffer AE.

DNA isolation from human semen: An aliquot (up to 100 μL) of human semen sample was thawed, treated with proteinase K, and then processed for DNA isolation using QIAamp DNA Mini kit. The purified semen DNA was dissolved in 100 μL of elution buffer AE and the DNA concentration was determined by UV absorption at 260 nm with Nanodrop ND-8000 instrument.

DNA isolation from human saliva: An aliquot (up to 200 μL) of human saliva sample was thawed, treated with proteinase K, and then processed for DNA isolation using QIAamp DNA Mini kit. The purified saliva DNA was dissolved in 100 μL of elution buffer AE and the DNA concentration was determined by UV absorption at 260 nm with Nanodrop ND-8000 instrument.

DNA isolation from human urine: An aliquot (up to 200 μL) of human saliva sample was thawed, treated with proteinase K, and then processed for DNA isolation using QIAamp DNA Mini kit. The purified saliva DNA was dissolved in 100 μL of elution buffer AE and the DNA concentration was determined by UV absorption at 260 nm with Nanodrop ND-8000 instrument.

DNA isolation from human feces: An aliquot (90-110 mg) of human feces sample was partially thawed, homogenized, and treated with proteinase K prior to DNA isolation using QIAamp Fast DNA Stool Mini Kit. The purified feces DNA was dissolved in 200 μL of Buffer ATE and the DNA concentration was determined by UV absorption at 260 nm with Nanodrop ND-8000 instrument.

Each qPCR was performed on a standard 96-well plate in a 7900HT Fast Real Time PCR system. The plate with reaction mix was sealed with optical caps and all droplets spun down by centrifugation at 1500 rpm for 15 min before qPCR.

The reaction for the donor AAV (SB-IDS, SB-A6P-HNT) amplified and detected a 91 nucleotide amplicon. The reaction for detection of the ZFN DNA (SB-47171: SB-A6P-ZLEFT and SB-47898: SB-A6P-ZRIGHT) amplified and detected a 96 nucleotide amplicon.

Assay conditions used: Held at 50° C. for 2 minutes. Held at 95° C. for 10 minutes. 40 cycles at 95° C. for 15 seconds, and at 60° C. for 1 minute. Results were compared with a previously prepared standard curve using linearized MPS II or ZFN plasmid DNA.

Gene Modification at the Albumin Locus in Liver Tissue

An exemplary assay for detection of gene modification through sequencing is to determine the levels of insertions and deletions (indels) at the albumin gene in subject samples using the MiSeq next generation sequencing (NGS) platform. gDNA was isolated from liver tissue using standard procedures and diluted to 20 ng/mL. Samples were subjected to an adaptor PCR followed by a barcode PCR and loaded onto MiSeq cartridge for sequencing. Following conditions are used for PCR reactions:

PCR reaction (Adaptor): 95° C. 3 minutes, [98° C. 20 seconds, 55° C. 15 seconds, 72° C. 15 seconds], repeat bracketed steps 29 times. Final extension at 72° C. for 1 minute.

PCR reaction (Barcode): 95° C. 3 minutes, [98° C. 20 seconds, 60° C. 15 seconds, 72° C. 15 seconds], repeat bracketed steps 9 times. Final extension at 72° C. for 1 minute.

Identification of Albumin-IDS mRNA Transcript in Liver Biopsy

An RT-PCR method was developed to assay the presence of a unique albumin-IDS mRNA transcript that occurs as a result of insertion of the IDS gene into the albumin locus. This assay was used to test liver biopsy tissue from subjects who had been treated with study drug. In brief, RNA was extracted from frozen liver tissue using a FastPrep® Instrument (MP Biomedicals) using the manufacture's RNA liver settings. RNA is isolated using a PureLink RNA mini kit (Thermo Fischer) according to manufacturer's protocol. RNA was quantitated and used for reverse transcription using a SuperScript III First-strand synthesis system (Invitrogen) according to manufacturer's protocols. Following reverse transcription, the samples were subjected to PCR using the following sets of primers:

Probes and Primers for Albumin-IDS mRNA Assay

| | | SEQ |
|---|---|---|
| | | ID |
| Name | Sequence (5′ -> 3′) | NO |
| IDS-ALB_F2 | CTCTTTAGCTCGGCTTATTCCA | 40 |
| IDS-ALB_R2 | CGATGATGAGCAGGACGTTAAG | 41 |
| ALB_IDS_Probe2: | TCGAGATGCACTTAGCGAAACCCA | 42 |
| 6-FAM/ZEN/IBFQ | | |
| ALB_F2 | TTCGTCGAGATGCACACAAG | 43 |
| ALB_R2 | TGCTGAAGATACTGAGCAAAGG | 44 |
| ALB_Probe2 | AGGTTGCTCATCGGTTTAAAGATTTGGGA | 45 |
| 6-FAM/ZEN/IBFQ | | |

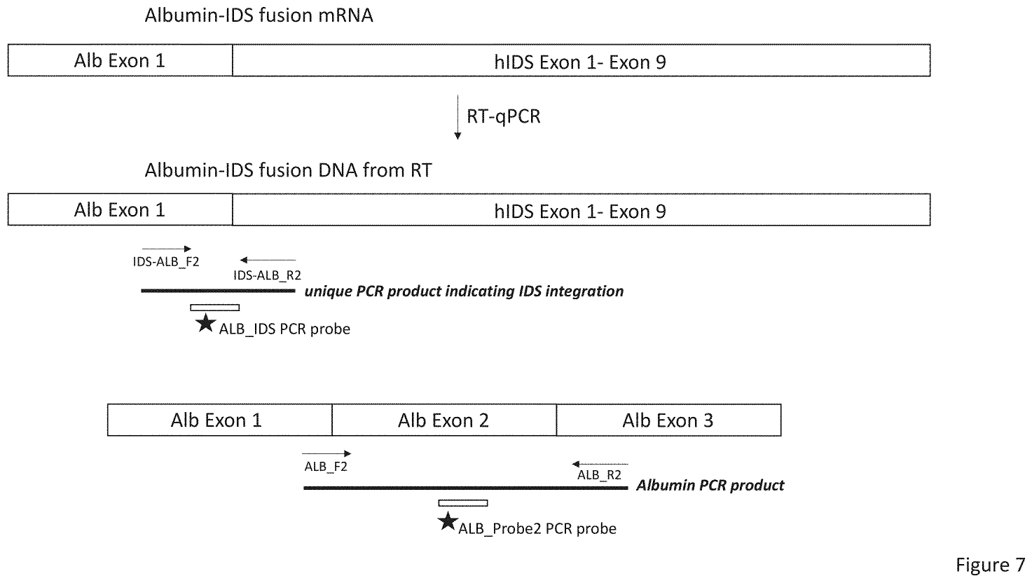

The primers and probes were used as depicted in FIG. 7 to amplify either a unique PCR product following IDS integration into the albumin gene, or to detect albumin alone, without IDS integration.

Quantitation of the PCR products was done using a LabChipGX Touch HT system (Perkin Elmer). The lower limit of detection for this assay is approximately 1 in 1,000 genomes.

### Example 5

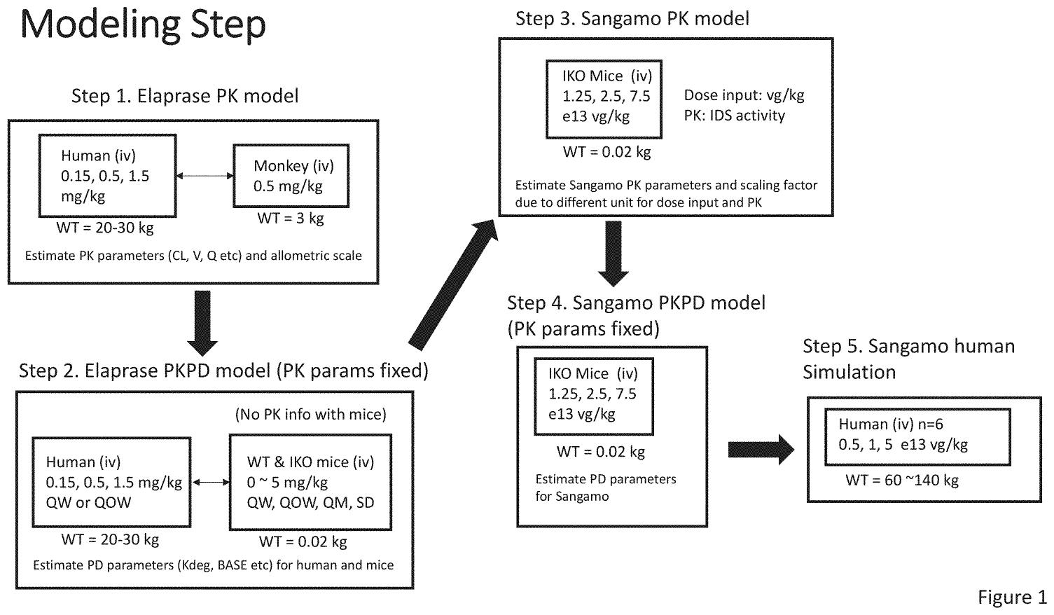

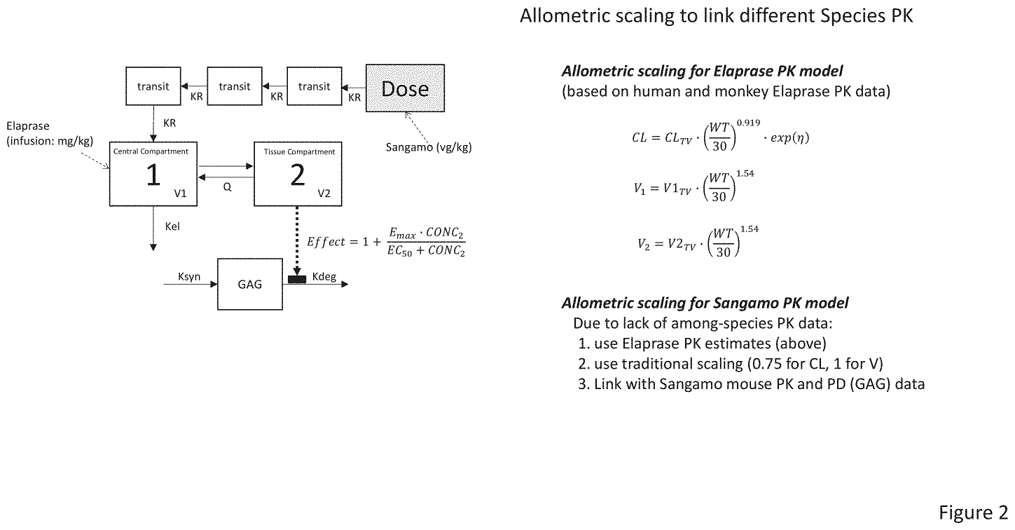

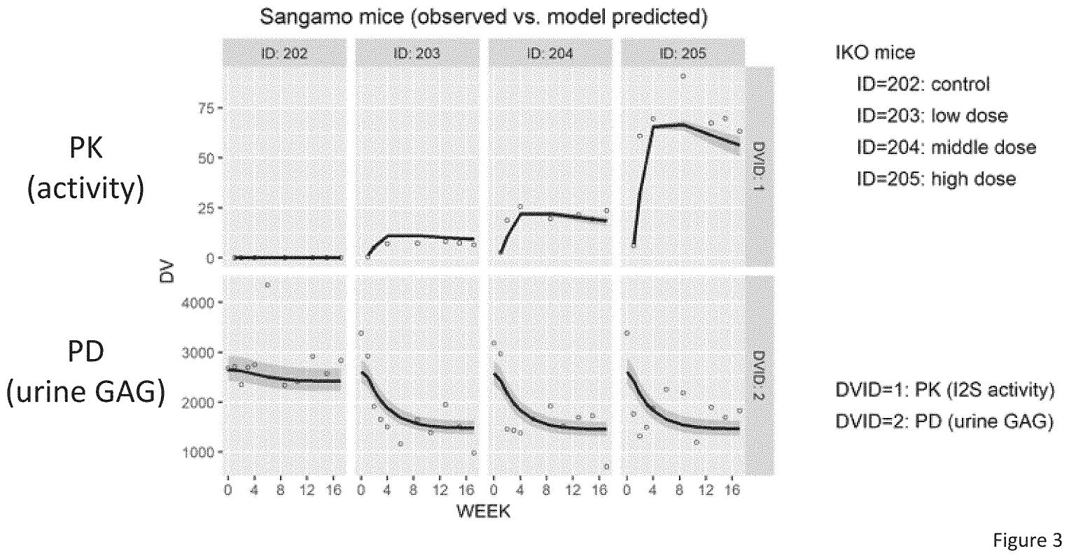

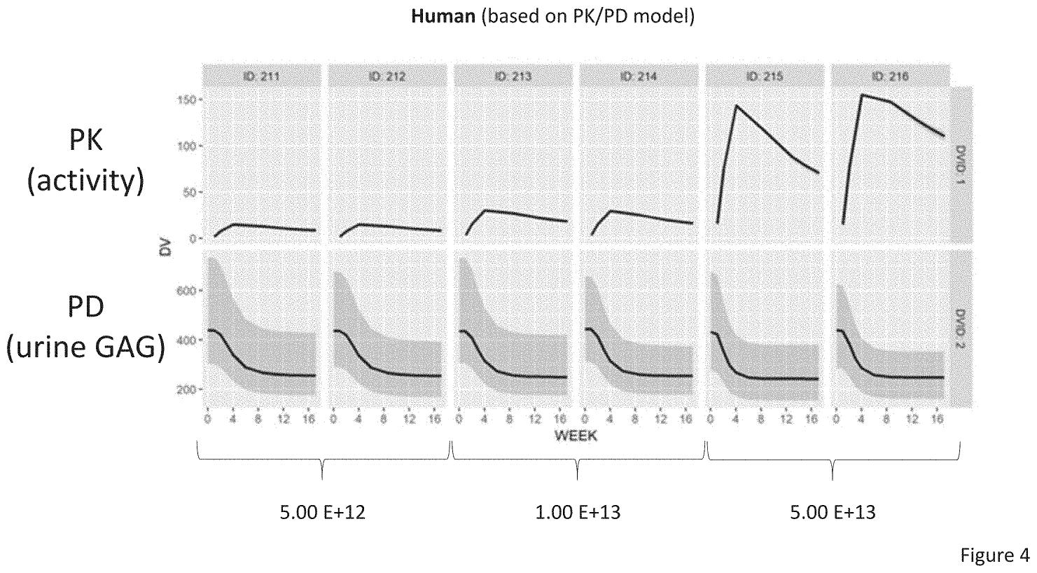

Models to determine a minimally efficacious dose can be developed to predict an efficacious dose in a subject treated with a composition of the invention. Models were developed to examine the predicted pharmacokinetics (PK) and pharmacodynamics (PD) of a composition to predict the therapeutically efficacious dose. Published PK data (Tomanin et al. (2014) Orphanet J Rare Dis. 9:129.; Garcia et al. (2007) Mol Genet Metab. 91(2):183-90; Kim, S et al. (2017) Mol Genet Metab.122(1-2):92-99. doi: 10.1016/j.ymgme.2017.06.001.; Sohn et al. (2013) Orphanet J Rare Dis. 8:42.; Kim, C et al. (2017) J Hum Genet. 62(2):167-174.; Elaprase FDA Pharmacology/Toxicology and Medical Review Documents) using Elaprase® in human and non-human primates, and PD and PK data from mice studies on the compositions of the invention (Ou, L. et al., manuscript in preparation) was fit. First, a PK/PD model for Elaprase® using human, monkey and IDS knock-out mice (reference) was developed. Next, a PK/PD model was developed using the compositions of the invention in IDS knock-out mice (Ou et al. (2014) Mol Genet Metab. 111(2):116-122). Then, the two models were linked to simulated the behavior of the composition of the invention in humans (FIG. 1), where a set of calculations were performed to mathematically link the behavior of the two therapeutics (FIG. 2). The model was applied to the mouse observed data to align to the predicted values (FIG. 3). Next, the model was used to predict the efficacious dose required for plasma IDS activity that would result in lowering urine GAG levels. As shown (FIG. 4), even the lowest dose of 5.0 e+12 vg/kg is predicted to lower urine GAGs in a treated subject because it is predicted to result in an IDS plasma activity above the lowest estimated efficacious dose (see below).

Literature searches found that the amount of IDS activity (as measured by the GCC assay) in normal humans, MPS-II carriers (heterozygotes) and affected subjects is as shown in the table below:

TABLE 9B
Reference values for IDS activity

| MPS II-IDS | Fibroblasts | Leukocytes | Plasma |
|---|---|---|---|
| Normal | 26-208 nmol/4 hr/mg (1) | 18-57 nmol/4 hr/mg (1) | 167-475 nmol/4 hr/mL(1) |
| | | 31-110 nmol/4 h/mg (4) | 155-1082 nmol/4 hr/mL (2) |
| | | 43 +/− 13.9 nmol/hr/mg (5) | 122-463 nmol/4 hr/mL (4) |
| | | 30.5-94 nmol/4 hr/mg (6) | 147-250 nmol/4 hr/mL (6) |
| | | | 316 +/− 126 nmol/4 hr/mL (8) |
| Carrier | | 9-54 nmol/4 hr/mg (4) | 44-202 nmol/4 hr/mL (4) |
| | | 17.5 +/− 5.7 nmol/hr/mg (5) | |
| | | 18-35 nmol/4 hr/mg (6) | 98-159 nmol/4 hr/mL (6) |
| Affected | 0-3.5 nmol/4 hr/mg (1) | 0-0.7 nmol/4 hr/mg (1) | 0-1.1 nmol/4 hr/mL (1) |
| | | 0.9 +/− 0.6 nmol/hr/mg (5) | 0-15 nmol/4 hr/mL (2) |
| | | 0-4.3 nmol/4 h/mg—very attenuated (7) | 0-7.46 nmol/4 hr/mL - severe (3) |
| | | | 0.31-8.18 nmol/4 hr/mL - attenuated (3) |
| | | | 4.15-22.1 nmol/4 hr/mL - attenuated (8) |

The source of the data shown in the above table is as follows: “(1)” is Voznyi et al. (2001) J. Inherit Metab Dis 24(6):675-80; “(2)” Greenwood Genetic Center, internal observations; “(3)” is Lee et al. (2015) Clin Biochem 48(18):1350-3; “(4)” is de Camargo Pinto et al. (2010) Am J Med Genet A.; “(5)” is Lin et al. (2006) Clin Chim Acta 369(1): 29-34; “(6)” is Schwartz et al. (2009) J Inherit Metab Dis 32(6):732-738; “(7)” is Quaio et al. (2012) J Inherit Metab Dis 4:125-8; and “(8)” is Chistiakov et al. (2014) J. Genet Genomics 41(4): 197-203.

It has been estimated that 30% of systemic IDS will be internalized by cells, and 25% of normal IDS is sufficient for correction of the MPS-II phenotype (Eliahu et al. (1981) Am J Hum Genet 33(4):576-83). The activity observed in leukocytes isolated from “carriers” the MPS-II phenotype (see Table 9B) is 9-54 nmol/4 hr/mL. Thus, if 30% of IDS in the plasma is internalized into the cells, a plasma concentration of 20-30 nmol/4 hr/mL, resulting in an internal cellular concentration of approximately 6.5 nmol/4 hr/mg-10 nmol/4 hr/mg is sufficient to alleviate the symptoms associated with the disease.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method of reducing, delaying and/or eliminating: the need for additional treatment procedures, the onset, progression and/or severity of symptoms in a subject with MPS II, the method comprising treating the subject by administering a composition comprising first, second and third AAV vectors, the first AAV vector comprising a sequence encoding a left ZFN designated 71557 or 47171, the second AAV vector comprising a sequence encoding a right ZFN designated 71728 or 47898 and the third AAV vector comprising a sequence encoding iduronate 2-sulfatase (IDS).

2. The method of claim 1, wherein GAG levels in the subject are reduced, stabilized and/or GAGs are eliminated from the urine of the subject.

3. The method of claim 1, wherein IDS levels in the plasma and/or leukocytes are stabilized and/or increased, optionally wherein IDS levels stay the same or is below the level of detection.

4. The method of claim 1, wherein first, second and third AAV vectors are administered at a fixed ratio of 1:1:8.

5. The method of claim 1, wherein the additional treatment procedures that are reduced, delayed, and/or eliminated comprise enzyme replacement therapy (ERT); bone marrow transplant; and/or one or more supportive surgical procedures for orthopedic, cardiac and/or upper airway obstruction, wherein cardiac and/or upper air obstruction includes adenotonsillectomy, hernia repair, ventriculoperitoneal shunt, cardiac valve replacement, carpal tunnel release, and/orspinal decompression.

6. The method of claim 1, wherein the symptoms associated with MPS II whose onset, progression or severity are reduced, delayed or eliminated comprise a decline in functional abilities, neurologic deterioration, joint stiffness, becoming wheelchair dependent, progression of disability, the requirement for forced air positive ventilation and/or a shortened life span.

7. The method of claim 1, wherein the first and/or second AAV vectors comprise(s) one or more of the following sequences: sequences encoding small peptides (including but not limited to peptide tags such as FLAG or His tag sequences); a WPRE sequence; a nuclear localization signal (NLS)-encoding sequence; a polyA signal; one or more mutations in one or more of the zinc finger protein of the zinc finger nuclease; one or more mutations in a FokI nuclease cleavage domain or cleavage half domain of the zinc finger nuclease; a promoter sequence that drives expression of the ZFN; one or more intron sequences; and/or one or more enhancer sequences.

8. The method of claim 1, wherein:
the left ZFN comprises 71557 and the right ZFN comprises 71728; or
the left ZFN comprises SB-A6P-ZL2 and the right ZFN comprises SB-A6P-ZR2; or
the left ZFN comprises 47171 and the right ZFN comprises 47898; or
The left ZFN comprises SB-A6P-ZLEFT and the right ZFN comprises SB-A6P-ZRIGHT.

9. The method claim 1, wherein the IDS donor comprises a human IDS-encoding sequence.

10. The method of claim 9, wherein the IDS donor comprises the sequence as shown in SEQ ID NO:15 and/or an AAV vector comprising: (i) the sequences as shown in Table 3 or (ii) the sequence as shown in SEQ ID NO:17.

11. The method of claim 1, further comprising measuring IDS activity and/or level in the plasma, liver, CSF or in leukocytes in the subject before and after treatment, wherein additional therapeutic procedures are delayed, reduced or eliminated if IDS activity is increased after treatment.

12. The method of claim 1, further comprising measuring total GAG levels, GAG comprising dermatan sulfate (DS GAG) levels, and/or GAG comprising heparan sulfate (HS GAG) levels (in the urine of the subject before and after treatment, wherein additional therapeutic procedures are delayed, reduced or eliminated if GAG, DS GAG and/or HS GAG levels are reduced after treatment.

13. The method of claim 1, further comprising measuring forced vital capacity before and after treatment, wherein additional therapeutic procedures are delayed, reduced or eliminated if pulmonary function is increased after treatment.

14. The method of claim 1, further comprising measuring distance walked before and after treatment, wherein additional therapeutic procedures are delayed, reduced or eliminated if distance walked is increased after treatment.

15. The method of claim 1, further comprising measuring joint range of motion (JROM) before and after treatment, wherein additional therapeutic procedures are delayed, reduced or eliminated if JROM is increased after treatment.

16. The method of claim 1, further comprising measuring spleen and/or liver volume before and after spleen and/or liver volume is increased after treatment.

17. The method of claim 1, further comprising measuring one or more neurocognitive abilities before and after treatment, wherein additional therapeutic procedures are delayed, reduced or eliminated if one or more of the neurocognitive abilities is increased after treatment.

18. The method of claim 1, wherein disability progression, organomegaly, hyperactivity, aggressiveness, neurologic deterioration, joint stiffness, skeletal deformities, heart valve thickening, hearing loss, corneal clouding and vision impairment, hernias, and/or upper respiratory infections are suppressed, reduced, delayed or eliminated in the subject after treatment.

19. The method of claim 1, wherein the need for the use of a medical ventilator device in the subject is stabilized, delayed, reduced or prevented after treatment.

20. The method of claim 1, wherein the onset of the subject being wheelchair dependent is delayed, reduced or prevented after treatment.

21. The method of claim 1, wherein the life expectancy of the subject is increased after treatment.

22. The method of claim 1, wherein the additional therapeutic procedure is ERT, wherein ERT is reduced or withdrawn after treatment.

23. The method of claim 1, wherein the additional therapeutic procedure is a bone marrow transplant.

24. The method of claim 1, wherein the subject receives a total AAV dose, of between 1e12 and 1e16 vg/kg.

25. The method of claim 24, wherein the total AAV dose comprises:
(i) 5e12 vg/kg comprising 5e11 vg/kg of the first and second AAV vectors and 4e12 vg/kg of the third AAV vector;
(ii) 1e13 vg/kg comprising 1e12 vg/kg of the first and second AAV vectors and 8e12 vg/kg of third AAV vector;
(iii) 5e13 vg/kg comprising 5e12 vg/kg of the first and second AAV vectors and 4e13 of third AAV vector;
(iv) 1e14 vg/kg comprising 1e13 vg/kg of the first and second AAV vectors and 8e13 vg/kg of the third AAV vector;
(v) 5e14 vg/kg comprising 5e13 vg/kg of the first and second AAV vector and 4e14 vg/kg of the third AAV vector; or
(vi) 1e15 vg/kg comprising 1e14 vg/kg of the first and second AAV vectors and 8e14 vg/kg of the third AAV vector.

26. The method of claim 1, wherein the composition is administered intravenously, optionally via an infusion pump at a rate of anywhere between 10 to 200 mL/hour.

27. The method of claim 26, wherein the rate of infusion is 100 mL/hour.

28. The method of claim 1, wherein the subject is premedicated with a corticosteroid, prior to and/or after treatment with the composition.

29. The method of claim 28, wherein the corticosteroid is prednisone.

30. The method of claim 29, wherein the subject is treated one or more prior to treatment; the day of treatment; on day 7 after treatment, weekly after treatment and/or every other week up to 20 weeks after treatment.

31. The method of claim 1, wherein the subject is an adult or child with Hunter syndrome, wherein Hunter syndrome includes early onset MPS II, attenuated MPS II or MPS II between early onset and attenuated.

32. The method of claim 1, wherein the composition comprises an article of manufacture comprising a formulation that includes three pharmaceutical compositions comprising the first, second and third AAV vectors.

33. The method of claim 32, wherein each pharmaceutical composition is labeled with a different color.

34. The method of claim 32, wherein the pharmaceutical compositions are combined prior to administration to the subject.

35. The method of claim 1, wherein the total dose for the subject is determined as follows: determining the subject's weight rounded to two decimal pointsbefore treatment; dividing the subject's weight by the vg/mL concentration, thereby determining the dose to be used.

36. The method of claim 35, wherein the method comprises
(i) calculating the three product component volumes by multiplying the cohort dose by the patient weight before treatment and then dividing by the VG concentration as follows:
(a) obtaining the cohort and patient weight at baseline from the study coordinator
(b) obtaining the VG concentrations from the Clinical Certificates of Analysis;
(ii) calculating the total volume by adding together the three product component volumes and the NS/PBS volume;
(iii) calculating the volume of HSA intravenous solution required to achieve a final concentration of 0.25% HSA, and
(iv) calculating the adjusted NS/PBS volume.


SEQUENCE LISTING (48 sequences)

SEQ ID NO: 1 - DNA, 130 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 723 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 3 - DNA, 133 bp, Artificial Sequence
  Description of Artificial Sequence: Synthetic polynucleotide
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 4 - DNA, 21 bp, Artificial Sequence
  Description of Artificial Sequence: Synthetic oligonucleotide
cccaagaagaagaggaaggtg

SEQ ID NO: 5 - DNA, 432 bp, Artificial Sequence
  Description of Artificial Sequence: Synthetic polynucleotide
gccgcaatggcagaacggcccttccagtgccgcatctgcatgcgcaacttcagccagtcgggcaacctgtcccgccacatccggactcataccggcgaaaaaccattcgcttgtgacatctgcggaagaaagtttgcgctgaagcagaacctctgcatgcataccaagattcacaccggagagaagccgtttcagtgtcgcatttgcatgagaaagttcgcctgggccgataaccttcagaatcacaccaagatccacaccggggaaaagccgttccagtgccggatctgcatgaggaacttctcaacgtccggaaacctgaccaggcatatccggacccacactggggagaagcctttcgcctgcgacatttgcggtcggaagttcgcccggcaatcccacttgtgtctccacactaagatccacctgaga

SEQ ID NO: 6 - DNA, 600 bp, Artificial Sequence
  Description of Artificial Sequence: Synthetic polynucleotide
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttcagatcttgataa

SEQ ID NO: 7 - DNA, 223 bp, Artificial Sequence
  Description of Artificial Sequence: Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctat

SEQ ID NO: 8 - DNA, 108 bp, Artificial Sequence
  Description of Artificial Sequence: Synthetic polynucleotide
aggaacccctagtgatggagttggccactccctctctgcgcgctcgctcgctcactgaggccgcccgggctttgcccgggcggcctcagtgagcgagcgagcgcgcag

SEQ ID NO: 9 - DNA, 2529 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggcaattcgccatggcccccaagaagaagaggaaggtgggcatccacggggtaccggccgcaatggcagaacggcccttccagtgccgcatctgcatgcgcaacttcagccagtcgggcaacctgtcccgccacatccggactcataccggcgaaaaaccattcgcttgtgacatctgcggaagaaagtttgcgctgaagcagaacctctgcatgcataccaagattcacaccggagagaagccgtttcagtgtcgcatttgcatgagaaagttcgcctgggccgataaccttcagaatcacaccaagatccacaccggggaaaagccgttccagtgccggatctgcatgaggaacttctcaacgtccggaaacctgaccaggcatatccggacccacactggggagaagcctttcgcctgcgacatttgcggtcggaagttcgcccggcaatcccacttgtgtctccacactaagatccacctgagaggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttcagatcttgataactcgagtctagaggatctcgagccgaattcctgcagcccgggggatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagggccgcaggaacccctagtgatggagttggccactccctctctgcgcgctcgctcgctcactgaggccgcccgggctttgcccgggcggcctcagtgagcgagcgagcgcgcag

SEQ ID NO: 10 - DNA, 516 bp, Artificial Sequence
  Description of Artificial Sequence: Synthetic polynucleotide
gccgcaatggcagagaggccctttcagtgccggatctgcatgcggaacttctccaccccacaacttctggaccgacatatccgcacccataccggggaaaagcctttcgcgtgcgacatttgcggacggaaattcgcgttgaagcacaatctcctgacccacactaagattcatactggcgaaaagccgttccagtgccgcatctgtatgaggaacttcagcgatcagtcgaacctgaacgcccacattcggactcataccggagaaaagccctttgcctgcgatatctgcggtcgcaagttcgctaggaacttctcactgaccatgcacaccaaaatccacactggagagcggggattccagtgtagaatctgtatgcgcaacttctccctgcggcacgacctggaccgccacatcagaacccacaccggggagaagccgttcgcctgcgacatctgcggccggaagttcgcccaccggtccaacctgaacaagcacacgaagattcacctccgc

SEQ ID NO: 11 - DNA, 594 bp, Artificial Sequence
  Description of Artificial Sequence: Synthetic polynucleotide
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataa

SEQ ID NO: 12 - DNA, 2607 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggcaattcgccatggcccccaagaagaagaggaaggtgggcatccacggggtaccggccgcaatggcagagaggccctttcagtgccggatctgcatgcggaacttctccaccccacaacttctggaccgacatatccgcacccataccggggaaaagcctttcgcgtgcgacatttgcggacggaaattcgcgttgaagcacaatctcctgacccacactaagattcatactggcgaaaagccgttccagtgccgcatctgtatgaggaacttcagcgatcagtcgaacctgaacgcccacattcggactcataccggagaaaagccctttgcctgcgatatctgcggtcgcaagttcgctaggaacttctcactgaccatgcacaccaaaatccacactggagagcggggattccagtgtagaatctgtatgcgcaacttctccctgcggcacgacctggaccgccacatcagaacccacaccggggagaagccgttcgcctgcgacatctgcggccggaagttcgcccaccggtccaacctgaacaagcacacgaagattcacctccgcggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaggatctcgagccgaattcctgcagcccgggggatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagggccgcaggaacccctagtgatggagttggccactccctctctgcgcgctcgctcgctcactgaggccgcccgggctttgcccgggcggcctcagtgagcgagcgagcgcgcag

SEQ ID NO: 13 - DNA, 280 bp, Artificial Sequence
  Description of Artificial Sequence: Synthetic polynucleotide
tttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaa

SEQ ID NO: 14 - DNA, 28 bp, Artificial Sequence
  Description of Artificial Sequence: Synthetic oligonucleotide
actaaagaattattcttttacatttcag

SEQ ID NO: 15 - DNA, 1575 bp, Artificial Sequence
  Description of Artificial Sequence: Synthetic polynucleotide
agcgaaacccaggccaactcaactacagatgcgcttaacgtcctgctcatcatcgtggacgatttgcggccgtcgcttggctgctatggagataagctcgtccgctcgccgaacatcgatcagttggcctcacactcactgcttttccaaaatgcgtttgcgcagcaggctgtctgtgcaccttcaagagtctcattcttgaccgggcgacgccctgacacaacgcggctgtacgacttcaacagctactggagagtccacgcgggtaacttttcaactatcccacagtactttaaagagaacggatacgtgacaatgagcgtgggaaaggtctttcaccccggcatctcctcgaatcacaccgacgattcgccctactcgtggtcgtttcctccctaccatccttcgagcgagaagtatgagaacacgaaaacttgtcgcggacccgacggagagctgcacgctaatctgctgtgtccggtggatgtcttggacgtgcccgagggaacgctccccgacaagcagtcaacggagcaggcgattcagttgctggagaagatgaaaacaagcgcgtcgcctttcttcctcgccgtggggtatcacaagccccatattcctttccgctacccgaaggagttccagaaactttatcctttggaaaacatcactttggcaccggacccggaagtccccgacggtctgccacccgtggcctacaatccctggatggatatcaggcagagggaagatgtgcaggcactcaacatctcagtcccctacgggcctattccagtcgattttcaacgcaagattcggcagtcgtattttgcgtcggtgtcctacctcgatacgcaagtaggtcgacttctgagcgcgcttgatgaccttcagctggcaaattccacaatcatcgcctttacgtcggaccatgggtgggcgttgggagagcatggagagtgggcaaagtatagcaattttgatgtagcaacgcacgtgcccctgattttctacgtgccgggtagaacggcctcgcttcccgaggcaggcgaaaaactttttccctatctcgatccattcgactcggcgagccagcttatggaaccgggcagacaatccatggacttggtagaattggtgtccctttttccgaccctcgccgggttggcgggcttgcaagtaccccctagatgccctgtaccgagcttccatgtggaactctgccgcgaagggaaaaacctcctcaaacactttcggttcagggaccttgaggaggacccctatctgccagggaatccgcgagagttgattgcctattcccagtatccgcgacccagcgatattcctcaatggaactccgataagccctccctcaaagacatcaagattatggggtactcgatcaggaccatcgactatcgctacacagtgtgggtagggttcaatcctgacgaattcctcgcgaacttttcggacatccacgctggtgagctgtatttcgtagactcggacccgttgcaagatcacaatatgtataatgattcccaaggaggagatttgttccagctgctcatgccg

SEQ ID NO: 16 - DNA, 100 bp, Artificial Sequence
  Description of Artificial Sequence: Synthetic polynucleotide
ctatccattgcactatgctttatttaaaaaccacaaaacctgtgctgttgatctcataaatagaacttgtatttatatttattttcattttagtctgtct

SEQ ID NO: 17 - DNA, 2758 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagcggagttccaattgtactgtacagaaccatggtcacatgtttaacgctagcgtgccgacctggtaaactgatcagtgggtgcacttaggactgcgtcttacgctaatcacatgcgtgcggccgctttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaggtaccactaaagaattattcttttacatttcagttagcgaaacccaggccaactcaactacagatgcgcttaacgtcctgctcatcatcgtggacgatttgcggccgtcgcttggctgctatggagataagctcgtccgctcgccgaacatcgatcagttggcctcacactcactgcttttccaaaatgcgtttgcgcagcaggctgtctgtgcaccttcaagagtctcattcttgaccgggcgacgccctgacacaacgcggctgtacgacttcaacagctactggagagtccacgcgggtaacttttcaactatcccacagtactttaaagagaacggatacgtgacaatgagcgtgggaaaggtctttcaccccggcatctcctcgaatcacaccgacgattcgccctactcgtggtcgtttcctccctaccatccttcgagcgagaagtatgagaacacgaaaacttgtcgcggacccgacggagagctgcacgctaatctgctgtgtccggtggatgtcttggacgtgcccgagggaacgctccccgacaagcagtcaacggagcaggcgattcagttgctggagaagatgaaaacaagcgcgtcgcctttcttcctcgccgtggggtatcacaagccccatattcctttccgctacccgaaggagttccagaaactttatcctttggaaaacatcactttggcaccggacccggaagtccccgacggtctgccacccgtggcctacaatccctggatggatatcaggcagagggaagatgtgcaggcactcaacatctcagtcccctacgggcctattccagtcgattttcaacgcaagattcggcagtcgtattttgcgtcggtgtcctacctcgatacgcaagtaggtcgacttctgagcgcgcttgatgaccttcagctggcaaattccacaatcatcgcctttacgtcggaccatgggtgggcgttgggagagcatggagagtgggcaaagtatagcaattttgatgtagcaacgcacgtgcccctgattttctacgtgccgggtagaacggcctcgcttcccgaggcaggcgaaaaactttttccctatctcgatccattcgactcggcgagccagcttatggaaccgggcagacaatccatggacttggtagaattggtgtccctttttccgaccctcgccgggttggcgggcttgcaagtaccccctagatgccctgtaccgagcttccatgtggaactctgccgcgaagggaaaaacctcctcaaacactttcggttcagggaccttgaggaggacccctatctgccagggaatccgcgagagttgattgcctattcccagtatccgcgacccagcgatattcctcaatggaactccgataagccctccctcaaagacatcaagattatggggtactcgatcaggaccatcgactatcgctacacagtgtgggtagggttcaatcctgacgaattcctcgcgaacttttcggacatccacgctggtgagctgtatttcgtagactcggacccgttgcaagatcacaatatgtataatgattcccaaggaggagatttgttccagctgctcatgccgtgataaagatctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtctatccattgcactatgctttatttaaaaaccacaaaacctgtgctgttgatctcataaatagaacttgtatttatatttattttcattttagtctgtctggatccacaaattaatcgaacctgcagctgatatcgacgcttaagtagggcttagcaaacgcgtctccaacgtttcgccgttaacaccccacatagtgagtggtcttagtagtccgggtgtttaaactgaaagataactcgagcgcaggaacccctagtgatggagttggccactccctctctgcgcgctcgctcgctcactgaggccgcccgggctttgcccgggcggcctcagtgagcgagcgagcgcgcag

SEQ ID NO: 18 - DNA, 48 bp, Artificial Sequence
  Description of Artificial Sequence: Synthetic oligonucleotide
cttgttctttttgcagaagctcagaataaacgctcaactttggcagat

SEQ ID NO: 19 - protein, 7 aa, Artificial Sequence
  Description of Artificial Sequence: Synthetic peptide
DYKDDDK

SEQ ID NO: 20 - protein, 22 aa, Artificial Sequence
  Description of Artificial Sequence: Synthetic peptide
DYKDHDGDYKDHDIDYKDDDDK

SEQ ID NO: 21 - protein, 7 aa, Simian virus 40
PKKKRKV

SEQ ID NO: 22 - protein, 9 aa, Unknown
  Description of Unknown: C-myc NLS sequence
PAAKRVKLD

SEQ ID NO: 23 - protein, 10 aa, Hepatitis delta virus
EGAPPAKRAR

SEQ ID NO: 24 - protein, 8 aa, Unknown
  Description of Unknown: Polyoma T protein NLS sequence
VSRKRPRP

SEQ ID NO: 25 - protein, 18 aa, Unknown
  Description of Unknown: Nucleoplasmin carboxy tail NLS sequence
KRPAATKKAGQAKKKKLD

SEQ ID NO: 26 - protein, 38 aa, Unknown
  Description of Unknown: NLS sequence
NQSSNFGPMKGGNFGGRSSGPYGGGGQYFAKPRNQGGY

SEQ ID NO: 27 - protein, 17 aa, Human T-cell leukemia virus type 1
PKTRRRPRRSQRKRPPT

SEQ ID NO: 28 - DNA, 432 bp, Artificial Sequence
  Description of Artificial Sequence: Synthetic polynucleotide
gccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcgg

SEQ ID NO: 29 - DNA, 516 bp, Artificial Sequence
  Description of Artificial Sequence: Synthetic polynucleotide
gccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcgg

SEQ ID NO: 30 - DNA, 3195 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 31 - DNA, 3273 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 32 - DNA, 321 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 33 - DNA, 393 bp, Artificial Sequence
  Description of Artificial Sequence: Synthetic polynucleotide
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 34 - DNA, 66 bp, Artificial Sequence
  Description of Artificial Sequence: Synthetic oligonucleotide
gactacaaagaccatgacggtgattataaagatcatgacatcgattacaaggatgacgatgacaag

SEQ ID NO: 35 - skipped

SEQ ID NO: 36 - DNA, 21 bp, Artificial Sequence
  Description of Artificial Sequence: Synthetic oligonucleotide
cccaagaagaagaggaaggtc

SEQ ID NO: 37 - DNA, 592 bp, Artificial Sequence
  Description of Artificial Sequence: Synthetic polynucleotide
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 38 - DNA, 600 bp, Artificial Sequence
  Description of Artificial Sequence: Synthetic polynucleotide
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataa

SEQ ID NO: 39 - DNA, 594 bp, Artificial Sequence
  Description of Artificial Sequence: Synthetic polynucleotide
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataa

SEQ ID NO: 40 - DNA, 22 bp, Artificial Sequence
  Description of Artificial Sequence: Synthetic primer
ctctttagctcggcttattcca

SEQ ID NO: 41 - DNA, 22 bp, Artificial Sequence
  Description of Artificial Sequence: Synthetic primer
cgatgatgagcaggacgttaag

SEQ ID NO: 42 - DNA, 24 bp, Artificial Sequence
  Description of Artificial Sequence: Synthetic probe
tcgagatgcacttagcgaaaccca

SEQ ID NO: 43 - DNA, 20 bp, Artificial Sequence
  Description of Artificial Sequence: Synthetic primer
ttcgtcgagatgcacacaag

SEQ ID NO: 44 - DNA, 22 bp, Artificial Sequence
  Description of Artificial Sequence: Synthetic primer
tgctgaagatactgagcaaagg

SEQ ID NO: 45 - DNA, 29 bp, Artificial Sequence
  Description of Artificial Sequence: Synthetic probe
aggttgctcatcggtttaaagatttggga

SEQ ID NO: 46 - DNA, 50 bp, Xenopus sp.
tgcttgttctttttgcagaagctcagaataaacgctcaactttggcagat

SEQ ID NO: 47 - DNA, 225 bp, Artificial Sequence
  Description of Artificial Sequence: Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 48 - protein, 9 aa, Unknown
  Description of Unknown: “LAGLIDADG” family motif peptide
LAGLIDADG